(19) United States
(12) Patent Application Publication
(10) Pub. No.: US 2024/0360247 A1
(43) Pub. Date: Oct. 31, 2024

(54) COMPOSITIONS AND METHODS FOR TREATMENT OF CANCER

(71) Applicant: Johns Hopkins University, Baltimore, MD (US)

(72) Inventor: Subroto Chatterjee, Columbia, MD (US)

(21) Appl. No.: 18/438,286

(22) Filed: Feb. 9, 2024

Related U.S. Application Data
(63) Continuation of application No. PCT/US22/74785, filed on Aug. 10, 2022.
(60) Provisional application No. 63/231,694, filed on Aug. 10, 2021.

(51) Int. Cl. C07K 16/40; A61K 39/00; A61K 45/06; A61P 35/00; G01N 33/573

(52) U.S. Cl. CPC C07K 16/40; A61K 45/06; A61P 35/00; G01N 33/573; A61K 2039/505; C07K 2317/24; C07K 2317/30; G01N 2800/52

(57) ABSTRACT

Compositions in the prevention and treatment of cancers, such as colorectal cancer or diseases associated with abnormal levels of β-1,4-galactosyltransferase-V (β-1,4-GalT-V), include at least one inhibitor of glycosphingolipid synthesis.

The present application is a continuation of International Application No. PCT/US22/074785 filed Aug. 10, 2022 which claims the benefit of U.S. provisional application 63/231,694 filed Aug. 10, 2021, both of which applications are incorporated herein by reference in its entirety.

### STATEMENT REGARDING FEDERALLY SPONSORED RESEARCH

This invention was made with government support under grant number HL107153 awarded by the National Institutes of Health. The government has certain rights in this invention.

### FIELD OF THE INVENTION

Embodiments are directed to compositions that inhibit glycosphingolipid synthesis and their use in the treatment of cancers, such as colorectal cancer.

### SEQUENCE LISTING

The instant application contains a Sequence Listing which has been submitted electronically in XML file format and is hereby incorporated by reference in its entirety. Said XML copy, created on May 20, 2024, is named 348358_11601_SL.xml and is 10,611 bytes in size.

### BACKGROUND

Colorectal cancer (CRC) affects more than 1.4 million people, causes over 690,000 deaths world-wide (P. Favoriti, et al., Worldwide burden of colorectal cancer: a review, Updates Surg. 68(1) (2016) 7-11. doi.org/10.1007/s13304-016-0359-y. H. Brenner, et al., Colorectal cancer, Lancet. 383(9927) (2014) 1490-1502. doi.org/10.1016/S0140-6736(13)61649-9. Ferlay, I. et al., Cancer incidence and mortality worldwide: sources, methods and major patterns in GLOBOCAN 2012, Int. J Cancer. 136(5) (2015) E359-E386. doi.org/10.1002/ijc.29210. M. Arnold, et al., Global patterns and trends in colorectal cancer incidence and mortality, Gut. 66(4) (2017) 683-691. doi.org/10.1136/gutjnl-2015-310912), and is third in prevalence of all cancer types (H. Brenner, C. Stock, M. Hoffmeister, Colorectal cancer screening: the time to act is now, BMC Med. 13 (2015) 262. doi.org/10.1186/s12916-015-0498-x). Current CRC early detection methods are challenged by limited availability, poor patient compliance, and poor test specificity (T. Tanaka, et al., Biomarkers for colorectal cancer, Int. J. Mol. Sci. 11(9) (2010) 3209-3225. doi.org/10.3390/ijms11093209. S. Hundt, U. Haug, H. Brenner, Blood markers for early detection of colorectal cancer: a systematic review, Cancer Epidemiol. Biomarkers Prev. 16(10) (2007) 1935-1953. doi.org/10.1158/1055-9965.EPI-06-0994. K. Simon, V. Balchen, Colorectal cancer development and advances in screening. Clin. Interv. Aging. 11 (2016) 967-976. doi.org/10.2147/CIA.S109285. T. F. Imperiale, et al., Multitarget stool DNA testing for

colorectal-cancer screening, N. Engl. J. Med. 370 (2014) 1287-1297. doi.org/10.1056/NEJMoa1311194).

### SUMMARY

Embodiments of the invention are directed to compositions comprising inhibitors of glycosphingolipid synthesis and methods of use.

In a first aspect, a humanized antibody is provided that can that specifically bind to a β-1,4-galactosyltransferase-V (β-1,4-GalT-V) epitope.

The present humanized antibodies are particularly useful for treating against cancer, particularly cancers that overexpress GalT-V, such as colorectal cancer, renal cancer, and neuroblastomas.

In a second aspect, a method of treating cancer, comprises administering to a subject in need thereof a composition comprising a therapeutically effective amount of: an antibody, wherein the antibody specifically binds to a β-1,4-galactosyltransferase-V (β-1,4-GalT-V) epitope, the antibody comprising: (i) a heavy chain variable region sequence having at least an 80% amino acid sequence identity to: EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 3), and/or, (ii) a light chain variable sequence having at least a 80% amino acid sequence identity to: DVVMTQTPPTLSVTIGQPASISCKSSQSLLDSDGKTYLNWLLQRPGQSPKRLIYLVSK LGSGVPDRFTGSGSGTDFTLKISRVEAEDLGVYYCWQGTHFPRTFGGGTKLEIKR (SEQ ID NO: 4). Preferably, a therapeutically effective amount of at least one inhibitor of glycosphingolipid synthesis is also administered to the subject.

In a third aspect, a method of treating cancer, comprises administering to a subject in need thereof a composition comprising a therapeutically effective amount of: an antibody, wherein the antibody specifically binds to a β-1,4-galactosyltransferase-V (β-1,4-GalT-V) epitope, the antibody comprising: (i) a heavy chain variable region sequence having at least an 83, 84, 85, 86 or 87% amino acid sequence identity to: EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 3), and/or, (ii) a light chain variable sequence having at least a 83, 84, 85, 86 or 87% amino acid sequence identity to: DVVMTQTPPTLSVTIGQPASISCKSSQSLLDSDGKTYLNWLLQRPGQSPKRLIYLVSK LGSGVPDRFTGSGSGTDFTLKISRVEAEDLGVYYCWQGTHFPRTFGGGTKLEIKR (SEQ ID NO: 4). Preferably, a therapeutically effective amount of at least one inhibitor of glycosphingolipid synthesis is also administered to the subject.

In a fourth aspect, a method of treating cancer, comprises administering to a subject in need thereof a composition comprising a therapeutically effective amount of: (a) an antibody, wherein the antibody specifically binds to a β-1,4-galactosyltransferase-V (β-1,4-GalT-V) epitope, the antibody comprising: (i) a heavy chain variable region sequence having at least a 90% or 95% amino acid sequence identity to: EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 3), and/or, (ii) a light chain variable sequence having at least a 90% or 95% amino acid sequence identity to: DVVMTQTPPTLSVTIGQPASISCKSSQSLLDSDGKTYLNWLLQRPGQSPKRLIYLVSK LGSGVPDRFTGSGSGTDFTLKISRVEAEDLGVYYCWQGTHFPRTFGGGTKLEIKR (SEQ ID NO: 4). Preferably, a therapeutically effective amount of at least one inhibitor of glycosphingolipid synthesis is also administered to the subject.

In certain embodiments, the antibody comprises a heavy chain variable region sequence having an amino acid sequence set forth in SEQ ID NO: 3. In certain embodiments, the antibody comprises a light chain variable region sequence having an amino acid sequence set forth in SEQ ID NO: 4. In certain embodiments, the pharmaceutical composition further comprises one or more secondary therapeutic agents. In certain embodiments, the one or more secondary therapeutic agents comprise: chemotherapeutic agents, anti-inflammatory agents, cholesterol lowering agents, insulin, antibodies, peptides, enzymes, adjuvants or combinations thereof. In certain embodiments, the pharmaceutical composition further comprises conjugating the antibody to a detectable agent, a radiotherapeutic agent, a toxin, a radioactive agent, a dye, a peptide, a polynucleotide or a nanoliposome. In certain embodiments, the nanoliposome comprises a therapeutic agent(s). In certain embodiments, the pharmaceutical composition further comprises a peptide having at least a 90% sequence identity to IGAQVYEQVLRSAYAKRNSSVND (SEQ ID NO: 5).

In a fifth aspect, a pharmaceutical composition comprises a therapeutically effective amount of: (i) an antibody comprising (a) a heavy chain variable region sequence nucleic acid sequence having at least an 80%, 85%, 90% or 95% sequence identity to SEQ ID NO: 3, and (b) a light chain variable region sequence nucleic acid sequence having at least a 90% sequence identity to SEQ ID NO: 2; and/or, (ii) a synthetic peptide comprising an amino acid sequence having at least an 80%, 85%, 90% or 95% amino acid sequence to SEQ ID NO: 5.

In certain embodiments, the pharmaceutical composition may also comprise one or more adjuvants and/or pone or more pharmaceutically acceptable carriers. In certain embodiments, the antibody comprises (a) a heavy chain variable region nucleic acid sequence comprising SEQ ID NO: 3, and/or (b) a light chain variable region nucleic acid sequence comprising SEQ ID NO: 2; and/or, the synthetic peptide amino acid sequence comprising SEQ ID NO: 5.

In a sixth aspect, a pharmaceutical composition comprises a therapeutically effective amount of: (a) an antibody, wherein the antibody specifically binds to a β-1,4-galactosyltransferase-V (β-1,4-GalT-V) epitope, the antibody comprising: (i) a heavy chain variable region sequence having at least an 80%, 85%, 90% or 95% amino acid sequence identity to: EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 3), and/or (ii) a light chain variable sequence having at least an 80%, 85%, 90% or 95% amino acid sequence identity to: DVVMTQTPPTLSVTIGQPASISCKSSQSLLDSDGKTYLNWLLQRPGQSPKRLIYLVSK LGSGVPDRFTGSGSGTDFTLKISRVEAEDLGVYYCWQGTHFPRTFGGGTKLEIKR (SEQ ID NO: 4). In certain preferred aspects, the pharmaceutical composition also may comprise a therapeutically effective amount of at least one inhibitor of glycosphingolipid synthesis. In certain embodiments, the antibody comprises a heavy chain variable region sequence having an amino acid sequence set forth in SEQ ID NO: 3. In certain embodiments, the antibody comprises a light chain variable region sequence having an amino acid sequence set forth in SEQ ID NO: 4. In certain embodiments, the at least one inhibitor of glycosphingolipid synthesis comprises: D-threo-1-phenyl-2-d ecanoylamino-3-morpholino-1-propanol (D-PDMP), (1R,2R)-nonanoic acid(2-(2′,3-dihydro-benzo (1, 4) dioxin-6′-yl)-2-hydroxy-1-pyrrolidin-1-ylmethyl-ethyl)-amide-L-tartaric acid salt (Genz-123346), an imide sugar, 1-phenyl-2-decanoylamino-3-morpholino-1-propanol (DMP), 1-phenyl-2-palmitoyl-amino-3-morpholino-1-propanol (PPMP), lipids, ceramides or combinations thereof are unencapsulated or encapsulated by a biodegradable polymer. In certain embodiments, the inhibitor of glycosphingolipid synthesis is D-threo-1-phenyl-2-decanoylamino-3-morpholino-1-propanol (D-PDMP), unencapsulated or encapsulated in a biodegradable polymer (BPD). In certain embodiments, the biodegradable polymer consists of polyethylene glycol and sebacic acid. In certain embodiments, poly(amidoamine) dendrimers based nanoplatforms coupled to an antibody disclosed herein, D-PDMP peptide be useful in cancer detection as well as targeted therapy.

In a seventh aspect, the antibody which specifically binds to a β-1,4-galactosyltransferase-V (β-1,4-GalT-V) epitope is humanized. In certain embodiments, the antibody comprises: (i) a heavy chain variable region sequence having at least an 80%, 85%, 90% or 95% amino acid sequence identity to: EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 3), and/or, (ii) a light chain variable sequence having at least an 80%, 85%, 90% or 95% amino acid sequence identity to:

(SEQ ID NO: 4)
DVVMTQTPPTLSVTIGQPASISCKSSQSLLDSDGKTYLNWLLQRPGQSP
KRLIYLVSKLGSGVPDRFTGSGSGTDFTLKISRVEAEDLGVYYCWQGTH
FPRTFGGGTKLEIKR.

In an eighth aspect, a pharmaceutical composition comprises a therapeutically effective amount of a synthetic peptide comprising an amino acid sequence having at least a 90% amino acid sequence to SEQ ID NO: 5 and preferably at least one adjuvant or at leastg one other pharmaceutically acceptable carrier. In certain embodiments, the synthetic peptide comprises SEQ ID NO: 5.

In an ninth aspect, an expression vector comprises a heavy chain variable region sequence nucleic acid sequence having at least an 80%, 85%, 90% or 95% sequence identity to gaagttcagctggagcagtctggggctgaactggctagacctggggcttcagtgaagttgtcctgtaggacttctggctacacctttaca aactactggatgcagtggattaaacagaggcctggacagggtctggaatggattggggctatgcatcctggacgtgcgtatattaggta caaccagaagttccagggcaaggccacattgactgcagataaatcctccagcacagcttacatgcaactcaacagcttggcatctgag gactctgcggtctattactgtgcaagatggagtgactacgactactggggtcaaggcaccactctcacagtctcctca (SEQ ID NO: 1). In certain embodiments, the vector comprises nucleic acid sequence set forth in SEQ ID NO: 1.

In a tenth aspect, an expression vector comprises a light chain variable region sequence nucleic acid sequence having at least an 80%, 85%, 90% or 95% sequence identity to gatgttgtgatgacccagactccactcactttgtcggttaccattggacaaccagcctccatctcttgcaagtcaagtcagagcctcttag atagtgatggaaagacatatttgaattggttgttacagaggccaggccagtctccaaagcgcctaatctatctggtgtctaaactgggctc tggagtccctgacaggttcactggcagtggatcagggacagatttcacactgaaaatcagcagagtggaggctgaggatttgggagtt tattattgctggcaaggtacacattttcctcggacgttcggtggaggcaccaagctggaaatcaaacgg (SEQ ID NO: 2). In certain embodiments, the vector comprises nucleic acid sequence set forth in SEQ ID NO: 2.

In an eleventh aspect, an expression vector comprises (i) a heavy chain variable region sequence nucleic acid sequence having at least an 80%, 85%, 90% or 95% sequence identity to SEQ ID NO: 3, and/or (ii) a light chain variable region sequence nucleic acid sequence having at least an 80%, 85%, 90% or 95% sequence identity to SEQ ID NO: 2.

In a twelfth aspect, an expression vector comprises (i) a heavy chain variable region sequence nucleic acid sequence comprising SEQ ID NO: 3, and (ii) a light chain variable region sequence nucleic acid sequence comprising SEQ ID NO: 2.

In a thirteenth aspect, a synthetic peptide comprises an amino acid sequence having at least an 80%, 85%, 90% or 95% amino acid sequence to SEQ ID NO: 5. In certain embodiments, the synthetic peptide comprises SEQ ID NO: 5.

In a fourteenth aspect, a method of generating an immune response to β-1,4-galactosyltransferase-V (β-1,4-GalT-V) in a subject in need thereof, comprises administering a therapeutically effective amount of a synthetic peptide comprising an amino acid sequence having at least an 80%, 85%, 90% or 95% amino acid sequence to SEQ ID NO: 5; and preferably an adjuvant ore pharmaceutically acceptable carrier.

In a fifteenth aspect, a method of treating colorectal cancer, comprises administering to a subject a pharmaceutical composition comprising an antibody comprising (a) a heavy chain variable region sequence nucleic acid sequence having at least an 80%, 85%, 90% or 95% sequence identity to SEQ ID NO: 3, and/or (b) a light chain variable region sequence nucleic acid sequence having at least an 80%, 85%, 90% or 95% sequence identity to SEQ ID NO: 2. In certain embodiments, the antibody comprises (a) a heavy chain variable region nucleic acid sequence comprising SEQ ID NO: 3, and (b) a light chain variable region nucleic acid sequence comprising SEQ ID NO: 2. In certain embodiments, the method further comprises administering a therapeutically effective amount of a synthetic peptide comprising an amino acid sequence having at least an 80%, 85%, 90% or 95% amino acid sequence identity to SEQ ID NO: 5 and preferably at least one adjuvant or pharmaceutically acceptable carrier. In certain embodiments, the synthetic peptide comprises SEQ ID NO: 5. In certain embodiments, the method further comprises administering an anti-cancer agent, such as, a chemotherapeutic agent, radiotherapy, a toxin or combinations thereof. In certain embodiments, the anti-cancer agent is a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof. In certain embodiments, the anti-cancer agent is a chemotherapeutic or growth inhibitory agent. For example, a chemotherapeutic or growth inhibitory agent may include an alkylating agent, an anthracycline, an anti-hormonal agent, an aromatase inhibitor, an anti-androgen, a protein kinase inhibitor, a lipid kinase inhibitor, Lyn kinase inhibitor, Src kinase inhibitor, VEGF-R1 R2 inhibitor, EGF-R inhibitor GSK-alpha kinase inhibitor an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof.

In certain embodiments, the anti-cancer agent is an adjuvant. Any substance that enhances an anti-cancer immune response, such as against a cancer-related antigen, or aids in the presentation of a cancer antigen to a component of the immune system may be considered an anti-cancer adjuvant of the present disclosure. In certain embodiments, the method further comprises administering at least one inhibitor of glycosphingolipid synthesis comprising: D-threo-1-phenyl-2-decanoylamino-3-morpholino-1-propanol (D-PDMP), (1R,2R)-nonanoic acid(2-(2′,3′-dihydro-benzo (1, 4) dioxin-6′-yl)-2-hydroxy-1-pyrrolidin-1-ylmethyl-ethyl)-amide-L-tartaric acid salt (Genz-123346), an imide sugar, 1-phenyl-2-decanoylamino-3-morpholino-1-propanol (DMP), 1-phenyl-2-palmitoyl-amino-3-morpholino-1-propanol (PPMP), lipids, ceramides or combinations thereof are unencapsulated or encapsulated by a biodegradable polymer. In certain embodiments, the inhibitor of glycosphingolipid synthesis is D-threo-1-phenyl-2-decanoylamino-3-morpholino-1-propanol (D-PDMP), including D-PDMP that may be admixed with a biodegradable polymer e.g. unencapsulated or encapsulated in a biodegradable polymer (BPD). In certain embodiments, the biodegradable polymer consists of polyethylene glycol and sebacic acid.

In a sixteenth aspect, a method of treating diabetes, atherosclerosis, obesity, autoimmune diseases, or diseases associated with abnormal levels of β-1,4-galactosyltransferase-V (β-1,4-GalT-V), for example systemic lupus erythematosus (SLE), renal cancer, lung cancer, melanoma, neuroblastoma, gliobalstoma, lung, cancer, liver cancer comprises administering to a subject in need thereof, the pharmaceutical compositions embodied herein; the expression vectors embodied herein; the synthetic peptide embodied herein; or combinations thereof.

In a seventeenth aspect, a method of diagnosing and treating colorectal cancer, comprises measuring levels of β-1,4-galactosyltransferase-V (β-1,4-GalT-V) and/or glycosphingolipids in a subject's biological sample, wherein increase β-1,4-GalT-V and/or GSL levels as compared to a healthy subject are elevated, wherein elevated β-1,4-GalT-V and/or GSL levels are diagnostic of colorectal cancer; administering to the subject diagnosed with colorectal cancer, a pharmaceutical composition embodied herein; the expression vectors embodied herein; the synthetic peptide embodied herein; or combinations thereof, thereby, treating colorectal cancer. In certain embodiments, the method further comprises measuring levels of colorectal cancer tumor markers in combination with β-1,4-GalT-V and/or GSL levels. In certain embodiments, colorectal cancer tumor markers comprise: NMT-1, APC, p53, NOTCH-1, β-CATENIN and combinations thereof.

In an eighteenth aspect, a method of monitoring tumor progression (including rectal or colorectal tumors or cancer) using a tagged GalT-V antibody including a fluorescent tagged GalT-V antibody or radioactive isotope (e.g. [¹²⁵I], [⁸⁹]Zr), and other gamma emitting isotopes, or a CF-750-tagged GATT-V antibody.

In a nineteenth aspect, a composition comprises a therapeutically effective amount of: an antibody, wherein the antibody specifically binds to a β-1,4-galactosyltransferase-V (β-1,4-GalT-V) epitope, the antibody comprising: (i) a heavy chain variable region sequence having up or at least an 80%, 82%, 84%, 85%, 86%, 87%, 88%, 90%, 92%, 94%, 96%, 98% or 99% amino acid sequence identity to: EVQLEQSGAELARPGASVKLSCRTSGYTFTNYWMQWIKQ RPGQGLEWIGAMHPGRAYIRYNQKFQGKATLTADKSSSTAYMQLNSLASEDSAVYY CARWSDYDYWGQGTTLTVSS (SEQ ID NO: 7), and/or, (ii) a light chain variable sequence having up to or at least an 80%, 82%, 84%, 85%, 86%, 87%, 88%, 90%, 92%, 94%, 96%, 98% or 99% amino acid sequence identity to: DVVMTQTPLTLSVTIGQPASIS CKSSQSLLDSDGKTYLNWLLQRPGQSPKRLIYLVSKLGSGVPDRFTGSGSGTDFTLKI SRVEAEDLGVYYCWQGTHFPRTFGGGTKLEIKR (SEQ ID NO: 9).

In certain embodiments, a composition comprises a therapeutically effective amount of: an antibody, wherein the antibody specifically binds to a β-1,4-galactosyltransferase-V (β-1,4-GalT-V) epitope, the antibody comprising: (i) a heavy chain variable region amino acid sequence comprising:

(SEQ ID NO: 7)
EVQLEQSGAELARPGASVKLSCRTSGYTFTNYWMQWIKQRPGQGLEWIGAMHPGR
AYIRYNQKFQGKATLTADKSSSTAYMQLNSLASEDSAVYYCARWSDYDYWGQGTT
LTVSS,
and/or,
(ii) a light chain variable amino acid sequence comprising:
(SEQ ID NO: 9)
DVVMTQTPLTLSVTIGQPASISCKSSQSLLDSDGKTYLNWLLQRPGQSPKRLIYLVSK
LGSGVPDRFTGSGSGTDFTLKISRVEAEDLGVYYCWQGTHFPRTFGGGTKLEIKR.

In certain embodiments, an antibody which specifically binds to a β-1,4-galactosyltransferase-V (β-1,4-GalT-V) epitope, comprises a heavy chain variable region amino acid sequence having an amino acid sequence set forth in SEQ ID NO: 7. In certain embodiments, an antibody which specifically binds to a β-1,4-galactosyltransferase-V (β-1,4-GalT-V) epitope comprises a light chain variable region sequence having an amino acid sequence set forth in SEQ ID NO: 9.

In another aspect, a nucleic acid sequence encoding a heavy chain variable region comprises a nucleic acid sequence having at least an 80%, 85%, 90% or 95% sequence identity to:

(SEQ ID NO: 6)
gaagttcagctggagcagtctggggctgaactggctagacctggggctt
cagtgaagttgtcctgtaggacttctggctacacctttacaaactactg
gatgcagtggattaaacagaggcctggacagggtctggaatggattggg
gctatgcatcctggacgtgcgtatattaggtacaaccagaagttccagg
gcaaggccacattgactgcagataaatcctccagcacagcttacatgca
actcaacagcttggcatctgaggactctgcggtctattactgtgcaaga
tggagtgactacgactactggggtcaaggcaccactctcacagtctcct
ca.

In certain embodiments, a nucleic acid sequence encoding a heavy chain variable region comprises SEQ ID NO: 6.

In another aspect, a nucleic acid sequence encoding a light chain variable region comprises a nucleic acid sequence having at least an 80%, 85%, 90%, 92%, 94%, 95%, 96%, 98%, 99% or 100% sequence identity to:

(SEQ ID NO: 8)
gatgttgtgatgacccagactccactcactttgtcggttaccattggac
aaccagcctccatctcttgcaagtcaagtcagagcctcttagatagtga
tggaaagacatatttgaattggttgttacagaggccaggccagtctcca
aagcgcctaatctatctggtgtctaaactgggctctggagtccctgaca
ggttcactggcagtggatcagggacagatttcacactgaaaatcagcag
agtggaggctgaggatttgggagtttattattgctggcaaggtacacat
tttcctcggacgttcggtggaggcaccaagctggaaatcaaacgg.

In certain embodiments, a nucleic acid sequence encoding a heavy chain variable region comprises SEQ ID NO: 8.

In another aspect, an expression vector comprises a nucleic acid sequence having at least an 80%, 85%, 90%, 92%, 94%, 95%, 96%, 98%, 99% or 100% sequence identity to:

(SEQ ID NO: 6)
gaagttcagctggagcagtctggggctgaactggctagacctggggctt
cagtgaagttgtcctgtaggacttctggctacacctttacaaactactg
gatgcagtggattaaacagaggcctggacagggtctggaatggattggg
gctatgcatcctggacgtgcgtatattaggtacaaccagaagttccagg
gcaaggccacattgactgcagataaatcctccagcacagcttacatgca
actcaacagcttggcatctgaggactctgcggtctattactgtgcaaga
tggagtgactacgactactggggtcaaggcaccactctcacagtctcct
ca.

In another aspect, an expression vector comprises a nucleic acid sequence having at least an 80%, 85%, 90%, 92%, 94%, 95%, 96%, 98%, 99% or 100% sequence identity to:

(SEQ ID NO: 8)
gatgttgtgatgacccagactccactcactttgtcggttaccattggac
aaccagcctccatctcttgcaagtcaagtcagagcctcttagatagtga
tggaaagacatatttgaattggttgttacagaggccaggccagtctcca
aagcgcctaatctatctggtgtctaaactgggctctggagtccctgaca
ggttcactggcagtggatcagggacagatttcacactgaaaatcagcag
agtggaggctgaggatttgggagtttattattgctggcaaggtacacat
tttcctcggacgttcggtggaggcaccaagctggaaatcaaacgg.

In another aspect, an expression vector encodes:
(i) a heavy chain variable region comprising a nucleic acid sequence having at least an 80%, 85%, 90%, 92%, 94%, 95%, 96%, 98%, 99% or 100% sequence identity to:

(SEQ ID NO: 6)
gaagttcagctggagcagtctggggctgaactggctagacctggggctt
cagtgaagttgtcctgtaggacttctggctacacctttacaaactactg
gatgcagtggattaaacagaggcctggacagggtctggaatggattggg
gctatgcatcctggacgtgcgtatattaggtacaaccagaagttccagg
gcaaggccacattgactgcagataaatcctccagcacagcttacatgca
actcaacagcttggcatctgaggactctgcggtctattactgtgcaaga
tggagtgactacgactactggggtcaaggcaccactctcacagtctcct
ca.

and,
(ii) a light chain variable region comprising a nucleic acid sequence having at least an 80%, 85%, 90%, 92%, 94%, 95%, 96%, 98%, 99% or 100% sequence identity to:

(SEQ ID NO: 8)
gatgttgtgatgacccagactccactcactttgtcggttaccattggac
aaccagcctccatctcttgcaagtcaagtcagagcctcttagatagtga
tggaaagacatatttgaattggttgttacagaggccaggccagtctcca
aagcgcctaatctatctggtgtctaaactgggctctggagtccctgaca
ggttcactggcagtggatcagggacagatttcacactgaaaatcagcag
agtggaggctgaggatttgggagtttattattgctggcaaggtacacat
tttcctcggacgttcggtggaggcaccaagctggaaatcaaacgg.

In another aspect, a method of treating colorectal cancer, comprises administering to a subject a pharmaceutical composition comprising an antibody comprising (a) a heavy chain variable region sequence nucleic acid sequence having at least an 80%, 85%, 90%, 92%, 94%, 95%, 96%, 98%, 99% or 100% sequence identity to SEQ ID NO: 6, and/or (b) a light chain variable region sequence nucleic acid sequence having at least an 80%, 85%, 90%, 92%, 94%, 95%, 96%, 98%, 99% or 100% sequence identity to SEQ ID NO: 8. In certain embodiments, the antibody comprises (a) a heavy chain variable region nucleic acid sequence comprising SEQ ID NO: 6, and (b) a light chain variable region nucleic acid sequence comprising SEQ ID NO: 8. In certain aspects, the subject is identified as suffering from colorectal cancer, and the pharmaceutical composition is administered to the identified subject. In certain embodiments, the method further comprises administering an anti-cancer agent, such as, a chemotherapeutic agent, radiotherapy, a toxin or combinations thereof. In certain embodiments, the anti-cancer agent is a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof. In certain embodiments, the anti-cancer agent is a chemotherapeutic or growth inhibitory agent. For example, a chemotherapeutic or growth inhibitory agent may include an alkylating agent, an anthracycline, an anti-hormonal agent, an aromatase inhibitor, an anti-androgen, a protein kinase inhibitor, a lipid kinase inhibitor, Lyn kinase inhibitor, Src kinase inhibitor, VEGF-RT R2 inhibitor, EGF-R inhibitor GSK-alpha kinase inhibitor an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof.

In treatment of colorectal cancer in a subject, treatment may comprise administering to the subject diagnosed with colorectal cancer, a pharmaceutical composition disclosed herein; the expression vectors embodied herein; the synthetic peptide embodied herein; or combinations thereof, measuring levels of β-1,4-galactosyltransferase-V (β-1,4-GalT-V) and/or glycosphingolipids in a subject's biological sample, wherein a decrease in β-1,4-GalT-V and/or GSL levels (e.g. using fluorescent tagged glycosphingolipid antibody) as compared to a baseline, are indicative of a decrease in colorectal cancer cells and treatment of the colorectal cancer. In certain embodiments, the dose of the compositions administered to the subject are modulated based on the progression of the colorectal cancer.

In certain embodiments, the cancer being treated or monitored is Dukes B (stage II) or Dukes C (stage III) colorectal cancer.

In yet further aspects, methods are provided for treating a patient suffering from or susceptible to macular degeneration, comprising administering to the subject an effective amount of one or more pharmaceutical composition, peptide and/or expression vector as disclosed herein, including combinations thereof. In certain aspects, the subject may be identified as suffering from macular degeneration and the one or more pharmaceutical composition, peptide and/or expression vector as disclosed herein is administered to the identified subject. In certain preferred embodiments, the subject is a human.

In additional aspects, methods are provided for treating a patient suffering from or susceptible to Alzheimer's disease, comprising administering to the subject an effective amount of one or more pharmaceutical composition, peptide and/or expression vector as disclosed herein, including combinations thereof. In certain aspects, the subject may be identified as suffering from Alzheimer's disease and the one or more pharmaceutical composition, peptide or expression vector as disclosed herein is administered to the identified subject. In certain preferred embodiments, the subject is a human.

In further aspects, methods are provided for treating a patient suffering from or susceptible to migraines or migraine pain, comprising administering to the subject an effective amount of one or more pharmaceutical composition, peptide and/or expression vector as disclosed herein, including combinations thereof. In certain aspects, the subject may be identified as suffering from migraines or migraine pain and the one or more pharmaceutical composition, peptide and/or expression vector as disclosed herein is administered to the identified subject. In certain preferred embodiments, the subject is a human.

In yet additional aspects, methods are provided for treating a patient suffering from or susceptible to Metabolic syndrome, comprising administering to the subject an effective amount of one or more pharmaceutical composition, peptide and/or expression vector as disclosed herein, including combinations thereof. In certain aspects, the subject may be identified as suffering from Metabolic syndrome and the one or more pharmaceutical composition, peptide and/or expression vector as disclosed herein is administered to the identified subject. In certain preferred embodiments, the subject is a human.

Other aspects are described infra.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

The term “administering,” as used herein, refers to any mode of transferring, delivering, introducing, or transporting a therapeutic agent to a subject in need of treatment with such an agent. Such modes include, but are not limited to, oral, topical, intravenous, intraperitoneal, intramuscular, intradermal, intranasal, and subcutaneous administration. As used herein, the term “agent” is meant to encompass any molecule, chemical entity, composition, drug, therapeutic agent, chemotherapeutic agent, or biological agent capable of modulating β1,4-Galactosyltransferase V (BGA) expression or activity. The term includes small molecule compounds, antisense oligonucleotides, siRNA reagents, antibodies, antibody fragments bearing epitope recognition sites, such as Fab, Fab′, F(ab′)2 fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the invention at any stage during clinical trials, during pre-trial testing, or following FDA-approval.

As used herein, the term “antibody” is inclusive of all species, including human and humanized antibodies and the antigenic target, can be from any species. Thus, an antibody, for example, which binds to an antigen “X” can be mouse anti-human X, human anti-human X; humanized anti-human X, goat anti-human X; goat anti-mouse X; rat anti-human X; mouse anti-rat X and the like. The combinations of antibody generated in a certain species against an antigen target, e.g. “X”, from another species, or in some instances the same species (for example, in autoimmune or inflammatory response) are limitless and all species are embodied in this invention. The term antibody is used in the broadest sense and includes fully assembled antibodies, monoclonal antibodies (including human, humanized or chimeric antibodies), polyclonal antibodies, multispecific antibodies (e.g., bispecific antibodies), and antibody fragments that can bind antigen (e.g., Fab′, F′(ab)₂, Fv, single chain antibodies, diabodies), comprising complementarity determining regions (CDRs) of the foregoing as long as they exhibit the desired biological activity. Examples of a bispecific antibody include a combination of the GalT-V antibody with another antibody e.g. Lactoslceramide, Lyn kinase, Src kinase, VEGF-RT, R2, EGF-R GSK-alpha kinase.

By “antisense oligonucleotides” or “antisense compound” is meant an RNA or DNA molecule that binds to another RNA or DNA (target RNA, DNA). For example, if it is an RNA oligonucleotide it binds to another RNA target by means of RNA-RNA interactions and alters the activity of the target RNA. An antisense oligonucleotide can upregulate or downregulate expression and/or function of a particular polynucleotide. The definition is meant to include any foreign RNA or DNA molecule which is useful from a therapeutic, diagnostic, or other viewpoint. Such molecules include, for example, antisense RNA or DNA molecules, interference RNA (RNAi), micro RNA, decoy RNA molecules, siRNA, enzymatic RNA, short, hairpin RNA (shRNA), therapeutic editing RNA and agonist and antagonist RNA, antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds may be introduced in the form of single-stranded, double-stranded, partially single-stranded, or circular oligomeric compounds.

As used in this specification and the appended claims, the term “or” is generally employed in its sense including “and/or” unless the content clearly dictates otherwise.

As used herein, the term “chemotherapeutic agent,” consistent with its use in the art, refers to one or more agents known, or having characteristics known to, treat or contribute to the treatment of cancer. In particular, chemotherapeutic agents include pro-apoptotic, cytostatic, and/or cytotoxic agents. In some embodiments, a chemotherapeutic agent can be or include alkylating agents, anthracyclines, cytoskeletal disruptors (e.g., microtubule targeting moieties such as taxanes, maytansine, and analogs thereof, of), epothilones, histone deacetylase inhibitors HDACs), topoisomerase inhibitors (e.g., inhibitors of topoisomerase I and/or topoisomerase II), kinase inhibitors, nucleotide analogs or nucleotide precursor analogs, peptide antibiotics, platinum-based agents, retinoids, vinca alkaloids, and/or analogs that share a relevant anti-proliferative activity. In some embodiments, a chemotherapeutic agent can be or include Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g., DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, or a combination thereof. In some embodiments, a chemotherapeutic agent can be utilized in the context of an antibody-drug conjugate. In some embodiments, a chemotherapeutic agent is one found in an antibody-drug conjugate selected from the group consisting of: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-SME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine.

As used herein, the term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the term “diagnosis” refers to determining whether, and/or the qualitative of quantitative probability that, a subject has or will develop a disease, disorder, condition, or state. For example, in diagnosis of cancer, diagnosis can include a determination regarding the risk, type, stage, malignancy, or other classification of a cancer. In some instances, e.g., as set forth herein, a diagnosis can be or include a determination relating to prognosis and/or likely response to one or more general or particular therapeutic agents or regimens.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A disease or disorder is “alleviated” if the severity of a symptom of the disease or disorder, the frequency with which such a symptom is experienced by a patient, or both, is reduced.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, a dosing regimen is or has been correlated with a desired therapeutic outcome, when administered across a population of patients. As used herein, a “controlled release dosage formulation” refers to a formulation of a drug that offers prolonged release at a specific controllable rate.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. Determination of a therapeutically effective amount, as well as other factors related to effective administration of a compound of the present invention to a subject of this invention, including dosage forms, routes of administration, and frequency of dosing, may depend upon the particulars of the condition that is encountered, including the subject and condition being treated or addressed, the severity of the condition in a particular subject, the particular compound being employed, the particular route of administration being employed, the frequency of dosing, and the particular formulation being employed. Determination of a therapeutically effective treatment regimen for a subject of this invention is within the level of ordinary skill in the medical or veterinarian arts. In clinical use, an effective amount may be the amount that is recommended by the U.S. Food and Drug Administration, or an equivalent foreign agency. The amount of active ingredient that can be combined with the carrier materials to produce a single dosage form varies depending upon the subject being treated and the particular mode of administration.

The term “high affinity” for an antibody refers to an antibody having a K_{D} of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target antigen. However, “high affinity” binding can vary for other antibody isotypes. For example, “high affinity” binding for an IgM isotype refers to an antibody having a K_{D} of 10⁻⁶ M or less, 10⁻⁷ M or less, or 10⁻⁸ M or less.

The term “enhancement,” “enhance,” “enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified activity of at least about 5%, 10%, 25%, 35%, 40%, 50%, 60%, 75%, 80%, 90%, 95%, 97%, 98%, 99% or 100%.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used herein, an “inhibitor” of glycosphingolipid synthesis or of glucosylceramide synthesis inhibits the synthesis of these molecules including those associated in the cycle of the synthesis. The inhibition of synthesis of these molecules can be measured by any standard assay. See, for example, the methods in the examples section which follows.

As used herein, “inhibition” or “decrease” of β1,4-Galactosyltransferase V reduces the amount of β1,4-Galactosyltransferase V in the cell by greater than about 20%, 40%, 60%, 80%, 85%, 90%, 95%, or 100%. The amount of β1,4-Galactosyltransferase V can be determined by well-known methods including, but are not limited to, densitometer, fluorometer, radiography, luminometer, antibody-based methods and activity measurements.

The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or a decrease or reduction in the specified activity of at least about 5%, 10%, 25%, 35%, 40%, 50%, 60%, 75%, 80%, 90%, 95%, 97%, 98%, 99% or 100%. These terms are intended to be relative to a reference or control.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, for example, using a biosensor system such as a BIACORE™ system.

As used herein, “modulate,” “modulates” or “modulation” refers to enhancement (e.g., an increase) or inhibition (e.g., diminished, reduced or suppressed) of a specified activity or level (e.g. amount of mRNA, amount of protein, expression of a marker, amount of GSL etc.). Relative to a control level, the level that is to be determined may be an increased level. As used herein, the term “increased” with respect to a level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level. Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered complete when onset of a disease, disorder, or condition has been delayed for a predefined period of time.

As used herein, the term “prognosis” refers to determining the qualitative of quantitative probability of at least one possible future outcome or event. As used herein, a prognosis can be a determination of the likely course of a disease, disorder, or condition such as cancer in a subject, a determination regarding the life expectancy of a subject, or a determination regarding response to therapy, e.g., to a particular therapy.

As used herein, the term “prognostic information” refers to information useful in providing a prognosis. Prognostic information can include, without limitation, biomarker status information.

The term “sample” as used herein refers to a biological sample obtained for the purpose of evaluation in vitro. In embodiments, the sample may comprise a body fluid. In some embodiments, the body fluid includes, but is not limited to, whole blood, plasma, serum, lymph, breast milk, saliva, mucous, semen, cellular extracts, inflammatory fluids, cerebrospinal fluid, vitreous humor, tears, vitreous, aqueous humor, or urine obtained from the subject. In some aspects, the sample is a composite panel of two or more body fluids. In exemplary aspects, the sample comprises blood or a fraction thereof (e.g., plasma, serum, or a fraction obtained via leukapheresis).

As used herein, the terms “prevent,” “preventing” and “prevention” in the context of the administration of a therapy to a subject refer to the prevention or inhibition of the recurrence, onset, and/or development of a disease or disorder or a symptom thereof in a subject resulting from the administration of a therapy (e.g., a prophylactic agent), or a combination of therapies (e.g., a combination of prophylactic agents).

By “reduces” is meant a negative alteration of at least 10%, 25%, 50%, 75%, or 100% as compared to a reference.

By “reference” is meant a standard or control condition.

As used herein, an antibody that “specifically binds” to a polypeptide or epitope is intended to refer to a an antibody that binds to a polypeptide or epitope with a K_{D} of 1×10⁻⁷ M or less, or 5×10⁻⁸ M or less, or 3×10⁻⁸ M or less, more preferably 1×10⁻⁸ M or less, or 5×10⁻⁹ M or less. Therefore, the terms “specific binding” or “specifically binding” when used in reference to the interaction of a protein and an antibody or alternative protein scaffold or peptoid or aptamers, means that the interaction is dependent upon the presence of a particular structure (i.e., the antigenic determinant or epitope) on the protein; in other words the antibody is recognizing and binding to a specific protein structure rather than to proteins in general. Thus, an antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

As used herein, a “sustained release dosage formulation” is a formulation of a drug designed to release the drug at a predetermined rate in order to maintain a constant drug concentration for a specific period of time with minimum side effects. Optionally, the period of time is 30 minutes or more, e.g., 2-4 hours or more, e.g., 3-8 hours or more, e.g., 4-24 hours or
more, e.g., 1-3 days or more, e.g., 2-7 days or more, e.g., 4-14 days or more, e.g., 7 days or more, e.g., 14 days to a month or more.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1I are a series of immunostains and graphs demonstrating that CRC tissue strongly immunoreacts to an anti-β-1,4-GalT-V antibody and shows increased LCS activity and B4GALT5 expression. (FIG. 1A) Normal colon score 2 (20× magnification). (FIG. 1B) Cytoplasmic staining of endothelium (20×). (FIG. 1C) Colon cancer case score 1 (20×). (FIG. 1D) Colon cancer case score 2 (20×). (FIG. 1E) Colon cancer case score 3 (20×). (FIG. 1F) CRC tissues overexpress β-1,4-GalT-V. Visibly normal and CRC tissues (50 mg each) were homogenized in RIPA buffer and centrifuged at 1000 rpm. The supernatant was used to measure β-1,4-GalT-V mass by ELISA. This assay was then run in triplicate with N=10, for both normal and tumor samples. Averages±SEm values are shown,*P=0.0340. Unpaired t-test was used to determine statistical significance. (FIG. 1G) Increased LCS activity in colorectal tumors. Averages±SEm,**P=0.0052. (FIG. 1H) APC, NMT1, and TP53 genes showed increased expression in tumors, compared to that of normal samples. (FIG. 1I) B4GALT5 specifically showed increased expression, while B4GALT6 and UGCG relatively did not. Averages±SEM, with N÷4 for both patient normal and tumor samples. Ordinary one-way ANOVA was utilized for statistical analysis.

FIGS. 2A-2G are a series of graphs demonstrating that LacCer mass is increased in CRC tissue. Visibly normal and CRC tissue (50 mg each) were homogenized in chloroform-methanol (2:1), in the presence of internal sphingolipid standards. Lipid extracts were then subjected to LC-MS to investigate changes in the levels of sphingolipid species, in CRC or normal tissues, with (FIG. 2A) Cer, (FIG. 2B) DHCer, (FIG. 2C) GalCer and GlcCer, (FIG. 2D) dihydroGalCer/dihydroGlcCer, (FIG. 2E) LacCer, and (FIG. 2F) DHLacCer. Among the GSL investigated, only (FIG. 2E) LacCer levels were statistically and significantly increased in colorectal tumors (*P=0.0112). For Cer and dihydroGalCer/dihydroGlcCer, N=10 (normal) and N=9 (tumor); DHCer, N=9 (normal) and N=10 (tumor); and for GalCer and GlcCer, LacCer, and DHLacCer N=10 (both normal and tumor). (FIG. 2G) Normal and tumor tissues were evaluated via LC-MS for sphingomyelin and DHSM levels. No statically significant differences were found in sphingomyelin values for normal vs. tumor. In contrast, tumor samples showed significantly elevated DHSM values compared to normal (**P=0.0059). For sphingomyelin, N=10 (normal) and N=6 (tumor); and for DHSM, N=10 (both normal and tumor). Averages±SEm values, and unpaired t-tests were used to determine statistical significance.

FIGS. 3A-3H are a series of graphs and fluorescent stains demonstrating that pharmacologic inhibition of GSL synthesis dose-dependently decreases proliferation, and reduces β-1,4-GalT-V protein expression in HCT-116. D-PDMP exerted a dose- and time-dependent decrease at (FIG. 3A) 24 and (FIG. 3B) 96 h in HCT-116 cell proliferation, compared to controls, with the maximal effective dose at 20 μM.*P≤0.05,**P≤0.01,***P≤0.001. No difference was found in UGCG immunofluorescence in (FIG. 3D) D-PDMP-treated cells, compared to that of (FIG. 3C) control at 24 h. However, D-PDMP treatment at (FIG. 3F) 24 and (FIG. 3H) 96 h reduced GalT-V fluorescence compared to that of untreated controls (E and G, respectively).

FIGS. 4A-4H are a series of graphs demonstrating that D-PDMP treatment reduces the levels of several sphingolipids in HCT-116. HCT-116 cells were seeded (105) onto sterilized (100-mm²) plastic Petri dishes in 10 mL of medium for 24 h. Media was then changed to 2% serum-containing media, with and without D-PDMP (10 μM). After 24 h, media was removed, and total lipids were extracted, using hexane-isopropanol (3:2, by volume) in the presence of sphingolipid internal standards, and subject to MS. D-PDMP treatment (designated D10 in the x-axis) reduced levels of (FIG. 4A) Cer, (FIG. 4B) DHCer, (FIG. 4C) monohexosylceramides, (FIG. 4D) dihydroGlc/galceramides, (FIG. 4E) dihexosylceramide, (FIG. 4F) DHLacCer but not that of (FIG. 4G) sphingomyelin nor (FIG. 4H) DHSM, at 10 μM, compared to control values (designated as C in the x-axis). Data represent averages±SEm, N=3 biological replicates for control and 10 μM D-PDMP in (FIGS. 4A-4H), and the unpaired t-tested was used for statistical analysis.*P≤0.05,**P≤0.01]\

FIGS. 5A-5D are a series of immunostains demonstrating that CRC tissue strongly immunoreacts to an anti-β-1,4-GalT-V antibody.

FIG. 6 shows an immunostaining of colon cancer section with GalT-V antibody.

FIG. 7 is a schematic representation showing the sphingolipid synthesis pathway.

FIG. 8 is a graph demonstrating that treatment with GalT-V antibody against GalT-V dose dependently decreases proliferation ion HCT-116 cells. HCT-116 cells were seeded (1×10⁴ cells/well) in a 96-well tray and grown in medium supplemented with 10% fetal calf serum. After 24 hrs, fresh medium containing ³H-thymidine (5 μCi/ml) plus various dilutions of GalT-V monoclonal antibody was added. Treatment with D-PDMP (5 μM) served as a positive control. Following incubation for 24 hrs, the incorporation of radioactivity into DNA was measured by scintillation spectrometry. GalT-V antibody dose-dependently decreased HCT-116 cell proliferation P**≤0.01, P***≤0.001 and P****≤0.0001 (N=5).

FIG. 9 is a graph demonstrating that treatment with GalT-V antibody against GalT-V dose-dependently decreases proliferation in mouse colorectal cancer cells. Mouse colorectal cancer cells were seeded (1×10⁴ cells/well) in a 96 well tray and grown in minimum essential medium supplemented with 10% fetal calf serum and processed as described in FIG. 8 above. It is noted that GalT-V antibody dose-dependently decreased MC-38 cell proliferation (N=5).

FIGS. 10A-10I are a series of photo images and a graph demonstrating that VEGF induced tube formation was mitigated by antibody against β-GalT-V and lactosylceramide antibody. Human umbilical vein endothelial cells were incubated for one hour with various dilutions of β-GalT-V antibody or LacCer antibody, followed by treatment with VEGF for 6 hours. Tube formation assays were then performed. The letters on the treatment axis of the graph (FIG. 10I) represent the treatments shown in FIGS. 10A-10H.

FIGS. 11A-11D are a series of photographs demonstrating that treatment with β-1,4 GalT-V antibody or a biopolymer-encapsulated D-PDMP prevented tumor growth in normal female mice. Normal female mice (C57BL6) 32 weeks of age, were shaven. The dorsal area was cleaned with an alcoholic swab and 100 μL of colorectal cancer cells; HCT-116 (4×10⁶) suspension was injected. On week later, 100 μL of monoclonal antibody against B-1,4 GalT-V was injected daily into the site of the tumor cell injection (FIGS. 11A, 11B) or a β-1,4 GalT-V inhibitor (5 mpk of a biopolymer-encapsulated D-PDMP (FIGS. 11C, 11D) for three weeks. The dorsal area was shaven again with Nair as hair had grown back and mice photographed. Note that no tumor growth was observed in the treated mice (FIGS. 11A-11D).

FIG. 12 is a schematic representation depicting an outline of reactions as to how β-galactosyltransferase (β-GalT-V) may contribute to colorectal cancer and the novel approaches herein to prevent it.

FIG. 13 is a schematic of the GalT-V antibody treatment model of Example 4.

FIG. 14 sets forth results showing treatment with GalT-V antibody did not alter body weight in NOD-SCID mice.

FIG. 15 (includes FIGS. 15A-15B) shows treatment with GalT-V antibody dose-dependently reduced tumor volume in NOD-SCID mice inoculated with HCT-116 cells.

FIG. 16 (includes FIGS. 16A-16C) shows optical imaging of mice bearing HCT-116 rectal orthotopic tumor.

FIG. 17 shows q-RT-PCR analysis of B4GALT-V, CEA, and NMT-1 gene expression in CRC mice.

FIG. 18 (includes FIGS. 18A-18B) show in FIG. 18A ELISA assays that demonstrate that treatment with GalT-V-Ab reduced the mass of GalT-V in plasma, and in FIG. 18B HPTLC and densitometric analysis demonstrating that treatment with GalT-V-Ab reduced the mass of LacCer in tumor tissue compared with placebo.

FIG. 19 shows cell surface localization of GalT-V antibody determined by confocal microscopy (Example 5).

FIG. 20 shows GalT-V antibody internalized (37° C.) as determined by confocal microscopy.

FIG. 21 shows [⁸⁹Zr] GalT-V antibody binding in human coronary arterial endothelial cells (HCAEC) and human colorectal cancer cells.

FIG. 22 shows [⁸⁹Zr] GalT-V antibody binding in human coronary arterial endothelial cells (HCAEC) and human colorectal cancer cells.

FIG. 23 shows D-PDMP inhibits zirconium tagged GalT-V antibody binding in human colorectal cancer cells.

FIG. 24 shows specificity of binding and internalization of [⁸⁹Zr] GalT-V antibody in human colorectal cancer cells.

FIG. 25 and FIG. 26 showtime-dependent binding and internalization of [⁸⁹Zr] GalT-V antibody in human colorectal cancer cells.

FIG. 27 and FIG. 28 show in vivo xenogen fluorescence images of human CRC tumor bearing mice at specified time periods.

FIG. 29A-FIG. 29C shows distribution of CF-750 GalT-V antibody fluorescence in individual tissues from a subcutaneous/xenograft tumor bearing mice.

### DETAILED DESCRIPTION

The invention is based, in part, on the finding that β-galactosyltransferase (β-GalT-V) plays a role in human CRC, and that its inhibition also mitigates tumor cell proliferation. Samples from colorectal cancer subjects were found to be immunoreactive to β-1,4-GalT-V antibodies. In addition, β-1,4-GalT-V mass, mRNA expression, enzymatic activity, and GSL end-product levels were assessed. The effect of a GSL glycosyltransferase inhibitor in human CRC cell lines was examined. These results described in detail in the examples section provide new insights into the pathogenesis of and reveal promising detection/prognostic biomarkers for CRC. Applications include biomarkers useful for screening for cancer, particularly colorectal cancer and precursor tumors to colorectal cancers (e.g., advanced adenomas). Compositions are also described for use in the treatment of cancers, such as colorectal cancer and the like.

Colorectal cancers include, without limitation, colon cancer, rectal cancer, and combinations thereof. Colorectal cancers include metastatic colorectal cancers and non-metastatic colorectal cancers. Colorectal cancers include cancer located in the proximal part of the colon cancer and cancer located the distal part of the colon. Colorectal cancers include colorectal cancers at any of the various possible stages known in the art, including, e.g., Stage I, Stage II, Stage III, and Stage IV colorectal cancers (e.g., stages 0, I, IIA, IIB, IIC, IIIA, IIIB, IIIC, IVA, IVB, and IVC). Colorectal cancers include all stages of the Tumor/Node/Metastasis (TNM) staging system. With respect to colorectal cancer, T can refer to whether the tumor grown into the wall of the colon or rectum, and if so by how many layers; N can refer to whether the tumor has spread to lymph nodes, and if so how many lymph nodes and where they are located; and M can refer to whether the cancer has spread to other parts of the body, and if so which parts and to what extent. Particular stages of T, N, and M are known in the art. T stages can include TX, TO, Tis, T1, T2, T3, T4a, and T4b; N stages can include NX, N0, N1a, N1b, N1c, N2a, and N2b; M stages can include M0, M1a, and M1b. Moreover, grades of colorectal cancer can include GX, G1, G2, G3, and G4. Various means of staging cancer, and colorectal cancer in particular, are well known in the art summarized, e.g., cancer.net/cancer-types/colorectal-cancer/stages.

In certain embodiments, the present disclosure includes screening of early stage colorectal cancer. Early stage colorectal cancers can include, e.g., colorectal cancers localized within a subject, e.g., in that they have not yet spread to lymph nodes of the subject, e.g., lymph nodes near to the cancer (stage NO), and have not spread to distant sites (stage M0). Early stage cancers include colorectal cancers corresponding to, e.g., Stages 0 to II C.

Thus, colorectal cancers include, among other things, pre-malignant colorectal cancer (e.g., advanced adenomas) and malignant colorectal cancer. Methods and compositions of the present disclosure are useful for screening of colorectal cancer in all of its forms and stages, including without limitation those named herein or otherwise known in the art, as well as all subsets thereof. Accordingly, the person of skill in art will appreciate that all references to colorectal cancer provided here include, without limitation, colorectal cancer in all of its forms and stages, including without limitation those named herein or otherwise known in the art, as well as all subsets thereof.

Accordingly, in certain embodiments, pharmaceutical compositions in the prevention and treatment of cancer, e.g. colorectal cancer comprise administration of inhibitors of glycosphingolipid synthesis to subjects in need thereof.

In certain embodiments, a method of treating cancer, comprises administering to a subject in need thereof a composition comprising a therapeutically effective amount of: (a) an antibody, wherein the antibody specifically binds to a β-1,4-galactosyltransferase-V (β-1,4-GalT-V) epitope, the antibody comprising: (i) a heavy chain variable region sequence having at least a 90% amino acid sequence identity to: EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 3), and, (ii) a light chain variable sequence having at least a 90% amino acid sequence identity to: DVVMTQTPPTLSVTIGQPASISCKSSQSLLDSDGKTYLNWLLQRPGQSPKRLIYLVSK LGSGVPDRFTGSGSGTDFTLKISRVEAEDLGVYYCWQGTHFPRTFGGGTKLEIKR (SEQ ID NO: 4), and, (b) a therapeutically effective amount of at least one inhibitor of glycosphingolipid synthesis. In certain embodiments, the antibody comprises a heavy chain variable region sequence having an amino acid sequence set forth in SEQ ID NO: 3. In certain embodiments, the antibody comprises a light chain variable region sequence having an amino acid sequence set forth in SEQ ID NO: 4.

In certain embodiments, a pharmaceutical composition comprises a therapeutically effective amount of: (i) an antibody comprising (a) a heavy chain variable region sequence nucleic acid sequence having at least a 90% sequence identity to SEQ ID NO: 3, and (b) a light chain variable region sequence nucleic acid sequence having at least a 90% sequence identity to SEQ ID NO: 2; and, (ii) a synthetic peptide comprising an amino acid sequence having at least a 90% amino acid sequence to SEQ ID NO: 5; and, (iii) an adjuvant. In certain embodiments, the antibody comprises (a) a heavy chain variable region nucleic acid sequence comprising SEQ ID NO: 3, and (b) a light chain variable region nucleic acid sequence comprising SEQ ID NO: 2; and, the synthetic peptide amino acid sequence comprising SEQ ID NO: 5.

In certain embodiments, a pharmaceutical composition comprises a therapeutically effective amount of: (i) an antibody comprising (a) a heavy chain variable region sequence nucleic acid sequence having at least a 90% sequence identity to SEQ ID NO: 6, and (b) a light chain variable region sequence nucleic acid sequence having at least a 90% sequence identity to SEQ ID NO: 8; and, (ii) a synthetic peptide comprising an amino acid sequence having at least a 90% amino acid sequence to SEQ ID NO: 5; and, (iii) an adjuvant. In certain embodiments, the antibody comprises (a) a heavy chain variable region nucleic acid sequence comprising SEQ ID NO: 6, and (b) a light chain variable region nucleic acid sequence comprising SEQ ID NO: 8; and, the synthetic peptide amino acid sequence comprising SEQ ID NO: 5.

In certain embodiments, a pharmaceutical composition comprises a therapeutically effective amount of: (a) an antibody, wherein the antibody specifically binds to a β-1,4-galactosyltransferase-V (β-1,4-GalT-V) epitope, the antibody comprising: (i) a heavy chain variable region sequence having at least a 90%, 95%, 96%, 97%, 98%, 99% or 100% amino acid sequence identity to EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 7), and (ii) a light chain variable sequence having at least a 90%, 95%, 96%, 97%, 98%, 99% or 100% amino acid sequence identity to: DVVMTQTPLTLSVTIGQPASISCKSSQSLLDSDGKTYLNWLLQRPGQSPKRLIYLVSK LGSGVPDRFTGSGSGTDFTLKISRVEAEDLGVYYCWQGTHFPRTFGGGTKLEIKR (SEQ ID NO: 9) and, (b) a therapeutically effective amount of at least one inhibitor of glycosphingolipid synthesis. In certain embodiments, the antibody comprises a heavy chain variable region sequence having an amino acid sequence set forth in SEQ ID NO: 7. In certain embodiments, the antibody comprises a light chain variable region sequence having an amino acid sequence set forth in SEQ ID NO: 9.

In certain embodiments, a pharmaceutical composition comprises a therapeutically effective amount of: (a) an antibody, wherein the antibody specifically binds to a β-1,4-galactosyltransferase-V (β-1,4-GalT-V) epitope, the antibody comprising: (i) a heavy chain variable region sequence having at least a 90% amino acid sequence identity to: EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 3), and, (ii) a light chain variable sequence having at least a 90% amino acid sequence identity to: DVVMTQTPPTLSVTIGQPASISCKSSQSLLDSDGKTYLNWLLQRPGQSPKRLIYLVSK LGSGVPDRFTGSGSGTDFTLKISRVEAEDLGVYYCWQGTHFPRTFGGGTKLEIKR (SEQ ID NO: 4), and, (b) a therapeutically effective amount of at least one inhibitor of glycosphingolipid synthesis. In certain embodiments, the antibody comprises a heavy chain variable region sequence having an amino acid sequence set forth in SEQ ID NO: 3. In certain embodiments, the antibody comprises a light chain variable region sequence having an amino acid sequence set forth in SEQ ID NO: 4.

In certain embodiments, the pharmaceutical compositions further comprise at least one inhibitor of glycosphingolipid synthesis comprises: D-threo-1-phenyl-2-d ecanoylamino-3-morpholino-1-propanol (D-PDMP), (1R,2R)-nonanoic acid(2-(2′,3-dihydro-benzo (1, 4) dioxin-6′-yl)-2-hydroxy-1-pyrrolidin-1-ylmethyl-ethyl)-amide-L-tartaric acid salt (Genz-123346), an imide sugar, 1-phenyl-2-decanoylamino-3-morpholino-1-propanol (DMP), 1-phenyl-2-palmitoyl-amino-3-morpholino-1-propanol (PPMP), lipids, ceramides or combinations thereof are unencapsulated or encapsulated by a biodegradable polymer. In certain embodiments, the inhibitor of glycosphingolipid synthesis is D-threo-1-phenyl-2-decanoylamino-3-morpholino-1-propanol (D-PDMP), unencapsulated or encapsulated in a biodegradable polymer (BPD). In certain embodiments, the biodegradable polymer consists of polyethylene glycol and sebacic acid.

In certain embodiments, the pharmaceutical compositions further comprise one or more secondary therapeutic agents. In certain embodiments, the one or more secondary therapeutic agents comprise: chemotherapeutic agents, anti-inflammatory agents, cholesterol lowering agents, insulin, antibodies, peptides, enzymes, adjuvants or combinations thereof. In certain embodiments, the pharmaceutical composition further comprises conjugating the antibody to a detectable agent, a radiotherapeutic agent, a toxin, a radioactive agent, a dye, a peptide, a polynucleotide or a nanoliposome. In certain embodiments, the nanoliposome comprises a therapeutic agent(s).

In certain embodiments, the pharmaceutical composition further comprises a peptide having at least a 90% sequence identity to IGAQVYEQVLRSAYAKRNSSVND (SEQ ID NO: 5).

In certain embodiments, the composition comprises a therapeutically effective amount of at least one inhibitor of glycosphingolipid synthesis and/or a therapeutically effective amount of the antibody which specifically binds to β1,4-Galactosyltransferase V (BGA), isoforms or peptides thereof.

In certain embodiments, the composition comprises a therapeutically effective amount of at least one inhibitor of glycosphingolipid synthesis and/or a therapeutically effective amount of an agent which modulates the expression or activity of β1,4-Galactosyltransferase V (BGA), isoforms or peptides thereof. In certain embodiments, the agent inhibits the expression or activity of β1,4-Galactosyltransferase V (BGA), isoforms or peptides thereof.

Examples of lipids include, without limitation fatty acids, free fatty acids, cholesterol, sterol esters, triglycerides, diglycerides, glycerides, wax esters, squalene, ceramides, lipids, phospholipids, glycolipids, linoleic acids or combinations thereof.

In other embodiments, a method of treating cancer, comprises administering to a subject in need thereof a therapeutically effective amount of an inhibitor of glycosphingolipid synthesis, lipids or combinations thereof. In certain embodiments, the inhibitor of glycosphingolipid synthesis is D-threo-1-phenyl-2-decanoylamino-3-morpholino-1-propanol (D-PDMP), unencapsulated, unbound or encapsulated in a biodegradable polymer (BPD). In certain embodiments, the biodegradable polymer consists of polyethylene glycol and sebacic acid.

### Combination Therapies

In certain embodiments, the pharmaceutical compositions include an anti-cancer agent, such as, a chemotherapeutic agent, radiotherapy, a toxin or combinations thereof.

In certain embodiments, In certain embodiments, the anti-cancer agent is a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof. In certain embodiments, the anti-cancer agent is a chemotherapeutic or growth inhibitory agent. For example, a chemotherapeutic or growth inhibitory agent may include an alkylating agent, an anthracycline, an anti-hormonal agent, an aromatase inhibitor, an anti-androgen, a protein kinase inhibitor, a lipid kinase inhibitor, an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof. In certain embodiments, the anti-cancer agent is an adjuvant. Any substance that enhances an anti-cancer immune response, such as against a cancer-related antigen, or aids in the presentation of a cancer antigen to a component of the immune system may be considered an anti-cancer adjuvant of the present disclosure.

Chemotherapy: Cancer therapies in general also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The chemotherapy may also be administered at low, continuous doses which is known as metronomic chemotherapy.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above. In certain embodiments, one or more chemotherapeutic may be used in combination with the compositions provided herein.

Radiotherapy: Other factors that cause DNA damage and have been used extensively in cancer therapies include what are commonly known as gamma-rays, X-rays, and/or the directed delivery of radioisotopes to tumor cells. Other forms of DNA damaging factors are also known such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Immunotherapy: Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells as well as genetically engineered variants of these cell types modified to express chimeric antigen receptors.

The immunotherapy may be a cancer vaccine comprising one or more cancer antigens, in particular a protein or an immunogenic fragment thereof, DNA or RNA encoding said cancer antigen, in particular a protein or an immunogenic fragment thereof, cancer cell lysates, and/or protein preparations from tumor cells. As used herein, a cancer antigen is an antigenic substance present in cancer cells. In principle, any protein produced in a cancer cell that has an abnormal structure due to mutation can act as a cancer antigen. In principle, cancer antigens can be products of mutated oncogenes and tumor suppressor genes, products of other mutated genes, overexpressed or aberrantly expressed cellular proteins, cancer antigens produced by oncogenic viruses, oncofetal antigens, altered cell surface glycolipids and glycoproteins, or cell type-specific differentiation antigens. Examples of cancer antigens include the abnormal products of ras and p53 genes. Other examples include tissue differentiation antigens, mutant protein antigens, oncogenic viral antigens, cancer-testis antigens and vascular or stromal specific antigens. Tissue differentiation antigens are those that are specific to a certain type of tissue. Mutant protein antigens are likely to be much more specific to cancer cells because normal cells shouldn't contain these proteins. Normal cells will display the normal protein antigen on their MHC molecules, whereas cancer cells will display the mutant version. Some viral proteins are implicated in forming cancer, and some viral antigens are also cancer antigens.

In certain embodiments, the method of treating cancer comprises administering a therapeutically effective amount of a synthetic peptide comprising an amino acid sequence having at least a 90% amino acid sequence to SEQ ID NO: 5 and at least one adjuvant. In certain embodiments, the synthetic peptide comprises SEQ ID NO: 5. Administration of a therapeutically effective amount of SEQ ID NO: 5 generates an immune response to β-1,4-galactosyltransferase-V (β-1,4-GalT-V) In certain embodiments, an adjuvant is also administered to the subject.

In certain embodiments, the immunotherapy may be an antibody, such as part of a polyclonal antibody preparation, or may be a monoclonal antibody. The antibody may be a humanized antibody, a chimeric antibody, an antibody fragment, a bispecific antibody or a single chain antibody. An antibody as disclosed herein includes an antibody fragment, such as, but not limited to, Fab, Fab′ and F(ab′)₂, Fd, single-chain Fvs (scFv), single-chain antibodies, disulfide-linked Fvs (sdfv) and fragments including either a VL or VH domain.

In certain embodiments, the antibody comprises: (i) a heavy chain variable region sequence having at least a 90% amino acid sequence identity to: EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 3), and, (ii) a light chain variable sequence having at least a 90% amino acid sequence identity to:

(SEQ ID NO: 4)
DVVMTQTPPTLSVTIGQPASISCKSSQSLLDSDGKTYLNWLLQRPGQSP
KRLIYLVSKLGSGVPDRFTGSGSGTDFTLKISRVEAEDLGVYYCWQGTH
FPRTFGGGTKLEIKR.

In certain embodiments, one or more antibodies are administered to a subject in need thereof as a combination therapy. Examples of monoclonal antibodies that may be used in combination with the compositions provided herein include, without limitation, trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-I131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgGI Fc); AMG386 (angiopoietin-1 and −2 binding peptide fused to IgGI Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™ (17-1A) (murine monoclonal antibody); Panorex (17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Examples of antibodies include those disclosed in U.S. Pat. Nos. 5,736,167, 7,060,808, and 5,821,337.

Passive Immunotherapy: A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Accordingly, in certain embodiments, a method of treating cancer, comprises administering to a subject in need thereof a composition comprising a therapeutically effective amount of: (a) an antibody, wherein the antibody specifically binds to a β-1,4-galactosyltransferase-V (β-1,4-GalT-V) epitope, the antibody comprising: (i) a heavy chain variable region sequence having at least a 90% amino acid sequence identity to: EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 3), and, (ii) a light chain variable sequence having at least a 90% amino acid sequence identity to: DVVMTQTPPTLSVTIGQPASISCKSSQSLLDSDGKTYLNWLLQRPGQSPKRLIYLVSK LGSGVPDRFTGSGSGTDFTLKISRVEAEDLGVYYCWQGTHFPRTFGGGTKLEIKR (SEQ ID NO: 4), and, one or more secondary therapeutic agents. In certain embodiments, the one or more secondary therapeutic agents comprise: chemotherapeutic agents, anti-inflammatory agents, cholesterol lowering agents, insulin, antibodies, peptides, enzymes, adjuvants or combinations thereof. In certain embodiments, the pharmaceutical composition further comprises conjugating the antibody to a detectable agent, a radiotherapeutic agent, a toxin, a radioactive agent, a dye, a peptide, a polynucleotide or a nanoliposome. In certain embodiments, the nanoliposome comprises a therapeutic agent(s).

Other Agents: It is contemplated that other agents may be used in combination with the compositions provided herein to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the compositions provided herein by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the compositions provided herein to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the compositions provided herein to improve the treatment efficacy.

In further embodiments, the other agents may be one or more oncolytic viruses, such as an oncolytic viruses engineered to express a gene other than p53 and/or IL24, such as a cytokine. Examples of oncolytic viruses include adenoviruses, adeno-associated viruses, retroviruses, lentiviruses, herpes viruses, pox viruses, vaccinia viruses, vesicular stomatitis viruses, polio viruses, Newcastle's Disease viruses, Epstein-Barr viruses, influenza viruses and reoviruses.

In certain embodiments, hormonal therapy may also be used in conjunction with the present embodiments or in combination with any other cancer therapy previously described. The use of hormones may be employed to lower the level or block the effects of certain hormones. This treatment is often used in combination with at least one other cancer therapy as a treatment option or to reduce the risk of metastases

In some aspects, the additional anti-cancer agent is a protein kinase inhibitor or a monoclonal antibody that inhibits receptors involved in protein kinase or growth factor signaling pathways such as an EGFR, VEGFR, AKT, Erb1, Erb2, ErbB, Syk, Bcr-Abl, JAK, Src, GSK-3, PI3K, Ras, Raf, MAPK, MAPKK, mTOR, c-Kit, eph receptor or BRAF inhibitors. Nonlimiting examples of protein kinase or growth factor signaling pathways inhibitors include Afatinib, Axitinib, Bevacizumab, Bosutinib, Cetuximab, Crizotinib, Dasatinib, Erlotinib, Fostamatinib, Gefitinib, Imatinib, Lapatinib, Lenvatinib, Mubritinib, Nilotinib, Panitumumab, Pazopanib, Pegaptanib, Ranibizumab, Ruxolitinib, Saracatinib, Sorafenib, Sunitinib, Trastuzumab, Vandetanib, AP23451, Vemurafenib, MK-2206, GSK690693, A-443654, VQD-002, Miltefosine, Perifosine, CAL101, PX-866, LY294002, rapamycin, temsirolimus, everolimus, ridaforolimus, Alvocidib, Genistein, Selumetinib, AZD-6244, Vatalanib, P1446A-05, AG-024322, ZD1839, P276-00, GW572016 or a mixture thereof.

It is contemplated that the additional cancer therapy can comprise an antibody, peptide, polypeptide, small molecule inhibitor, siRNA, miRNA or gene therapy which targets, for example, epidermal growth factor receptor (EGFR, EGFR1, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family (IGF-1R); platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family; AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family; MuSK receptor family; Transforming growth factor alpha (TGF-α), TGF-α receptor; Transforming growth factor-beta (TGF-β), TGF-β receptor; Interleukin 13 receptor alpha2 chain (1L13Ralpha2), Interleukin-6 (IL-6), 1L-6 receptor, Interleukin-4, IL-4 receptor, Cytokine receptors, Class I (hematopoietin family) and Class II (interferon/IL-10 family) receptors, tumor necrosis factor (TNF) family, TNF-α, tumor necrosis factor (TNF) receptor superfamily (TNTRSF), death receptor family, TRAIL-receptor; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), beta-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: beta-Dgalactose 2-alpha-Lfucosyltraosferase (LDLR/FUT) fusion protein, HLA-A2, arginine to isoleucine exchange at residue 170 of the alpha-helix of the alpha2-domain in the HLA-A2 gene (HLA-A*201-R170I), MLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDXS, PTPRK, K-ras (KRAS2), N-ras (NRAS), HRAS, RBAF600, SIRT2, SNRPD1, SYT-SSX1 or -SSX2 fusion protein, Triosephosphate Isomerase, BAGE, BAGE-1, BAGE-2, 3, 4, 5, GAGE-1, 2, 3, 4, 5, 6, 7, 8, GnT-V (aberrant N-acetyl giucosaminyl transferase V, MGATS), HERV-K-MEL, KK-LC, KM-HN-1, LAGE, LAGE-1, CTL-recognixed antigen on melanoma (CAMEL), MAGE-A1 (MAGE-1), MAGE-A2, MAGE-A3, MAGE-A4, MAGE-AS, MAGE-A6, MAGE-A8, MAGE-A9, MAGE-A10, MAGE-A11, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gp100, gp100/Pme117 (S1LV), tyrosinase (TYR), TRP-1, HAGE, NA-88, NY-ESO-1, NY-ESO-1/LAGE-2, SAGE, Sp17, SSX-1, 2, 3, 4, TRP2-1NT2, carcino-embryonic antigen (CEA), Kallikfein 4, mammaglobm-A, OA1, prostate specific antigen (PSA), prostate specific membrane antigen, TRP-1/gp75, TRP-2, adipophilin, interferon inducible protein absent in nielanorna 2 (AIM-2), BING-4, CPSF, cyclin D1, epithelial cell adhesion molecule (Ep-CAM), EpbA3, fibroblast growth factor-5 (FGF-5), glycoprotein 250 (gp250intestinal carboxyl esterase (iCE), alpha-feto protein (AFP), M-CSF, mdm-2, MUCI, p53 (TP53), PBF, FRAME, PSMA, RAGE-1, RNF43, RU2AS, SOX10, STEAP11, survivin (BIRCS), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SPO11, TPX1, NY-SAR-35, FTHLI7, NXF2 TDRD1, TEX 15, FATE, TPTE, immunoglobulin idiotypes, Bence-Jones protein, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, neuron-specific enoJase, heat shock protein gp96, GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr vims (EBV) proteins (EBV latent membrane proteins-LMP1, LMP2; others), Hepatitis B or C virus proteins, and HIV proteins

Glycosphingolipid Synthesis Inhibitors: In certain embodiments, the method further comprises administering at least one inhibitor of glycosphingolipid synthesis comprising: D-threo-1-phenyl-2-decanoylamino-3-morpholino-1-propanol (D-PDMP), (1R,2R)-nonanoic acid(2-(2′,3′-dihydro-benzo (1, 4) dioxin-6′-yl)-2-hydroxy-1-pyrrolidin-1-ylmethyl-ethyl)-amide-L-tartaric acid salt (Genz-123346), an imide sugar, 1-phenyl-2-decanoylamino-3-morpholino-1-propanol (DMP), 1-phenyl-2-palmitoyl-amino-3-morpholino-1-propanol (PPMP), lipids, ceramides or combinations thereof are unencapsulated or encapsulated by a biodegradable polymer. In certain embodiments, the inhibitor of glycosphingolipid synthesis is D-threo-1-phenyl-2-decanoylamino-3-morpholino-1-propanol (D-PDMP), including D-PDMP that may be admixed with a biodegradable polymer e.g. unencapsulated or encapsulated in a biodegradable polymer (BPD). In certain embodiments, the biodegradable polymer consists of polyethylene glycol and sebacic acid.

In certain embodiments, a composition comprises an inhibitor of glycosphingolipid synthesis, an inhibitor of glucosylceramide synthase or a combination thereof. In certain embodiments, a compound that inhibits glucosylceramide synthesis is an imino sugar. In another embodiment, the imide sugar is N-butyldeoxynojirimycin, N-butyldeoxygalactonojirimycin (NB-DGJ), or N-nonyldeoxynojirimycin. In another embodiment, the inhibitor of glucosylceramide synthesis is 1-phenyl-2-decanoylamino-3-morpholino-1-propanol (DMP), D-threo-1-phenyl-2-decanoylamino-3-morpholino-1-propanol and structurally related analogues thereof. In another embodiment, the inhibitor of glucosylceramide synthesis is 1-phenyl-2-palmitoyl-amino-3-morpholino-1-propanol (PPMP) and structurally related analogues thereof. In certain embodiments, the composition comprises D-threo-1-phenyl-2-decanoylamino-3-morpholino-1-propanol (D-PDMP), (1R,2R)-nonanoic acid(2-(2′,3′-dihydro-benzo (1, 4) dioxin-6′-yl)-2-hydroxy-1-pyrrolidin-1-ylmethyl-ethyl)-amide-L-tartaric acid salt (Genz-123346), an imide sugar, 1-phenyl-2-decanoylamino-3-morpholino-1-propanol (DMP), 1-phenyl-2-palmitoyl-amino-3-morpholino-1-propanol (PPMP), lipids, ceramides or combinations thereof are encapsulated by a biodegradable polymer.

### Pharmaceutical Formulations

In certain embodiments, the pharmaceutical compositions embodied herein are formulated for systemic administration, e.g. oral, i.v., i.m. etc., comprises a therapeutically effective amount of inhibitor of glycosphingolipid synthesis, such as, a therapeutically effective amount of: (a) an antibody, wherein the antibody specifically binds to a β-1,4-galactosyltransferase-V (β-1,4-GalT-V) epitope, the antibody comprising: (i) a heavy chain variable region sequence having at least a 90% amino acid sequence identity to: EVQLEQSGAELARPGASVKLSCRTSGYTFTNYWMQWIKQRPGQGLEWIGAMHPGR AYIRYNQKFQGKATLTADKSSSTAYMQLNSLASEDSAVYYCARWSDYDYWGQGTT LTVSS (SEQ ID NO: 3), and, (ii) a light chain variable sequence having at least a 90% amino acid sequence identity to: DVVMTQTPPTLSVTIGQPASISCKSSQSLLDSDGKTYLNWLLQRPGQSPKRLIYLVSK LGSGVPDRFTGSGSGTDFTLKISRVEAEDLGVYYCWQGTHFPRTFGGGTKLEIKR (SEQ ID NO: 4), and/or a peptide comprising SEQ ID NO: 5 and/or for example, D-threo-1-phenyl-2-decanoylamino-3-morpholino-1-propanol (D-PDMP) including D-PDMP that may be admixed with a biodegradable polymer e.g. unencapsulated or encapsulated in a biodegradable polymer (BPD), or combinations thereof.

The pharmaceutical compositions may include a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, olive oil, gel (e.g., hydrogel), and the like. Saline is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin, the contents of which are hereby incorporated by reference in its entirety. Such compositions will generally contain a therapeutically effective amount of the pharmaceutical agents and/or therapeutic compounds (e.g., biopolymer encapsulated D-PDMP), in purified form, together with a suitable amount of carrier so as to provide the form for proper administration to the patient. The formulation should suit the mode of administration.

In embodiments, the pharmaceutical agents and/or therapeutic compounds are administered locally as an immediate release or controlled release composition, for example by controlled dissolution and/or the diffusion of the active substance. Dissolution or diffusion controlled release can be achieved by incorporating the active substance into an appropriate matrix. A controlled release matrix may include one or more of a biopolymer, shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols and/or sebacic acid. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon. In certain embodiments, the controlled release composition is achieved via a transdermal patch.

The controlled release matrix may also be a hydrogel: a three-dimensional, hydrophilic or amphiphilic polymeric network capable of taking up large quantities of water. The networks may be composed of homopolymers or copolymers, which are insoluble due to the presence of covalent chemical or physical (e.g., ionic, hydrophobic interactions, entanglements) crosslinks. The crosslinks provide the network structure and physical integrity. Hydrogels exhibit a thermodynamic compatibility with water that allows them to swell in aqueous media. The chains of the network are connected in such a fashion that pores exist and that a substantial fraction of these pores are of dimensions between 1 nm and 1000 nm.

The hydrogels can be prepared by crosslinking hydrophilic biopolymers or synthetic polymers. Examples of the hydrogels formed from physical or chemical crosslinking of hydrophilic biopolymers, include but are not limited to, hyaluronans, chitosans, alginates, collagen, dextran, pectin, carrageenan, polylysine, gelatin, agarose, (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, poly(ethylene imine), and the like. See Hennink and van Nostrum, Adv. Drug Del. Rev. 54:13-36 (2002); Hoffman, Adv. Drug Del. Rev. 43:3-12 (2002); Cadee et al., J Control. Release 78:1-13 (2002); Surini et al., J Control. Release 90:291-301 (2003); and U.S. Pat. No. 7,968,085, each of which is incorporated by reference in its entirety. These materials consist of high-molecular weight backbone chains made of linear or branched polysaccharides or polypeptides.

The amount of the pharmaceutical composition of the invention which will be effective in the treatment or prevention of atherosclerotic heart disease can be determined by standard clinical techniques. In addition, in vitro assays may optionally be employed to help identify optimal dosage ranges. The precise dose to be employed in the formulation may also depend on the route of administration, and the seriousness of the disease, and should be decided according to the judgment of the practitioner and each patient's circumstances. Effective doses may be extrapolated from dose-response curves derived from the in vitro or animal model test systems described herein or known to one of skill in the art.

### Dosages and Administration Regimens

The pharmaceutical agents and/or therapeutic compounds or compositions containing these agents/compounds may be administered in a manner compatible with the dosage formulation, and in such amount as may be therapeutically affective, protective and immunogenic.

The agents and/or compositions may be administered through different routes, including, but not limited to, oral, oral gavage, parenteral, buccal and sublingual, rectal, aerosol, nasal, intramuscular, subcutaneous, intradermal, intraosseous, dermal, and topical. The term parenteral as used herein includes, for example, intraocular, subcutaneous, intraperitoneal, intracutaneous, intravenous, intramuscular, intraarticular, intraarterial, intrasynovial, intrastemal, intrathecal, intralesional, and intracranial injection, or other infusion techniques.

In embodiments, the pharmaceutical agents and/or therapeutic compounds formulated according to the present invention are formulated and delivered in a manner to evoke a systemic response. Thus, in embodiments, the formulations are prepared by uniformly and intimately bringing into association the active ingredient with liquid carriers. Formulations suitable for administration include aqueous and non-aqueous sterile solutions, which may contain anti-oxidants, buffers, bacteriostats and solutes which render the formulation isotonic with the blood of the intended recipient, and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water, immediately prior to use. Extemporaneous solutions and suspensions may be prepared from sterile powders, granules and tablets commonly used by one of ordinary skill in the art.

The agents and/or compositions may be administered in different forms, including, but not limited to, solutions, emulsions and suspensions, microspheres, particles, microparticles, nanoparticles, liposomes, and the like.

The pharmaceutical agents and/or therapeutic compounds may be administered in a manner compatible with the dosage formulation, and in such amount as may be therapeutically effective, immunogenic and protective. The quantity to be administered depends on the subject to be treated, including, for example, the stage of the disease. Precise amounts of active ingredients required to be administered depend on the judgment of the practitioner. However, suitable dosage ranges are readily determinable by one skilled in the art and may be of the order of micrograms to milligrams of the active ingredient(s) per dose. The dosage may also depend on the route of administration and may vary according to the size of the host.

The pharmaceutical agents and/or therapeutic compounds should be administered to a subject in an amount effective to ameliorate, treat, and/or prevent the disease. Specific dosage and treatment regimens for any particular subject may depend upon a variety of factors, including the activity of the specific compound employed, the age, body weight, general health status, sex, diet, time of administration, rate of excretion, drug combination, the severity and course of the disease (including tumor size), condition or symptoms, the subject's disposition to the disease, condition or symptoms, method of administration, and the judgment of the treating physician. Actual dosages can be readily determined by one of ordinary skill in the art.

Exemplary unit dosage formulations are those containing a dose or unit, or an appropriate fraction thereof, of the administered ingredient. It should be understood that in addition to the ingredients mentioned herein, the formulations of the present invention may include other agents commonly used by one of ordinary skill in the art.

In certain embodiments, the antibody which specifically binds to β1,4-Galactosyltransferase V (BGA), isoforms or peptides thereof is administered systemically or via endoscopy or intra-anally.

In certain embodiments, the composition comprising a therapeutically effective amount of at least one inhibitor of glycosphingolipid synthesis and/or a therapeutically effective amount of an agent which modulates the expression or activity of β1,4-Galactosyltransferase V (BGA), isoforms or peptides thereof is administered systemically or topically.

In certain embodiments, the composition comprising a therapeutically effective amount of at least one inhibitor of glycosphingolipid synthesis and/or a therapeutically effective amount of an agent which modulates the expression or activity of β1,4-Galactosyltransferase V (BGA), isoforms or peptides thereof is co-administered to the subject. The term “co-administer” refers to the simultaneous presence of two active agents in the blood of an individual. Active agents that are co-administered can be concurrently or sequentially delivered.

Typically in conventional systemically administered treatments, a therapeutically effective dosage should produce a serum concentration of compound of from about 0.1 ng/ml to about 50-100 μg/ml. The pharmaceutical compositions typically provide a dosage of from about 0.001 mg to about 2000 mg of compound per kilogram of body weight per day. For example, dosages for systemic administration to a human patient can range from 1-10 μg/kg, 20-80 μg/kg, 5-50 μg/kg, 75-150 μg/kg, 100-500 μg/kg, 250-750 μg/kg, 500-1000 μg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 1500-2000 mg/kg, 5 mg/kg, 20 mg/kg, 50 mg/kg, 100 mg/kg, 500 mg/kg, 1000 mg/kg, 1500 mg/kg, or 2000 mg/kg. In an exemplary embodiment, an oral dosage for a human weighing 200 kg would be about 200 mg/day. Pharmaceutical dosage unit forms are prepared to provide from about 1 mg to about 5000 mg, for example from about 100 to about 2500 mg of the compound or a combination of essential ingredients per dosage unit form.

In general, a therapeutically effective amount of the present compounds in dosage form usually ranges from slightly less than about 0.025 mg/kg/day to about 2.5 g/kg/day, preferably about 0.1 mg/kg/day to about 100 mg/kg/day of the patient or considerably more, depending upon the compound used, the condition or infection treated and the route of administration, although exceptions to this dosage range may be contemplated by the present invention. It is to be understood that the present invention has application for both human and veterinary use.

The agents and/or compositions are administered in one or more doses as required to achieve the desired effect. Thus, the agents and/or compositions may be administered in 1, 2, to 3, 4, 5, or more doses. Further, the doses may be separated by any period of time, for example hours, days, weeks, months, and years.

The agents and/or compositions can be formulated as liquids or dry powders, or in the form of microspheres.

The agents and/or compositions may be stored at temperatures of from about −100° C. to about 25° C. depending on the duration of storage. The agents and/or compositions may also be stored in a lyophilized state at different temperatures including room temperature. The agents and/or compositions may be sterilized through conventional means known to one of ordinary skill in the art. Such means include, but are not limited to, filtration. The composition may also be combined with other anti-atherosclerotic therapeutic agents.

The amount of active ingredient that may be combined with carrier materials to produce a single dosage form may vary depending upon the host treated and the particular mode of administration. In embodiments, a preparation may contain from about 0.1% to about 95% active compound (w/w), from about 20% to about 80% active compound, or from any percentage therebetween.

In embodiments, the pH of the formulation may be adjusted with pharmaceutically acceptable acids, bases, or buffers to enhance the stability of the formulated compound or its delivery form.

In embodiments, the pharmaceutical carriers may be in the form of a sterile liquid preparation, for example, as a sterile aqueous or oleaginous suspension. Among the acceptable vehicles and solvents that may be employed are mannitol, water, Ringer's solution and isotonic sodium chloride solution.

In addition, sterile, fixed oils are conventionally employed as a solvent or suspending medium. For this purpose, any bland fixed oil may be employed including synthetic mono- or to diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions.

Other commonly used surfactants such as TWEEN™ or SPAN™ and/or other similar emulsifying agents or bioavailability enhancers which are commonly used in the manufacture of pharmaceutically acceptable solid, liquid, or other dosage forms may also be used for the purposes of formulation.

In embodiments, the agents and/or compositions can be delivered in an exosomal delivery system. Exosomes are small membrane vesicles that are released into the extracellular environment during fusion of multivesicular bodies with plasma membrane. Exosomes are secreted by various cell types including hematopoietic cells, normal epithelial cells and even some tumor cells.

In certain embodiments, the biopolymer encapsulating the D-PDMP comprises polyethelene glycol (PEG) and sebacic acid (SA). Both PEG and SA are FDA approved. The polyethylene glycol-sebacic acid (PEG-SA) copolymer can be prepared as previously described (Fu J, et al. Biomaterials. 2002; 23:4425-4433), Microparticles of D-PDMP encapsulated by the PEG-SA copolymer are prepared by modifying the single emulsion solvent evaporation method. For scintigraphic tracking of the biopolymer, the PEG polymer is radio-iodinated with 45 mCi (810 kBq) of (¹²⁵I)NaI. The radiolabeled PEG was then incorporated into the PEG-SA biopolymer. The PEG-SA co-polymer can be prepared following the published literature procedure by Fu and coworkers (Id.). Briefly, sebacic acid prepolymer is made by refluxing sebacic acid (SA) in acetic anhydride followed by dryingunder high vacuum (evaporation), crystallized from dry toluene, washed with 1:1 anhydrous ethyl ether-petroleum ether and finally air dried. PEG prepolymer is made by refluxing of polyoxyethylene dicarboxylic acid in acetic anhydride, volatile solvents are removed under vacuum. The solid mass is extracted with anhydrous ether and air dried. The poly(PEG-SA) co-block polymer is then synthesized by the melt polycondensation method and characterized by proton NMR. Note that this copolymer has been extensively characterized for the composition and structural identity (Aich U, et al. Glycoconjugate journal. 2010; 27: 445-459).

Encapsulation of D-PDMP in poly(PEG-SA) (to prepare polymer-encapsulated drug subsequently referred to as BPD) followed by the melt polycondensation method described above for SA and PEG prepolymers but with the inclusion of D-PDMP at starting ratios of poly (PEG-SA) to D-PDMP of 70:30 by weight. Subsequently, microparticles are prepared using a single emulsion solvent evaporation method. Briefly, D-PDMP and PEG-SA are dissolved in chloroform (50 mg/mL) and emulsified into a 1.0% w/w poly(vinyl alcohol) aqueous solution under sonication condition keeping the temperature below 25° C. Particles are hardened by allowing chloroform to evaporate at room temperature while stirring for 12 h. Particles are collected and washed three times with double distilled water via centrifugation at 2,600.times.g (30 min) and lyophilized for 48 h before it was ready to use.

In certain embodiments, the D-PDMP is encapsulated in a multilamellar lipid vesicle comprising covalent crosslinks between lipid bilayers, wherein at least two lipid bilayers in the multilamellar lipid vesicle are covalently crosslinked to each other by a thiolated biopolymer. In certain embodiments, the lipid bilayers are crosslinked via functionalized lipids. In certain embodiments, the one or more lipids comprise DOTAP, DOPE, DOBAQ, DOPC or combinations thereof. In certain embodiments, the lipid is maleimide-functionalized or modified with dibenzocyclooctyne (DBCO). In certain embodiments, the thiolated biopolymer is selected from the group consisting of chitosan, polyglutamic acid, polyphosphazene, polyethyleneimine, polyalky acrylic acids (e.g. polymethylmethacrylate, poly(ethylacrylic acid), poly(propylacrylic acid), or poly(butylacrylic acid), HA, pegylated azide-modified polyethylenimine, branched polyethylenimine, and diazide. In certain embodiments, the thiolated biopolymer comprises multiple sulfhydryl moieties.

Also contemplated by the invention is delivery of the pharmaceutical agents and/or therapeutic compounds using nanoparticles. For example, the agents and/or compositions provided herein can contain nanoparticles having at least one or more agents linked thereto, e.g., linked to the surface of the nanoparticle. A composition typically includes many nanoparticles with each nanoparticle having at least one or more agents linked thereto. Nanoparticles can be colloidal metals. A colloidal metal includes any water-insoluble metal particle or metallic compound dispersed in liquid water. Typically, a colloid metal is a suspension of metal particles in aqueous solution. Any metal that can be made in colloidal form can be used, including gold, silver, copper, nickel, aluminum, zinc, calcium, platinum, palladium, and iron. In some cases, gold nanoparticles are used, e.g., prepared from HAuCl₄. Nanoparticles can be any shape and can range in size from about 1 nm to about 10 nm in size, e.g., about 2 nm to about 8 nm, about 4 to about 6 nm, or about 5 nm in size. Methods for making colloidal metal nanoparticles, including gold colloidal nanoparticles from HAuCl₄, are known to those having ordinary skill in the art. For example, the methods described herein as well as those described elsewhere (e.g., US Pat. Publication Nos. 2001/005581; 2003/0118657; and 2003/0053983, which are hereby incorporated by reference) are useful guidance to make nanoparticles.

In certain cases, a nanoparticle can have two, three, four, five, six, or more active agents linked to its surface. Typically, many molecules of active agents are linked to the surface of the nanoparticle at many locations. Accordingly, when a nanoparticle is described as having, for example, two active agents linked to it, the nanoparticle has two active agents, each having its own unique molecular structure, linked to its surface. In some cases, one molecule of an active agent can be linked to the nanoparticle via a single attachment site or via multiple attachment sites.

An active agent can be linked directly or indirectly to a nanoparticle surface. For example, the active agent can be linked directly to the surface of a nanoparticle or indirectly through an intervening linker.

Any type of molecule can be used as a linker. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide to functionalities. In cases where the nanoparticle includes gold, a linker can be any thiol-containing molecule. Reaction of a thiol group with the gold results in a covalent sulfide (—S—) bond. Linker design and synthesis are well known in the art.

In embodiments, the nanoparticle is linked to a targeting agent/moiety. A targeting functionality can allow nanoparticles to accumulate at the target at higher concentrations than in other tissues. In general, a targeting molecule can be one member of a binding pair that exhibits affinity and specificity for a second member of a binding pair. For example, an antibody or antibody fragment therapeutic agent can target a nanoparticle to a particular region or molecule of the body (e.g., the region or molecule for which the antibody is specific) while also performing a therapeutic function. In some cases, a receptor or receptor fragment can target a nanoparticle to a particular region of the body, e.g., the location of its binding pair member. Other therapeutic agents such as small molecules can similarly target a nanoparticle to a receptor, protein, or other binding site having affinity for the therapeutic agent.

When the compositions of this invention comprise one or more additional therapeutic or prophylactic agents, the therapeutic agent and the additional agent should be present at dosage levels of between about 0.1 to 100%, or between about 5 to 95% of the dosage normally administered in a monotherapy regimen. The additional agents may be administered separately, as part of a multiple dose regimen, from the agents of this invention. Alternatively, those additional agents may be part of a single dosage form, mixed together with the agents of this invention in a single composition.

The administration of the pharmaceutical agents and/or therapeutic compounds of the invention elicits, for example, an anti-cancer response. Typically, the dose can be adjusted within this range based on, e.g., the subject's age, the subject's health and physical condition, the capacity of the subject's immune system to produce an immune response, the subject's body weight, the subject's sex, diet, time of administration, the degree of protection desired, and other clinical factors. Those in the art can also readily address parameters such as biological half-life, bioavailability, route of administration, and toxicity when formulating the agents and/or compositions of the invention.

### EXAMPLES

### Example 1: Lactosylceramide Synthase β-1,4-GalT-V: A Novel Target for the Diagnosis and Therapy of Human Colorectal Cancer

It was hypothesized that β-1,4-galactosyltransferase-V (β-1,4-GalT-V) may well play a role in human CRC, and that its inhibition may also mitigate tumor cell proliferation. To test this hypothesis, we acquired colorectal tissue samples from de-identified cancer patients and evaluated their immunoreactivity to β-1,4-GalT-V antibodies. In addition, β-1,4-GalT-V mass, mRNA expression, enzymatic activity, and GSL end-product levels were assessed. Finally, the effect of a GSL glycosyltransferase inhibitor in human CRC cell lines was also examined. These results provide new insights into the pathogenesis of and reveal promising detection/prognostic biomarkers for CRC. Furthermore, these findings demonstrate viable targets for future CRC therapeutics.

### Materials and Methods

### Immunohistochemical (IHC) Localization of β-1,4-GalT-V in Human CRC Tissue.

IHC was performed on archival tissue from The Johns Hopkins Pathology Department after approval from the Institutional Review Board on human subjects research. Archival tissue was sectioned from formalin fixed, paraffin embedded blocks of colon cancer cases selected from 2-3-year-old material. Four-micron thick sections were cut and stained for IHC analysis. β-1,4-GalT-V staining was performed on automated instruments using standard IHC methods. Briefly, sections were de-paraffinized, hydrated, and prepared for staining.

Sections were incubated with a β-1,4-GalT-V mouse monoclonal antibody raised against a GalT-V synthetic peptide, IGAQVYEQVLRSAYAKRNSSVND, SEQ ID NO: 5 (1:600 dilution) for 30 minutes. A secondary antibody anti-rabbit HRP was applied and a brown signal was developed using DAB chromogen detection (Cat #DS9800, Leica Biosystems). Slides were then counterstained with hematoxylin, washed, dehydrated, and cover slipped. Evaluation of IHC staining was done by a blinded pathologist. Stains were scored based on intensity of staining and area of tumor stained.

### H-Score Determination of Human CRC Tissue.

Tumor tissues were independently examined by two pathologists on the team, (RM and MA). Twenty-one cases of colon adenocarcinoma were selected from the Johns Hopkins Hospital archival data. Only colon cancer cases with primary origin were included for this study. For case selection, archival cases were reviewed and selected based on reports from anatomic pathologists. Unstained sections were obtained from the paraffin blocks and IHC with a GalT-V antibody was performed as described in the methods section. Results were evaluated after generating a scoring system of β-3, where 0=no staining, 1=very weak staining, 2=moderate staining, and 3=strong staining. Area of positive staining on the tumor was also assessed, which was used for H-score calculation.

### Gene Expression Analysis.

Patient normal and CRC tissues were provided through collaboration with Dr. Bert Vogelstein (JHU). Total RNA was isolated from tissue samples using RNAqueous-4PCR Kits (Life Technologies), per the manufacturer's instructions. TaqMan Gene Expression Assays (Applied Biosystems) were used to determine expression levels of adenomatous polyposis coli (APC, Hs01568269), N-myristoyltransferase-1 (NMT1, Hs00221506), tumor protein p53 (TP53, Hs01034249), UDP-Gal:βGlcNAc β-1,4-galactosyltransferase, polypeptide 5 (β-1,4-GalT-V, Hs00191142), UDP-Gal:βGlcNAc, β-1,4-galactosyltransferase, polypeptide 6 (β-1,4 GalT-VI, Hs00191135), and UDP-glucose ceramide glucosyltransferase (UGCG, Hs00234293). cDNA was synthesized from isolated RNA using the High Capacity cDNA Reverse Transcription Kit (Life Technologies 4374966), per the manufacturer's protocol. TaqMan gene expression assays were performed by 7900HT Fast Real-Time PCR at The Genetic Resources Core Facility (Johns Hopkins Medical Institutions).

### Measurement of Lactosylceramide Synthase (LCS) Activity.

LCS activity, in visibly normal and cancer tissues, was measured according to the inventor's previously published method (20, 21). All assays, for both 10 normal and 10 tumor samples, were run in triplicate, average values±standard error of measurements (SEm) represented, and an unpaired t-test conducted to determine statistical significance.

### Measurement of β-1,4-GalT-V Mass in CRC Tissue.

About 10 mg of tissue was homogenized in radioimmunoprecipitation assay (RIPA) buffer and centrifuged at 10,000 rpm. GalT-V mass was measured in the supernatant using ELISA, as previously published (22).

### Liquid Chromatography-Mass Spectrometry (LC-MS).

Sphingolipid levels in human CRC tissue and in human cultured CRC cells (HCT-116) were measured by LC-MS, as described previously (23). Visibly normal and CRC tissues (50 mg) were homogenized in chloroform-methanol (2:1), in the presence of sphingolipid internal standards. 105 HCT-116 cells were grown in 100 mm² sterile, plastic Petri dishes. Lipid extracts were subject to LC-MS, as described (24, 25).

### Determination of the Effect of D-PDMP on IHC Localization of Glycosyltransferases in Human CRC Cells.

HCT-116 cells were seeded (10⁴) onto sterilized glass coverslips, which were then placed in 6-well sterile plastic trays and grown for 24 h in complete media. Next, the media was replaced with 2 mL of 2% serum-containing media, plus 10p M D-PDMP. After incubation for 24 and 96 h, the media was removed, and cells were fixed with ethanol, washed, incubated with antibody against β-1,4-GalT-V or UGCG, and photographed.

### Determination of the Effect of D-PDMP on Cell Proliferation.

HCT-116 cells were seeded (10⁴/well) in 96-well sterile plastic plates, and grown for 24 h in complete medium, with 10% fetal calf serum. The media was then replaced with 2% serum-containing media (100 μL) plus ³H-thymidine (5 Ci/mL), with and without D-PDMP. After another 24 h incubation, incorporation of ³H-thymidine into DNA was measured by scintillation spectrometry.

### Results

### Human CRC Tissues Exhibit Strongly Positive Immunoreactivity to a β-1,4-GalT-V Antibody.

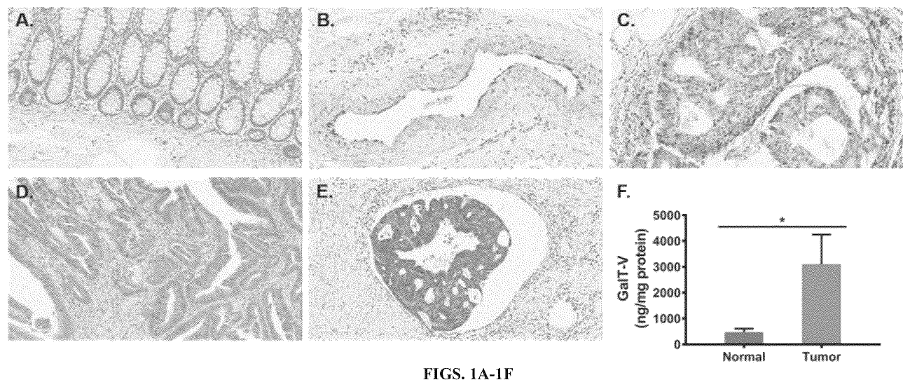

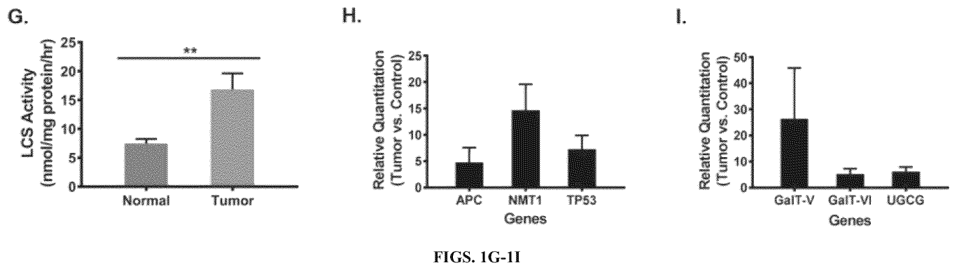

First, β-1,4-GalT-V immunoreactivity in 24 human CRC specimens was examined, using a monoclonal antibody. Normal colon tissue showed cytoplasmic localization of β-1,4-GalT-V and strongly positive immunostained endothelial cells in large and small blood vessels (FIG. 1B). In colonic adenocarcinomas (FIG. 1C), it was found that about 50% of lesioned cells expressed mild cytoplasmic β-1,4-GalT-V immunoreactivity. H-score analysis yielded a total score of 100 in some cases but reached that of 200 in some cases (FIG. 1D) and that of 300 in other cases (FIG. 1E), (Table 1). Additional studies showed strong immunoreactivity in perinuclear areas, in association with antigen localization to the Golgi apparatus, cytoplasm, and to a lesser extent, the inner aspect of the cell surface.

TABLE 1
β-1,4-GalT-V immunohistochemistry staining of tumor tissues.

| Case | % tumor score | | | Normal | H | Staining pattern |
|---|---|---|---|---|---|---|
| No | 1 | 2 | 3 | tissue | Score | Nuclear/Cytoplasmic |
| 1 | | 50 | | 0 | 120 | Cytoplasmic |
| 2 | 50 | | | 0 | 50 | Cytoplasmic |
| 3 | | 50 | | 1 | 100 | Cytoplasmic |
| 4 | | | 100 | 2 | 300 | Cytoplasmic |
| 5 | | 20 | | 1 | 40 | Cytoplasmic |
| 6 | | 80 | | 2 | 160 | Nuclear/Cytoplasmic |
| 7 | | 80 | | 2 | 160 | Cytoplasmic |
| 8 | | 90 | | 2 | 180 | Cytoplasmic |
| 9 | | | 100 | 1 | 300 | Cytoplasmic |
| 10 | | 60 | | 2 | 120 | Nuclear/Cytoplasmic |
| 11 | | 80 | | 2 | 160 | Cytoplasmic |
| 12 | 20 | | | 1 | 20 | Cytoplasmic |
| 13 | | | 90 | 0 | 270 | Cytoplasmic |
| 14 | | 90 | | 2 | 180 | Cytoplasmic |
| 15 | 30 | | | na | 30 | Nuclear/Cytoplasmic |
| 16 | | 30 | | 1 | 60 | Cytoplasmic |
| 17 | | 60 | | 2 | 240 | Cytoplasmic |
| 18 | | | 90 | 0 | 270 | Cytoplasmic |
| 19 | | 90 | | 1 | 180 | Nuclear/Cytoplasmic |
| 20 | | 85 | | 0 | 170 | Cytoplasmic |

H score is calculated as % tumor intensity score × total area of tumor

Table 1 shows the assessments for β-1,4-GalT-V immunohistochemistry staining of normal and CRC tissue sections. Review of the immuno-stains for GalT-V revealed varying degrees of positivity in the tumor cells: 1+(15%; 3/20), 2+(65%; 13/20), and 3+(20%; 4/20). Staining was mostly observed in the cytoplasm of tumors and very occasionally in the nuclei of tumor cells. Weak (1+) to moderate (2+) cytoplasmic staining was also observed in adjoining normal colonic mucosa in 79% cases (15/19), when available. In most cases normal colonic mucosa also stained at mild to moderate levels. The H score was determined as % tumor intensity score X total area of tumor.

CRC Tissues have Increased Protein Mass, Activity (Synthesis of Lactosylceramide), and Gene Expression of/β-1,4-GalT-V.

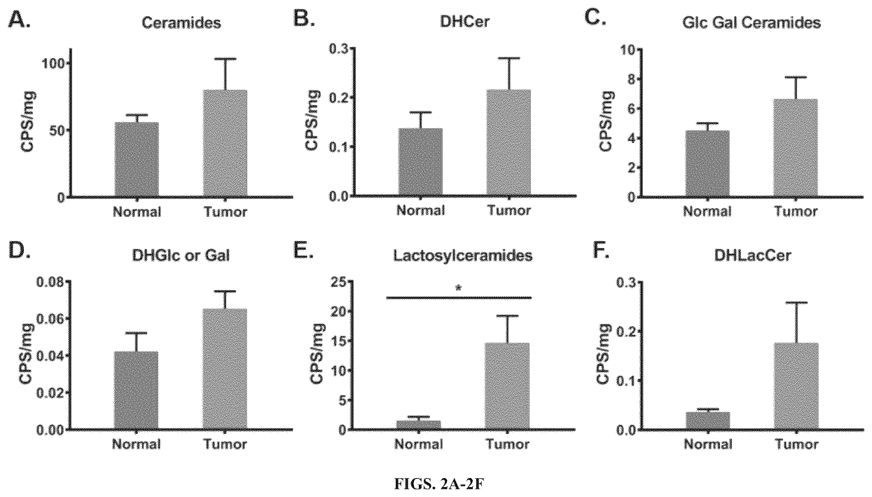

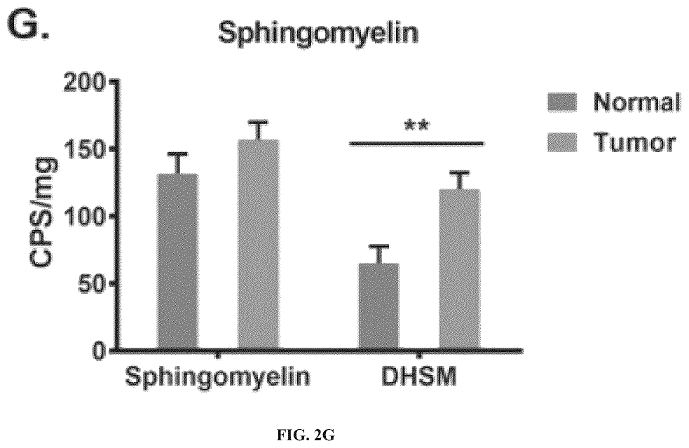

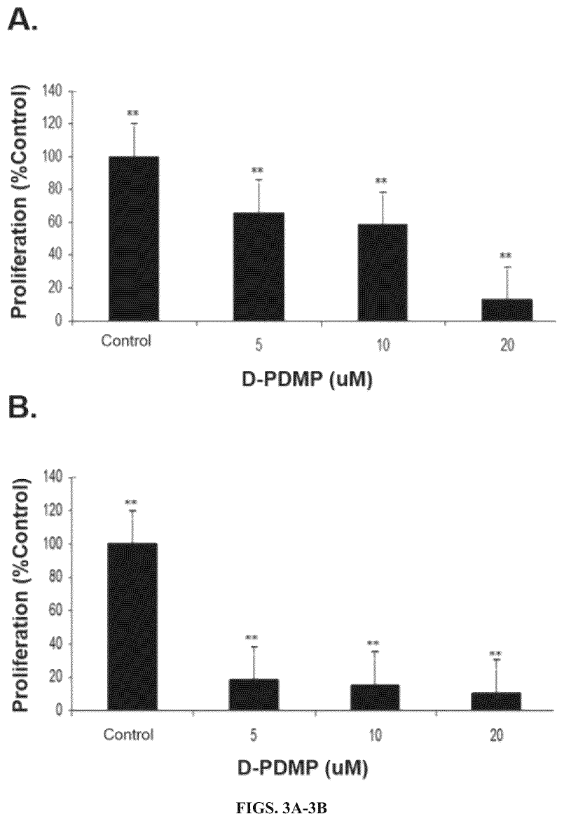

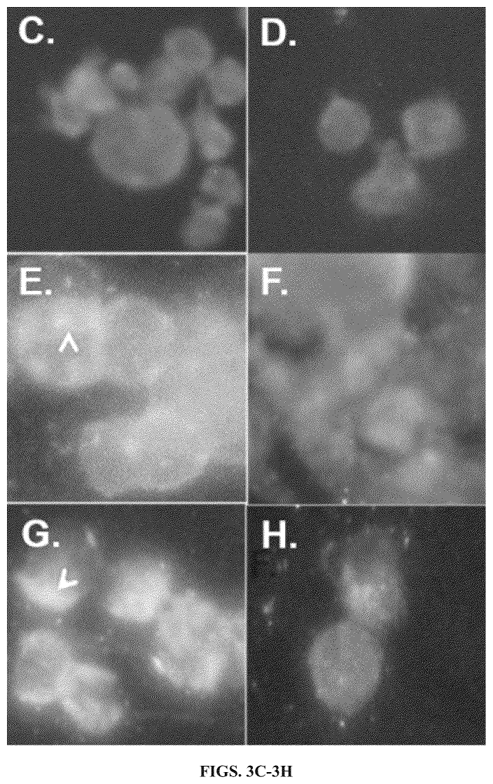

ELISA revealed a marked increase (approximately 6.5-fold) of β-1,4-GalT-V in CRC tissues, compared to visibly normal areas (i.e., “adjacent normals”) from the same tissue specimens (FIG. 1F). A 2.25-fold increase was observed in lactosylceramide synthase activity, in CRC samples, compared to those of normal colon epithelia (**P=0.0052, FIG. 1G). Quantitative RT-PCR further revealed elevated expression of several genes previously associated with CRC, e.g., adenomatous polyposis coli (APC) (26), N-myristoyltransferase 1 (NMT1), and tumor protein p53 (TP53), compared to normal samples (FIG. 1H). Moreover, β-1,4-GalT-V (B4GALT5) specifically showed increased expression, while β-1,4-galactosyltransferase, polypeptide 6 (B4GALT6) and UDP-glucose-ceramide β-1,4-glucosyltransferase (UGCG) did not (FIG. 1I). LC-MS revealed insignificant but modestly elevated levels of ceramide (Cer) (FIG. 2A), dihydroceramide (DHCer) (FIG. 2B), monoglycosylceramides (i.e. galactosylceramide (GalCer)) and glucosylceramide (GlcCer), FIG. 2C), dihydroGalCer/dihydroGlcCer (FIG. 2D), and dihydrolactosylceramide (DHLacCer) (FIG. 2F) in tumors, compared to normal colon specimens. However, among the GSLs investigated, only lactosylceramide (LacCer) levels were statistically and significantly increased in CRC tissues (FIG. 2E,*P=0.0112). CRC samples also showed elevated dihydrosphingomyelin (DHSM), compared to normal tissue (FIG. 2G,**P0.0059), and similar levels of sphingomyelin (FIG. 2G).

### Inhibition of GSL Synthesis Dose-Dependently Decreases Proliferation of Human Crc Cells.

HCT-116 cells were treated with D-threo-1-phenyl-2-decanoylamino-3-morpholino-1-propanol (D-PDMP, FIG. 3A), a potent inhibitor of UDP-glucose-cer: glucosyltransferase and LCS/3-1,4-galactosyltransferase (GalT-V) activity (27, 28, 29). It was found that in CRC cells, D-PDMP exerted a dose- and time-dependent (FIGS. 3A, 3B) decrease in proliferation, compared to control cells, with the maximal effective dose at 20 μM.

D-PDMP Treatment Reduces β-1,4-GalT-V Protein Expression and Activity (i.e. Sphingolipid Synthesis) in Human CRC Cells.

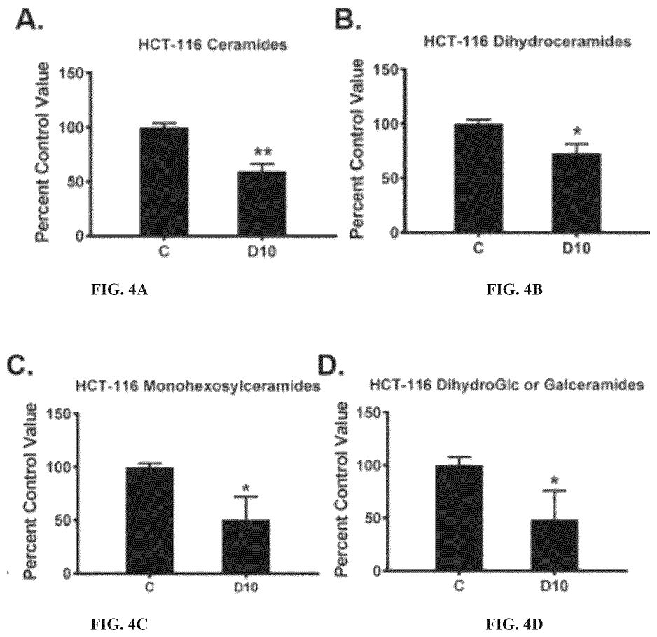

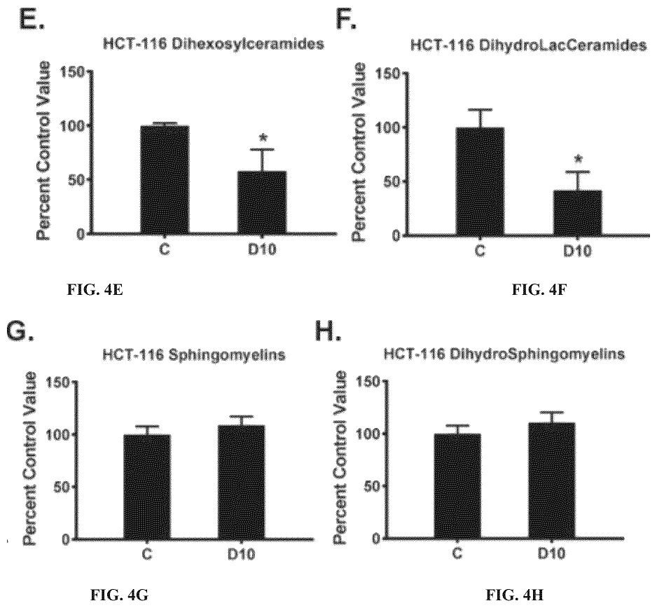

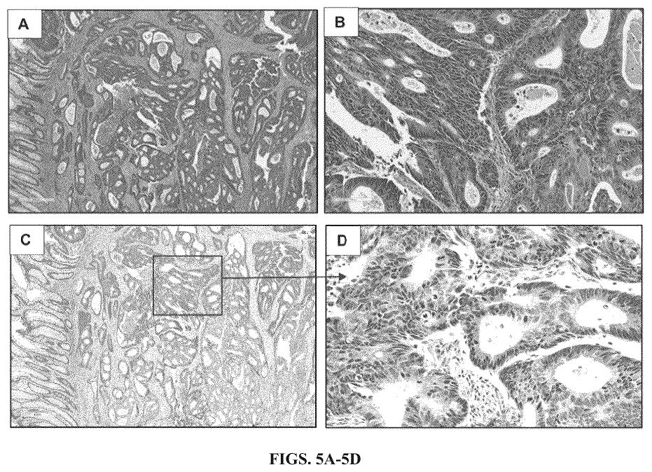

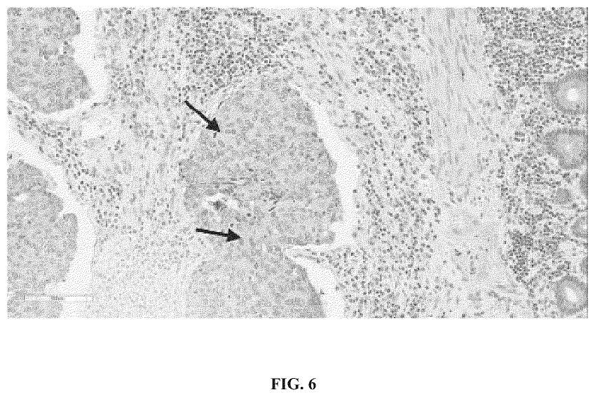

No difference was found in UGCG immunofluorescence in D-PDMP treated cells (FIG. 3D) compared to control (FIG. 3C) at 24 h. However, D-PDMP treatment for 24 (FIG. 3F) and 96 h (FIG. 3H) reduced GalT-V fluorescence compared to that of controls (FIGS. 3E and 3G, respectively). LC-MS analysis of GSL derived from HCT-116 cells treated with and without D-PDMP (10 μM) revealed reduced levels of Cer (FIG. 4A), DHCer (FIG. 4B), monohexosylceramides (FIG. 4C), dihydroGalCer/dihydroGlcCer (FIG. 4D), dihexosylceramide (FIG. 4E), DHLacCer (FIG. 4F) but not that of sphingomyelin (FIG. 4G) and DHSM (FIG. 4H), compared to control values.

### Discussion

Several major findings emerge from this study. First, it was observed that β-1,4-GalT-V expression, protein mass, and IHC staining were all markedly increased in human CRC tissue, as compared to visibly normal tissue. Second, the activity of β-1,4-GalT-V (i.e. synthesis of lactosylceramide), in human CRC tumors, was statistically and significantly higher, compared to control tissue. Third, inhibition of glycosphingolipid synthesis decreased immunostaining of β-1,4-GalT-V, consequently decreasing cell proliferation in cultured human CRC (HCT-116) cells. Fourth, enhanced dihydrosphingolipid metabolism (FIG. 2G) was noted in tumor tissues, leading to significantly increased levels of dihydrosphingomyelin, compared to visibly normal tissue.

Immunostaining using β-1,4-GalT-V antibody also allowed pathologists to clearly distinguish normal epithelial cells (FIG. 1A) from cancerous epithelial cells (FIGS. 1C-1E). After staining, the tissues were assessed for adequacy of tumor and 20 cases were selected where adequate tumor tissue was available for evaluation. Review of the immunostains for GalT-V revealed varying degrees of positivity in the tumor cells (Table 1): 1+(15%; 3/20), 2+(65%; 13/20), and 3+(20%; 4/20). Staining was mostly observed in the cytoplasm of tumors and very occasionally in the nuclei of tumor cells. Weak (1+) to moderate (2+) cytoplasmic staining was also observed in adjoining normal colonic mucosa in 79% cases (15/19), when available. Moreover, computerized (Asperion program) analysis of 20 such tissues provided a quantitative estimate of the immunostaining index, yielding a substantial H-score of 100 to 300 (from a scale of 0 to 300) (21). These observations provide evidence that β-1,4-GalT-V immunostaining could indeed serve as a novel biomarker for CRC progression. Strong β-1,4-GalT-V immunostaining of capillary endothelial cells was also observed consistent with the inventor's previous studies (30, 31). Within squamous epithelial cells, strong immunostaining was observed in the perinuclear area, suggesting the antigen to be enriched within the Golgi apparatus (data not shown).

β-1,4-GalT-V immunostaining of the cytoplasm, both in control and CRC tumor tissues, provides evidence that β-1,4-GalT-V must exist in a membrane-bound, as well as soluble form. Solubility would enable measurement of this antigen in various body fluids, via noninvasive or minimally invasive procedures. Since the brush border membrane in colonic epithelial cells also reacted positively to the antibody, this provides evidence that in colorectal tissue, GalT-V may be shed in exosomes.

Previous studies have shown that β-1,4-galactosyltransferases share a common stem region. Since in β-1,4-GalT-1 (lactosamine synthase), the stem region is short; most of the enzyme localizes to the Golgi, cytoplasm and relatively less with the plasma membrane (32). Additionally, it has been proposed that the number of hydroxylated amino acids, constituting the stem region, dictates the localization of a β-1,4-galactosyltransferase, although other factors may determine the localization of this protein. Further studies are warranted to examine whether alterations in the stem region allow for plasma membrane localization of β-1,4-GalT-V.

Moreover, enrichment of β-1,4-GalT-V as observed by IHC, was further substantiated by quantitative measurement of β-1,4-GalT-V protein mass. In CRC tissues, β-1,4-GalT-V mass was statistically and significantly increased, compared to control tissue (FIG. 1F). The activity of lactosylceramide synthase (and thus, lactosylceramide mass) was also significantly higher in CRC tissues, compared to controls (FIG. 1G). In contrast, differences in the levels of other sphingolipids, in tumor versus control tissue, were not statistically significant (FIGS. 2A-2G).

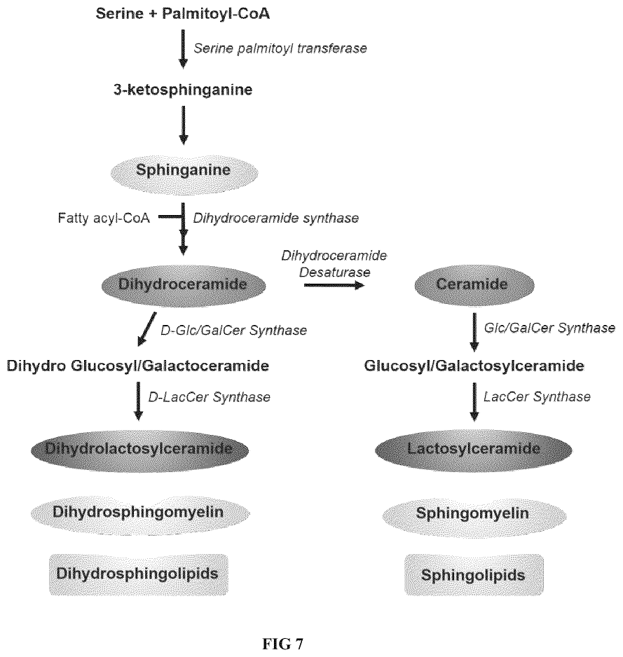

Another exciting finding of this study was that the dihydrosphingolipid pathway (FIG. 7) was significantly active in tumor tissue (FIG. 2G). For example, in tumor tissue, it was observed that the masses of dihydroceramide, dihydroGlcCer/dihydroGalCer, and dihydroLacCer were all modestly elevated (but not significant). However, the most significant difference between tumor tissue and visibly normal tissue was that the level of dihydrosphingomyelin was markedly increased in tumor tissue (FIG. 2G). Recent studies suggest that dihydrosphingolipids, e.g. dihydroceramide, play an important role in autophagy (33, 34). However, the role of dihydrosphingomyelin, in human CRC, needs to be explored.

The quantitative RT-PCR studies herein revealed increased B4GALT5 gene expression in tumor tissue, but relatively not for that of its isoform B4GALT6, or a homolog UGCG (FIG. 1I). These observations validated previously reported specific increases in β-1,4-GalT-V but not β-1,4-GalT-VI in CRC tumors, using serial analysis of gene expression technology (30). Thus, increased β-1,4-GalT-V gene and protein expression is specific for CRC tissue, and may well serve as a biomarker to diagnose this disease and measure drug response. Previously, several other genes have been suggested to serve as CRC biomarkers, including NMT1, APC, and TP53 (35, 36, 37). Accordingly, the expression of these genes were analyzed and upregulation was observed in all three, in CRC tumor versus normal tissue (FIG. 1H). Thus, β-1,4-GalT-V upregulation could feasibly be added to the three-biomarker panel, thus enhancing the predictive value of CRC diagnosis.

In HCT-116 cells, β-1,4-GalT-V immunostaining was also observed within the inner layer of the plasma membrane and cytosol. Moreover, immunoreactivity increased as the cells grew from 24 h to 96 h (FIGS. 3A-3H), correlating with increased expression of β-1,4-GalT-V. In contrast, inhibition of glycosphingolipid synthesis with D-PDMP, significantly diminished GalT-V immunoreactivity in HCT-116 cells (FIGS. 3A-3H), thus providing evidence that treatment may decrease β-1,4-GalT-V protein mass and dihydroglycosylceramides/LacCer levels in these cells, and consequently decrease cell proliferation (FIGS. 3A-3H).

Immunostaining of UGCG in HCT-116 cells was performed. It was similarly found that this antigen/enzyme localized to the perinuclear area and cytoplasm. However, treatment with D-PDMP did not decrease immunoreactivity to an anti-UGCG antibody. Thus, treatment might have reduced glucosylceramide levels in HCT-116 cells by inhibiting enzyme activity.

It was found that in addition to human CRC tumors, HCT-116 cells also had an active dihydrosphingolipid pathway. Treatment with D-PDMP decreased levels of all dihydrosphingolipid species in our study, except for those of dihydrosphingomyelin. Additional mechanistic studies are warranted to address this observation further. It was also found that the net result of D-PDMP treatment was a dose-dependent decrease in HCT-116 cell proliferation.

In summary, this study showed a specific increase in the gene expression, protein levels, and enzymatic activity of β-1,4-GalT-V, concurrent with increased lactosylceramide mass, in human CRC. These molecular and biochemical data were further substantiated by IHC and pathology studies. These findings demonstrate that levels of β-1,4-GalT-V and lactosylceramide in human liquid biopsy specimens could complement other currently used biomarkers (e.g., NMT1, APC and TP53), thus increasing the positive predictive value for CRC. Last, inhibition of glycosphingolipid synthesis may be a novel approach to treat human colorectal, and possibly other types of cancer.

### REFERENCES

(1) P. Favoriti, G. Carbone, M. Grego, et al., Worldwide burden of colorectal cancer: a review, Updates Surg. 68(1) (2016) 7-11. https://doi.org/10.1007/s13304-016-0359-y.
(2) H. Brenner, M. Kloor, C. P. Pox, Colorectal cancer, Lancet. 383(9927) (2014) 1490-1502. https://doi.org/10.1016/S0140-6736(13)61649-9.
(3) J. Ferlay, I. Soerjomataram, R. Dikshit, et al., Cancer incidence and mortality worldwide: sources, methods and major patterns in GLOBOCAN 2012, Int. J. Cancer. 136(5) (2015) E359-E386. https://doi.org/10.1002/ijc.29210.
(4) M. Arnold, M. S. Sierra, M. Laversanne, et al., Global patterns and trends in colorectal cancer incidence and mortality, Gut. 66(4) (2017) 683-691. https://doi.org/10.1136/gutjnl-2015-310912.
(5) H. Brenner, C. Stock, M. Hoffmeister, Colorectal cancer screening: the time to act is now, BMC Med. 13 (2015) 262. https://doi.org/10.1186/s12916-015-0498-x.
(6) T. Tanaka, M. Tanaka, T. Tanaka, et al., Biomarkers for colorectal cancer, Int. J. Mol. Sci. 11(9) (2010) 3209-3225. https://doi.org/10.3390/ijms11093209.
(7) S. Hundt, U. Haug, H. Brenner, Blood markers for early detection of colorectal cancer: a systematic review, Cancer Epidemiol. Biomarkers Prev. 16(10) (2007) 1935-1953. https://doi.org/10.1158/1055-9965.EPI-06-0994.
(8) K. Simon, V. Balchen, Colorectal cancer development and advances in screening. Clin. Interv. Aging. 11 (2016) 967-976. https://doi.org/10.2147/CIA.S109285.
(9) T. F. Imperiale, D. F. Ransohoff, S. H. Itzkowitz, et al., Multitarget stool DNA testing for colorectal-cancer screening, N. Engl. J. Med. 370 (2014) 1287-1297. https://doi.org/10.1056/NEJMoa1311194.
(10) S. I. Hakomori, W. T. Murakami, Glycolipids of hamster fibroblasts and derived malignant-transformed cell lines, Proc. Natl. Acad. Sci. USA. 59(1) (1968) 254-61.
(11) J. W. Dennis, S. Laferte, Oncodevelopmental expression of -GlcNAc beta 1-6Man alpha 1-6Man beta 1-branched asparagine-linked oligosaccharides in murine tissues and human breast carcinomas, Cancer Res. 49(4) (1989) 945-950.
(12) S. Hakomori, Tumor malignancy defined by aberrant glycosylation and sphingo(glycol)lipid metabolism, Cancer Res. 56(23) (1996) 5309-18.
(13) M. Garcia-Barros, N. Coant, J. P. Truman, et al., Sphingolipids in colon cancer, Biochim. Biophys. Acta. 1841(5) (2014) 773-782. https://doi.org/10.1016/j.bbalip.2013.09.007.
(14) Y. Y. Liu, R. A. Hill, Y. T. Li, Ceramide glycosylation catalyzed by glucosylceramide synthase and cancer drug resistance, Adv. Cancer Res. 117 (2013) 59-89. https://doi.org/10.1016/B978-0-12-394274-6.00003-0.
(15) β. Segui, N. Andrieu-Abadie, J. P. Jaffrezou, et al., Sphingolipids as modulators of cancer cell death: potential therapeutic targets, Biochim. Biophys. Acta. 1758(12) (2006) 2104-2120. https://doi.org/10.1016/j.bbamem.2006.05.024.
(16) J. Arango, M. Pierce, Comparison of N-acetylglucosaminyltransferase V activities in Rous sarcoma-transformed baby hamster kidney (RS-BHK) and BHK cells, J. Cell Biochem. 37(2) (1988) 225-31. https://doi.org/10.1002/jcb.240370209.
(17) K. Shirane, T. Sato, K. Segawa, et al., Involvement of beta-1,4-galactoslytransferase V in malignant transformation-associated changes in glycosylation, Biochem. Biophys. Res. Commun. 265(2) (1999) 434-8. https://doi.org/10.1006/bbrc.1999.1684.
(18) S. Xu, S. Zhang, C. Chen, et al., Over-expression of beta-1,4-galactosyltransferase V increases the growth of astrocytoma cell line, J. Exp. Clin. Cancer Res. 21(3) (2002) 409-414.
(19) Y. Wei, F. Zhou, Y. Ge, et al., β1,4-Galactosyltransferase V regulates self-renewal of glioma-initiating cell, Biochem. Biophys. Res. Commun. 396(3) (2010) 602-607.
   https://doi.org/10.1016/j.bbrc.2010.04.110.
(20) S. Chatterjee, Assay of lactosylceramide synthase and comments on its potential role in
   signal transduction, Methods Enzymol. 311 (2000) 73-81. https://doi.org/10.1016/S0076-6879(00)11068-7.
(21) Varghese, F., Bukhari, A. B., Melhotra, R., et al., 2014. IHC profiler: an open source plugin for the quantitative evaluation and automated scoring of immunohistochemistry images of
   human tissue samples. PLoS One 9(5), e96801.
   https://doi.org/10.1371/journal.pone.0096801. (22) D. Bedja, W. Yan, V. Lad, et al., Inhibition of glycosphingolipid synthesis reverses skin
   inflammation and hair loss in ApoE −/− mice fed western diet, Sci. Rep. 8 (2018) 11463. https://doi.org/10.1038/s41598-018-28663-9.
(23) Chatterjee, S., Alsaeedi, N., Hou, J., et al., 2013. Use of a glycolipid inhibitor to ameliorate renal cancer in a mouse model. PLoS One. 8(5), e63726. https://doi.org/10.1371/journal.pone.0063726.
(24) N. J. Haughey, R. G. Cutler, A. Tamara, et al., Perturbation of sphingolipid metabolism and ceramide production in HIV-dementia, Ann. Neurol. 55(2) (2004) 257-267.
   https://doi.org/10.1002/ana.10828.
(25) V. V. R. Bandaru, J. Troncoso, D. Wheeler, et al., ApoE4 disrupts sterol and sphingolipid
   metabolism in Alzheimer's but not normal brain, Neurobiol. Aging. 30(4) (2009) 591-599. https://doi.org/10.1016/j.neurobiolaging.2007.07.024.
(26) M. E l Zoghbi, L. C. Cummings, New era of colorectal cancer screening, World J.
Gastrointest. Endosc. 8(5) (2016) 252-258. https://doi.org/10.4253/wjge.v8.i5.252.
(27) S. Chatterjee, Sphingolipids in atherosclerosis and vascular biology, Arterioscler. Thromb. Vasc. Biol. 18(10) (1998) 1523-1533.
(28) S. Chatterjee, T. Cleveland, W. Y. Shi, et al., Studies of the action of ceramide-like substances (D- and L-PDMP) on sphingolipid glycosyltransferases and purified lactosylceramide synthase, Glycoconj. J. 13(3) (1996) 481-486.
   https://doi.org/10.1007/BF00731481.
(29) S. Chatterjee, A. K. Bhunia, A. Snowden, et al., Oxidized low density lipoproteins stimulate galactosyltransferase activity, ras activation, p44 mitogen activated protein kinase and c-fos expression in aortic smooth muscle cells, Glycobiology. 7(5) (1997) 703-710.
(30) A. Kolmakova, M. Rajesh, D. Zang, et al., VEGF recruits lactosylceramide to induce endothelial cell adhesion molecule expression and angiogenesis in vitro and in vivo, Glycoconj.
   J. 26(5) (2009) 547-558. https://doi.org/10.1007/s10719-008-9206-9.
(31) M. Rajesh, A. Kolmakova, S. Chatterjee, Novel role of lactosylceramide in vascular endothelial growth factor-mediated angiogenesis in human endothelial cells, Circ. Res. 97(8) (2005) 796-804. https://doi.org/10.1161/01.RES.0000185327.45463.A8.
(32) P. K. Qasba, β. Ramakrishnan, E. Boeggeman, Structure and function of beta-1,4-galactosyltransferase, Curr. Drug Targets. 9(4) (2008) 292-309.
   https://doi.org/10.2174/138945008783954943.
(33) W. Zheng, J. Kollmeyer, H. Symolon, et al., Ceramides and other bioactive sphingolipid
   backbones in health and disease: lipidomic analysis, metabolism and roles in membrane structure, dynamics, signaling and autophagy. Biochim. Biophys. Acta. 1758(12) (2006) 1864-
   84. https://doi.org/10.1016/j.bbamem.2006.08.009.
(34) M. M. Siddique, Y. Li, β. Chaurasia, et al., Dihydroceramides: from bit players to lead actors, J. Biol. Chem. 290(25) (2015) 15371-9. https://doi.org/10.1074/jbc.R115.653204.
(35) C. E. Ducker, J. J. Upson, K. J. French, et al., Two N-myristoyltransferase isozymes play unique roles in protein myristylation, proliferation, and apoptosis, Mol. Cancer Res. 3(8) (2005) 463-476. https://doi.org/10.1158/1541-7786.MCR-05-0037.
(36) T. Armaghany, J. D. Wilson, Q. Chu, et al., Genetic alterations in colorectal cancer, Gastrointest. Cancer Res. 5(1) (2012) 19-27.
(37) X. L. Li, J. Zhou, Z. R. Chen, et al., p53 mutations in colorectal cancer-molecular pathogenesis and pharmacological reactivation, World J. Gastroenterol. 21(1) (2015) 84-93. https://doi.org/10.3748/wjg.v21.il.84.
(38) N. S. Radin, J. A. Shayman, J. Inokuchi, Metabolic effects of inhibiting glucosylceramide
   synthesis with PDMP and other substances, Adv. Lipid. Res. 26 (1993) 183-213.
(39) J. Inokuchi, I. Mason, N. S. Radin, Antitumor activity via inhibition of glycosphingolipid
   biosynthesis. Cancer Lett. 38(1-2) (1987) 23-30. https://doi.org/10.1016/0304-3835(87)90196-0.
(40) J. Inokuchi, M. Jimbo, K. Momosaki, et al., Inhibition of experimental metastasis of murine Lewis lung carcinoma by an inhibitor of glucosylceramide synthase and its possible mechanism of action, Cancer Res. 50(20) (1990) 6731-6737.

### Example 2: Immunotherapy Using Lactosylceramide Synthase (β1-1,4 GalT-V) Antibody to Prevent Colorectal Cancer (CRC) In Vivo

It was hypothesized that β-1,4 GalT-V plays an important role in human CRC, and manipulating this enzyme may well mitigate tumor cell proliferation and metastasis—by way of inhibiting angiogenesis. To test this hypothesis, mouse monoclonal antibodies were prepared against β-1,4 GalT-V and determined its effect on proliferation and angiogenesis in human and mouse CRC cells, human umbilical vein endothelial cells and a mouse xenograft model of human CRC.

### Materials and Methods

Monoclonal antibody against a β-1,4 galactosyltransferase (GalT-V) peptide having the amino acid sequence (IGAQVYEQVLRSAYAKRNSSVND, SEQ ID NO: 5) was prepared and characterized in regard to its titer, and use in ELISA, western immunoblot assay and immunoprecipitation of mouse and human tissues. A human colorectal cancer cell line (HCT-116) was a gift from late, Dr. David Huso, Dept of Comparative Medicine, from the institution. A mouse colorectal cancer line MC-38 and thin tissue sections from mouse colorectal cancer were a gift from Dr. Cindy Sears, Dept of Oncology at the institution. Human umbilical vein endothelial cells were purchased from Clonetics and cultured in appropriate growth medium. Human microvascular endothelial cells were a gift from Ms. Stephanie Brindal in the department. Vascular endothelial growth factor was purchased from R and D Inc. Matrigel and all other reagents were from Sigma-Aldrich. Biopolymer-encapsulated D-PDMP was prepared as described (6).

### Cell Proliferation Assay

1×10⁴ HCT-116 cells and MC-38 cells were seeded in 96 well-sterile plastic trays and grown in 100 μL of Dulbecco's minimum essential medium containing 10% fetal calf serum in 5% CO₂-air humidified incubator at 37 for 24 hrs. Medium was replaced with fresh medium supplemented with 2% serum and 5 μCi/mL of (³H) thymidine. Increasing dilutions of β-1,4 GalT-V Antibody were added to the wells. Human IgG or mouse IgG served as a negative control and D-PDMP (10 μM) or 1 uM BPD served as a positive control in these experiments. Following incubation for 24 hrs, the experiment was terminated and the incorporation of (³H) thymidine into DNA was measured by scintillation spectrometry.

### Angiogenesis Assay

Angiogenesis assays were performed using a commercially available kit from Chemicon Inc. (7).

### Measurement of β-1, 4 GalT-V Activity

The activity of ββ-1, 4 GalT-V was measured in cells treated with and without β-1,4 GalT-V antibody as described previously (14).

### Measurement of Glycosphingolipids

The mass of GSL was measured by quantitative HPTLC as described (9).

### Prevention of Tumor Cell Growth.

Normal male and female mice (C57 BL-6) were purchased from The Jackson Laboratory and were fed regular mice chow. Semi-confluent culture of HCT-116 cells were harvested and the cell pellet resuspended in medium supplemented with Matrigel in a ratio of 70:30 (by volume). The dorsal hair of the mouse was removed with the use of Nair, a hair remover (Church and Dwight Co.), and the skin area devoid of hair was cleaned with an alcoholic swab. Next, 4×10⁶ HCT cell suspension was injected subcutaneously. One week later, either 100 μl of GalT-V Antibody or 100 μL of BPD (1 mg/kg. Body weight) was injected subcutaneously at the sight of tumor cell injection daily for 3 weeks. Since hair grew back in the shaven area, hair was removed using Nair to expose the skin area and mice were photographed and recorded.

### Immunohistochemistry

Thin tissue sections were cut from mice colorectal cancer tumor tissue and subject to immunohistochemical staining with β-1,4 GalT-V Antibody as described previously (8). Briefly, sections were de-paraffinized, hydrated, and incubated with a β-1,4 GalT-V antibody (1:600 dilution) for 30 min. A second antibody, anti-rabbit HRP was applied and a brown signal was developed using DAB chromogen detection (Leica Biosystems). Slides were next counterstained with hematoxylin, washed, dehydrated, cover slipped and photographed (8).

### Results

GalT-V Antibody is taken up by endothelial cells and HCT-116 cells in a time-dependent manner.

In human microvascular endothelial cells, fluorescent tagged GalT-V was taken up at 4° C. As the temperature was shifted to 37° C., a time-dependent increase was observed in the uptake of GalT-V Antibody into the cytoplasm and in the perinuclear area—representing the Golgi apparatus. Pre-incubation of cells with an excess amount of GalT-V peptide. Similarly, HCT-116 cells also took up fluorescent tagged β-1,4 GalT-V Antibody in a similar temperature and time dependent manner. These studies showed that β-1,4 GalT-V Antibody is taken up and internalized by human endothelial cells and HCT-116 cells in a time and temperature-dependent manner.

### GalT-V Antibody Inhibits Proliferation in Human and Mouse Colorectal Cancer Cells.

³H thymidine uptake studies revealed that in human colorectal cancer cells GalT-V antibody dose-dependently decreases cell proliferation and this inhibition was within the range of inhibition observed using a pharmacological inhibitor of β-1,4 GalT-V; D-PDMP.

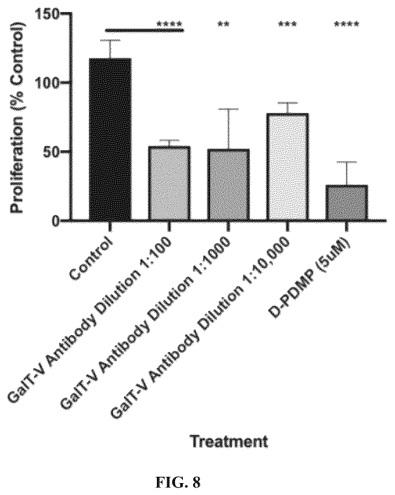

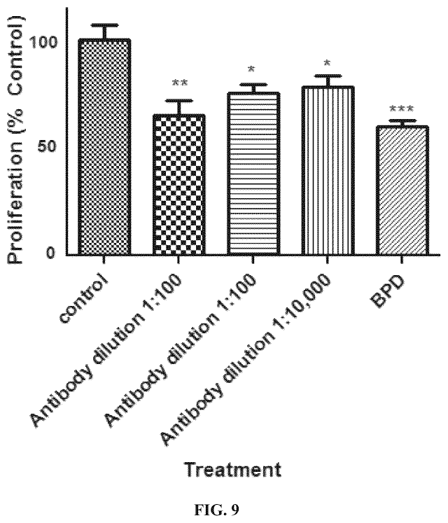

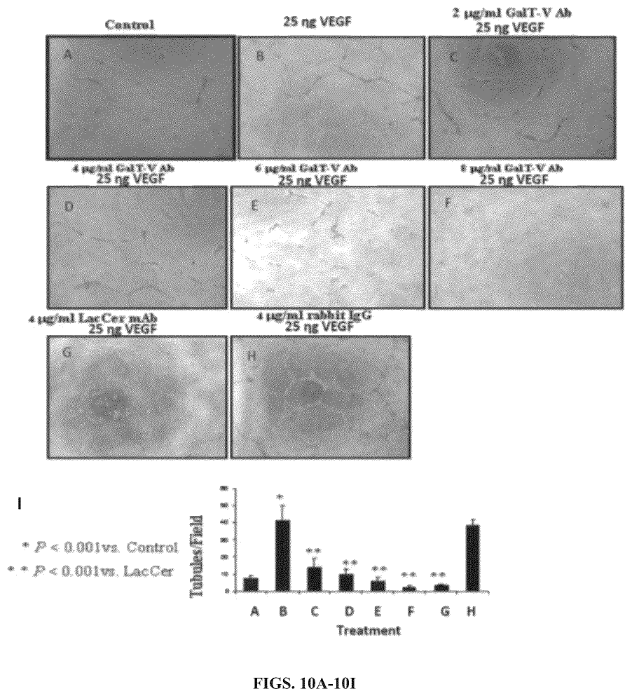

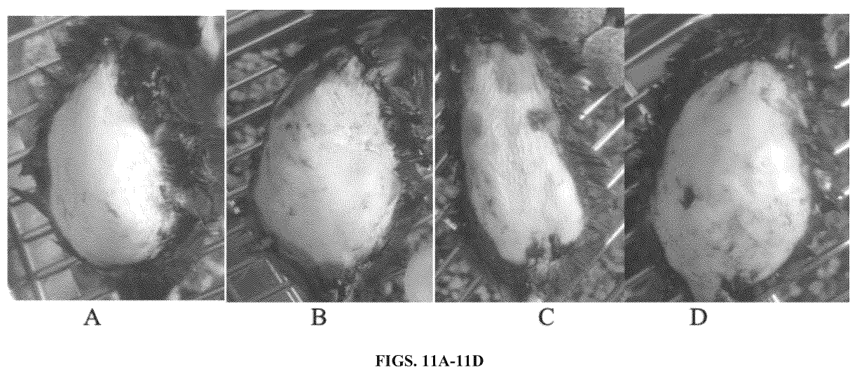

Similarly, β-1,4 GalT-V Antibody also dose-dependently inhibited the proliferation of mouse colorectal cells (FIG. 8); MC-38 (FIG. 9). Inhibition of cell proliferation was not observed when cells were incubated with mouse IgG.

### GalT-V Antibody Inhibits VEGF-Induced Angiogenesis in Human Umbilical Vein Endothelial Cells.

FIGS. 10A-10H show photographs of tube formation/angiogenesis in HUVEC's. FIG. 101 shows a corresponding quantification of angiogenesis. Incubation of HUVEC's with VEGF/FGF markedly increased tube formation/angiogenesis (FIG. 10B) compared with control (FIG. 10A). Treatment with β-1,4 GalT-V Antibody exerted a dose-dependent decrease in VEGF-induced angiogenesis in HUVEC's (FIGS. 10C10-F) but not by rabbit IgG (FIG. 10G).

### GalT-V Antibody Prevents Tumor Growth in Mice.

Seven days after mice were injected with HCT-16 cells, they were treated with and without ββ-1, 4 GalT-V Antibody daily for three weeks. In parallel another group—of mice were treated with BPD (5 mpk) daily for three weeks at the site of tumor cell injection. It was observed that treatment with either ββ-1, 4 GalT-V Antibody (FIGS. 11A, 11B) or BPD (FIGS. 11C, 11D) completely prevented tumor growth and progression.

### Discussion

This study lead to the following conclusions. (i). That treatment with β-1,4 Gal T-V Antibody dose-dependently decreased proliferation in cultured human colorectal cancer cells and mouse colorectal cancer cells. (ii). In human umbilical vein endothelial cells treatment with β-1,4 GalT-V Antibody dose-dependently decreased VEGF-induced angiogenesis. (iii). In a mouse xenograft model of colorectal cancer, treatment with β-1,4 GalT-V Antibody and BPD prevented tumor growth.

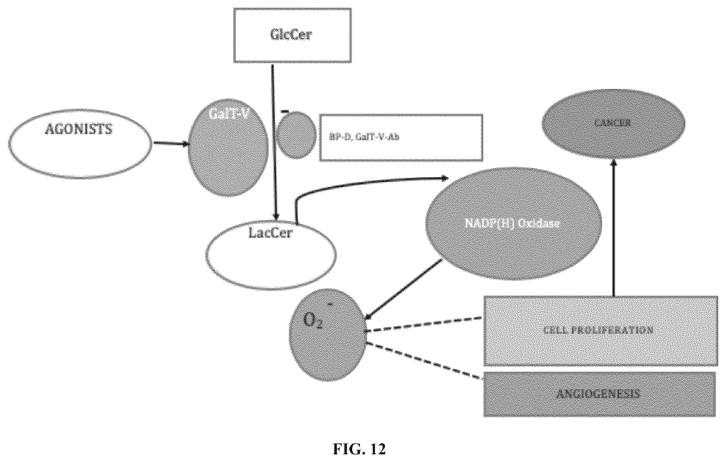

β-1,4 GalT-V is a member of a large family of galactosyltransferases whose function is to transfer galactose from UDP-galactose to glucosylceramide to form Lactosylceramide (1). It also transfers galactose to GlcNAc β-1,6 mannose group of the highly branched N glycans, which are characteristic of tumor cells (2, 3). Of these products, LC has been shown to serve as an independent mitogenic agent, angiogenesis agent as well implicated in cell migration, apoptosis and cell adhesion (4). Most importantly, LC serves as a surrogate to mediate the action of growth factors e.g. VEGF, FGF, PDGF, EGF and a pro-inflammatory cytokine TNF leading to the above phenotypes depending on the cell type (4). Importantly, these growth factor and TNFα-induced phenotypes can be mitigated by the use of pharmacological agents BPD and D-PDMP (6, 9) and GalT-V gene manipulation in vitro (7), and in vivo (15) (FIG. 12). This is the first report wherein ββ-1, 4 GalT-V immunotherapy is shown to be effective in reducing the growth and proliferation of colorectal cancer. The studies herein show that in human colorectal cancer tissue endothelial, cells ββ-1, 4 GalT-V mRNA levels are specifically increased and consequently the mass of ββ-1, 4 GalT-V protein is also increased in a cancer stage-dependent manner. While in normal colonic cells β-1, 4 GalT-V is found mostly in the cytoplasm/Golgi, in colorectal cancer it is present in addition in the brush border membrane in epithelial cells and to some extent in the cell membrane in human colorectal cancer cells (8). This allows the β-1, 4 GalT-V antibody to bind to the GalT-V protein. At 37° C. the fluorescent tagged β-1, 4 GalT-V antibody wase observed to bind to the antigen largely in the cytoplasm in normal human endothelial cells and HCT-116 cells (8). Since large doses of unlabeled antibody competitively inhibited the uptake of tagged antibody, suggest that β-1, 4 GalT-V antibody binding and uptake is specific in cultured colorectal cells (data not shown).

The inventor's previous studies has shown that LC generated due to growth factor-induced activation of β-1,4 GalT-V, activated NAD(P)H oxidase to generate reactive oxygen species which served as a signaling intermediate in a mitogen-activated protein kinase/c-fos pathway leading to cell proliferation (4). In this study it was observed that treatment with (β-1,4 GalT-V antibody reduces β-1,4 GalT-V enzyme activity and LC mass and therefore mitigated cell proliferation in HCT-116 cells.

Monoclonal antibodies are a specific type of antibodies/proteins made for therapeutic use. Such antibodies can be used in a targeted therapy to block an abnormal protein in a cancer cell. Monoclonal antibody can be used in immunotherapy as some of them attach specifically to a cancer cell expressing that protein. Thus by identifying cancer cells allows the immune system to attack and destroy it. Another type of antibodies affect cancer growth by releasing the brakes on the immune system so it can destroy the cancer cell. Studies show that programmed cell death (PD1)/programmed cell death ligand (PDL-1), CTLA-4 pathways are critical to the immune system's ability to control cancer growth. Such pathways are termed “immune check points”. Several types of cancer judicially use these pathways to escape the immune system. In contrast, immune check point inhibitors e.g., penbrolizumab (Keytruda) etc. are useful in identifying the blockade by the PD-L1 protein which acts like a protective shield in cancer cells. Recently, penbrolizumab has been approved by the FDA to treat tumors—metastatic cancers which cannot be treated by chemotherapy as well as Merkel skin cancer due to Merkel polyoma virus infection. Thus this check point inhibitor can target any tumor in the body and are therefore called tumor agnostic treatments. Nivolumab is a drug approved to treat CRC with M91-H or dMMR in patients after chemotherapy has failed. Interferons and interleukins have also been used to fight cancer and to develop immune system to generate cells which destroy cancer. The 2 year success rate for immunotherapy has been the highest (82%) in stage IV lymphomas and only 38% for patients with stage IV CRC. The β-1,4 GalT-V monoclonal antibody is of the IgG type and may well serve in targeted therapy to block the excess amounts of β-1,4 GalT-V protein found in human CRC tissue and decorating the inner aspect of cell membrane in endothelial cells and cytoplasm in cultured human CRC cells (8). Use of humanized β-1,4 GalT-V monoclonal antibody alone or in combination with BPD and/or other CRC drugs e.g. Nivolumab may well the be future direction of research to accelerate our therapeutic efforts to mitigate CRC. Since β-1,4 GalT-V protein is also over expressed in renal cancer (9) may find multiple uses of this immunotherapeutic approach.

### REFERENCES

1. Kolmakova A, Chatterjee S. Platelet derived growth factor recruits lactosylceramide to induce cell proliferation in UDPGal:GlcCer:β→4Galactosyltransferase (GalT-V) mutant Chinese hamster ovary cells. Glycoconj J 2005; 22: 401-7.
2. J. Arango, M. Pierce, Comparison of N-acetylglucosaminyltransferase V activities in Rous sarcoma-transformed baby hamster kidney (RS-BHK) and BHK cells, J. Cell. Biochem., 37 (2) (1988), pp. 225-231.
3. K. Shirane, T. Sato, K. Segawa, et a!. Involvement of beta-1,4-galactoslytransferase V in malignant transformation-associated changes in glycosylation, Biochem. Biophys. Res. Commun., 265 (2) (1999), pp. 434-438.
4. Chatterjee, S. and Pandey, A. The Yin and Yang of lactosylceramide metabolism: implications in cell function. Biochem.Biophys Acta. 1780:370-382, 2007.
5. Chatterjee, S. Kolmakova, A and Mohanraj, R. Regulation of lactosylceramide synthase; implications as a drug target. Curr. Drug Targets 9: 272-281, 2008.
6. D. Bedja, W. Yan, V. Lad, et al. Inhibition of glycosphingolipid synthesis reverses skin inflammation and hair loss in ApoE −/− mice fed western diet, Sci. Rep., 8 (2018), p. 11463.
7. M. Rajesh, A. Kolmakova, S. Chatterjee. Novel role of lactosylceramide in vascular endothelial growth factor-mediated angiogenesis in human endothelial cells, Circ. Res., 97 (8) (2005), pp. 796804.
8. S. Chatterjee, et al. Lactosylceramide synthase B-1,4 GalT-V: A novel target for the diagnosis and therapy of human colorectal cancer. Bio Chem. Biophys. Res. Comm. 2019; 508, 380-400.
9. S. Chatterjee, N. Alsaeedi, J. Hou, et al., Use of a glycolipid inhibitor to ameliorate renal cancer in a mouse model, PloS One, 8 (5) (2013), Article e63726.
10. P. Favoriti, G. Carbone, M. Grego, et al. Worldwide burden of colorectal cancer: a review, Updates Surg, 68 (1) (2016), pp. 7-11.
11. S. I. Hakomori, W. T. Murakami, Glycolipids of hamster fibroblasts and derived malignant-transformed cell lines, Proc. Natl. Acad. Sci. U.S.A., 59 (1) (1968), pp. 254-261.
12. J. W. Dennis, S. Laferte, Oncodevelopmental expression of -GlcNAc beta 1-6Man alpha 1-6Man beta 1-branched asparagine-linked oligosaccharides in murine tissues and human breast carcinomas, Cancer Res., 49 (4) (1989), pp. 945-950.
13. S. Hakomori, Tumor malignancy defined by aberrant glycosylation and sphingo(glycol)lipid metabolism, Cancer Res., 56 (23) (1996), pp. 5309-5318.
14. Y. Y. Liu, R. A. Hill, Y. T. Li, Ceramide glycosylation catalyzed by glucosylceramide synthase and cancer drug resistance, Adv. Cancer Res., 117 (2013), pp. 59-89.
15. Y. Wei, F. Zhou, Y. Ge, et al., β1,4-Galactosyltransferase V regulates self-renewal of glioma-initiating cell, Biochem. Biophys. Res. Commun., 396 (3) (2010), pp. 602-607.

### Example 3: β-1,4 GalT-V Monoclonal Antibody to Mitigate Atherosclerosis and Reduction in Body Weight in Type H Diabetic Mice(db/db)

Male Type II diabetic mice (db/db) aged 11 weeks were purchased from the Jackson Laboratory. They we raised on a normal mice chow and water. At the age of 30 weeks, mice were divided into two groups. The first group of mice (Placebo) were given saline (100 μL) daily by intra peritoneal injection for 6 weeks. The second group of mice were treated with GalT-V antibody (1 mg/kg body weight) for the same duration. At the end of 36 weeks of age mice, were weighed and various tissues were collected and frozen away until further analysis. Next, to extract total lipids, about 10 mg of liver tissue was excised, (internal standards of C12 ceramide and C12 sphingomylein were added to check recovery) and homogenized in acetonitrile and centrifuged at 1000 rpm for min. The clear supernatant was saved and the pellets was subject to repeated extraction. The pooled supernatant were dried in N2 and reconstituted in chloroform-methanol (2:lv/v). A suitable aliquot of the lipid extract was loaded onto a high performance thin layer chromatography plate. Also a misinterpretation of standard neutral lipids consisting of cholesterol ester, triglyceride and cholesterol were also loaded to calibrate the plate. The plate was developed using Heptane:ethyl-ether and acetic acid(65:16: lv/v) as solvent. The lipids were identified by exposing the plate with iodine vapors and photographed. Quantification of the mass of lipids was carried out by densitometric analysis using standard curves for individual lipids and using a two-tailed parametric t-test.

The results demonstrated that diabetic mice treated with GalT-V monoclonal AB had significantly reduced level of cholesterol as compared to placebo group of mice. Treatment also markedly reduced the level of triglycerides (neutral fat) as compared to placebo group of mice. In addition treatment reduced mice body weight by ˜20%.

### Example 4

We have shown that in cultured human CRC cells (HCT-116), treatment with GalT-V AB dose-dependently mitigated cell proliferation and angiogenesis [1]. Additionally, we have shown the enrichment of CF-750 tagged GalT-V-Ab in a xenograft tumor in NOD-SCID mice model of CRC.

We further wish to determine whether treatment with GalT-V-Ab affect tumor growth and metastasis in mouse orthotopic tumors in mice rectum.

### Methods

Rectal cancer was induced in NOD-SCID/immunocompromised mice by injecting live human colorectal cancer cells (HCT-116) (1×10⁶ cells in 50 μL in McCoy's medium). The treatment group of mice received 50 μL PBS (Placebo), 20 μg/kg GalT-V-Ab, and 200 μg/kg GalT-V-Ab by IV injection in the tail vein.

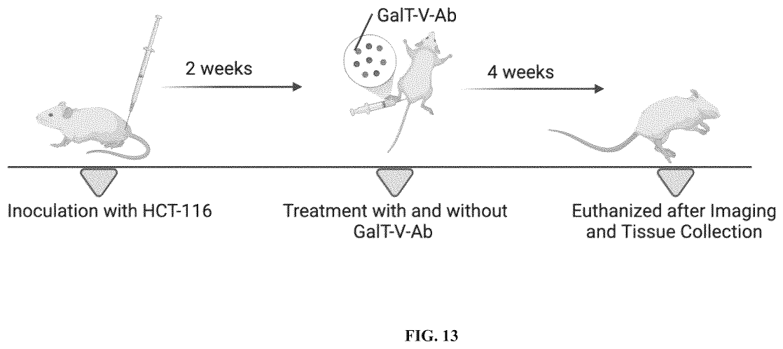

FIG. 13 summarizes a scheme followed to study the effects of treatment with GalT-V antibody in a mouse model of rectal cancer. Briefly, HCT-116 cells were grown in tissue culture to ˜75% confluence. Cells were harvested using trypsin, centrifuged and cell count was performed. 1×10⁶ cells suspended in serum-free McCoy's medium were injected into the rectum in male NOD SCID mice (˜10-week-old). Two weeks later, when rectal tumors were visible and quantified, treatment began. Mice were divided into three groups: A. Placebo, B. 20 ug GalT-V-Ab/kg body weight, and C. 200 ug GalT-V-Ab/kg body weight. Treatments were given by IV into the tail vein on alternate days and body weight and tumor size recorded. Four weeks after treatment, mice were divided into two groups: 1. A few mice were used in imaging the tumor and 2. rest of the mice were euthanized, and blood was drawn to prepare plasma and various tissues harvested. One half of the tumor tissue was saved in a formalin solution and used in lipid analysis as well as immunohistochemistry studies. The other half of the tumor tissue was flash frozen and used in molecular studies.

### Results

Treatment with GalT-AB antibody dose-dependently reduces rectal tumor volume in an orthotopic model of CRC in NOD-SCID mice.

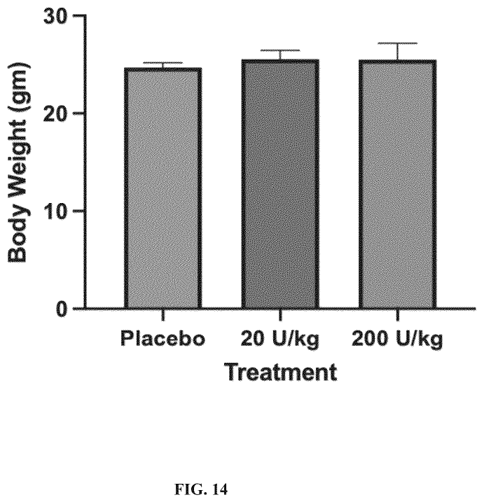

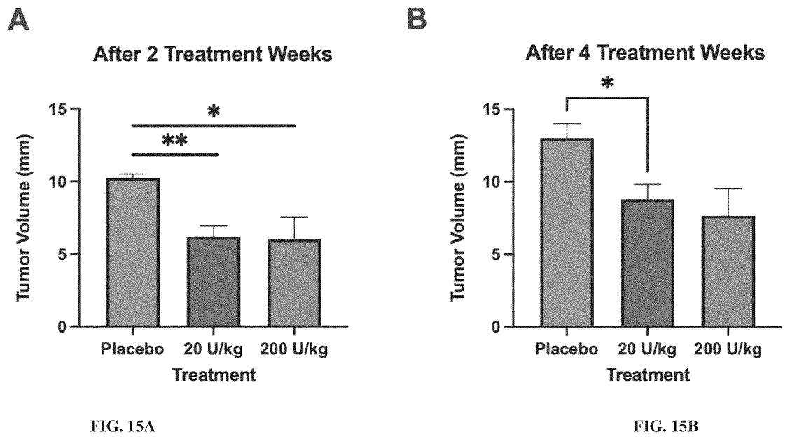

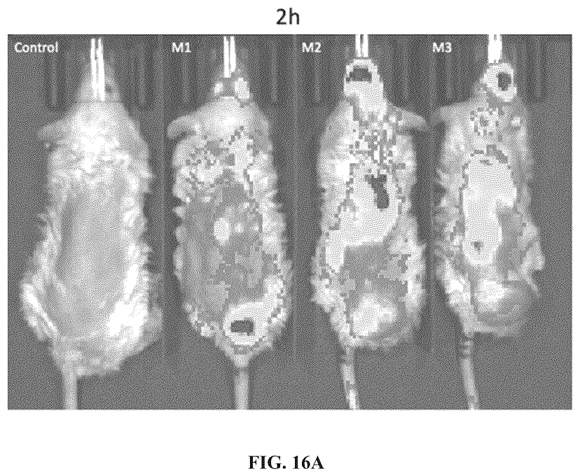

The body weight of mice did not change irrespective of treatment over a period of 4 weeks (FIG. 14). Treatment with GalT-V-Ab had a dose and time-dependent reduction in tumor volume. For example, two weeks after treatment we noted that tumor volume was reduced ˜38-41% in mice receiving 20 μg/kg and 200 μg/kg of the antibody (FIG. 15A). After four weeks of treatment tumor volume in mice treated with 20 and 200 μg/kg antibody were 32% and 41% lower respectively, compared to placebo mice bearing rectal tumor (FIG. 15B).

### Molecular Imaging of Reduction in Tumor Volume:

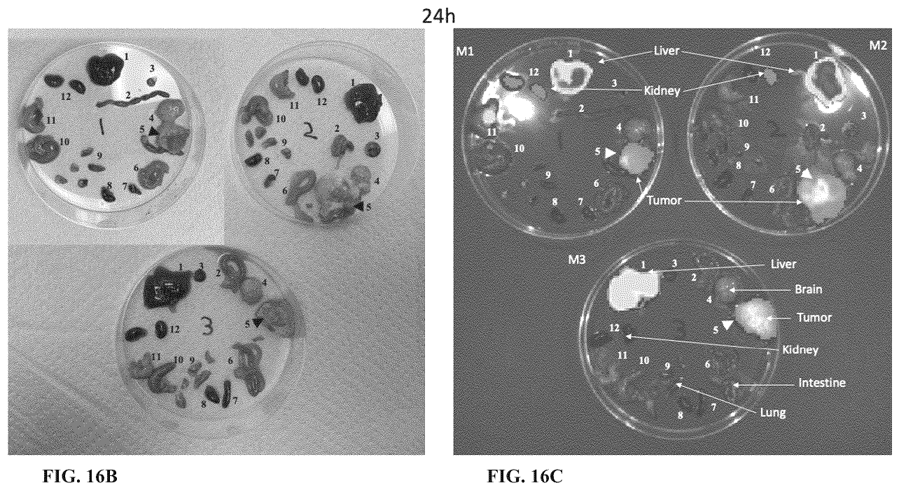

Four weeks after treatment, mice were injected with 50 μL of CF-750 tagged carcinoembryonic antigen (CEA), an established tumor biomarker. Two hours later whole mice were imaged as shown in FIG. 16A.

In FIG. 16, optical imaging of mice bearing HCT-116 rectal orthotopic tumor is hown: liver (1), large intestine (2), blood (3), brain (4), tumor (5) indicated by the black arrow, small intestine (6), spleen (7), heart (8), lungs (9), cecum (10), stomach (11), kidney (12) shown in a clockwise order.

HCT-116 human CRC tumor cells (1×10⁶) were implanted in the rectum of NOD-SCID male mice (10 weeks) old. Two weeks later treatment was begun with IV injections on alternate days for 4 weeks. The mice were imaged (FIG. 16A) using a Forager imaging machine 2 hours after the delivery of CF-750-antibody against carcinoembryonic antigen (CEA): control, mice with no CEA-Ab, M1. Mice treated with 200 ug GalT-V Ab/Kg body weight, M2, mice given placebo and M3. mice treated with 20 ug GalT-V Ab/Kg body weight. Twenty-four hours later, mice tissues were harvested, placed on a petri dish and photographed (FIG. 16B) and finally imaged (FIG. 16C).

The imaging studies involved preparing CF-750 tagged antibody against human CEA and delivering it by an IV injection in the tail vein. As shown in FIG. 16A whole mouse body imaging given placebo (M2 in FIG. 16A) revealed extensive localization/concentration of the tag into the rectum(green), liver(blue) and head(blue). Relatively less tag was found associated with these tissues in mice given 20 μg of GalT-V Ab (M3 in FIG. 16A). However, upon treatment with 200 μg of GalT-V-Ab most of the tag was concentrated in the rectum tumor(blue) (M1 in FIG. 16A).

FIG. 16B shows photographs of individual mice tissues before imaging and after imaging 24 hours after the injection of CF-750-CEA-Ab (FIG. 16C). Placebo tumor (FIG. 16C, M2) took up the highest level of the tag(red). Intensity of staining was highest in placebo liver (M2 in FIG. 16C) relative to GalT-V-Ab treated group. Some tag was found in lungs, kidney and blood(red). We also note that the intensity of staining decreased dose-dependently into the tumor tissue (red) upon treatment with 20 (M3 in FIG. FIG. 4C) and 200 ug of GalT-V-Ab/kg (M1 in FIG. 16C). Since we used intact CEA-Ab (IgGI) it entered the liver via portal circulation as well the kidneys and lungs. And this observation agrees with previous reports. In the future we plan to use only the CF-750-Fab fragment of CEA-Ab as it is taken up by tumor tissue vs liver at a ratio of >10:1.

Treatment with GalT-V Antibody and the Expression of Tumor Marker Genes:

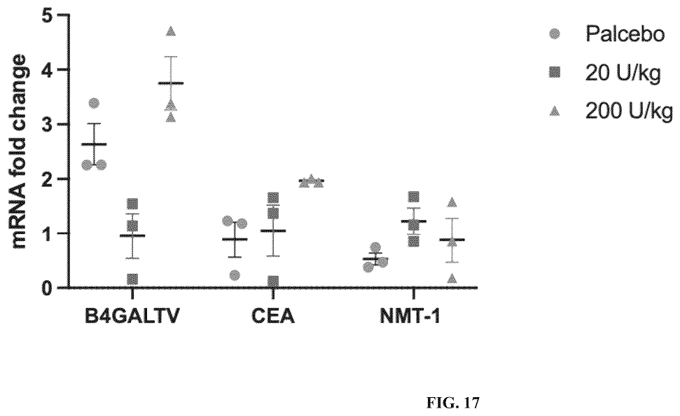

Quantitative analysis of gene expression through reverse-transcription polymerase chain reaction (RT-PCR) shows that mRNA level of CEA and NMT-1 are similar in the placebo and treatment groups (ns). However, the expression of B4GALT-V is decreased in the 20 U/kg group compared to the control and was not significant in the 200 U/kg group. See FIG. 17.

Treatment with GalT-V Antibody Reduced the Blood Level of GalT-V and Tumor Level of Lactosylceramide.

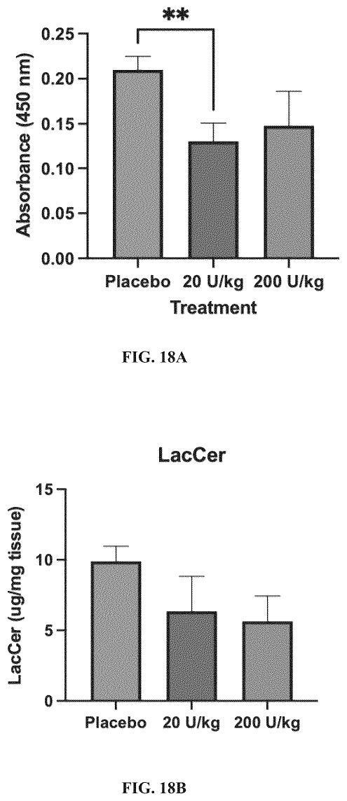

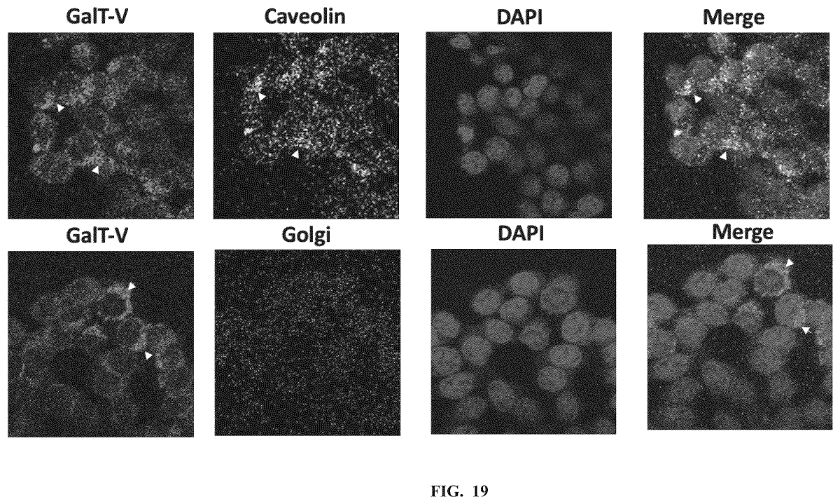

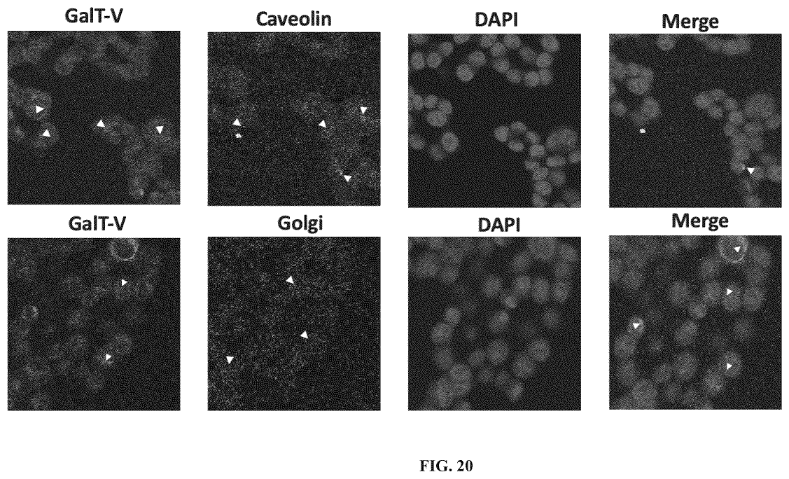

Our previous study in human CRC tissue revealed that the mass of GalT-V and the level of LacCer both were increased compared to visibly normal tissue from the same CRC patient. We also showed that treatment with D-PDMP; an inhibitor of GalT-V reduced the level of GalT-V and mass of LacCer in HCT-116 cells, and reduced cell proliferation. And in human endothelial cells, treatment with D-PDMP and with GalT-V-Ab reduced angiogenesis. Last, in a mouse model of renal cancer treatment with D-PDMP markedly reduced tumor volume and GalT-V mass [2]. Hence, we measured the mass of GalT-V in plasma and the level of LacCer in tumor tissue. As shown in FIG. 18A, treatment with GalT-V-Ab significantly reduced the level of GalT-V in plasma. And there was a trend of decreased level of LacCer in tumor tissue in the GalT-V treated group of mice (FIG. 18B).

### SUMMARY

1. In an orthotopic mice model of rectal cancer wherein live HCT-116 cells were inoculated there was a marked increase in tumor volume over a period of 6 weeks.

2. Treatment with GalT-V-Ab dose and time-dependently reduced tumor volume in the order of 32%-38% 2-4 weeks in mice given 20 ug/kg of GalT-V Ab.

3. Treatment with a higher dose of GalT-V-Ab reduced tumor volume by 41% compared to placebo tumor volume.

4. The Biochemical and molecular basis of this observation was explained as we observed that treatment targeted the antigen GalT-V by reducing its mass in blood as well it's product LacCer in tumor tissue by way of reducing GalT-V gene expression at least when mice were treated with 20 ugGalT-V Ab/kg.

5. Imaging studies recapitulated the observations above. Additionally, imaging studies revealed that in placebo mice, 6 weeks after inoculation, tumor metastasis occurred to other tissues e.g. liver, kidney and lungs—the major tissues in blood circulation. This was significantly mitigated when mice were treated with the low or high dose of GalT-V-Ab and markedly diminished in mice treated with the higher dose of GalT-V-Ab.

Immunotherapy using GalT-V-antibody is an effective therapy to prevent or inhibit the growth and metastasis of rectal tumor.

### Example 5

Localization of GalT-V and co-localization with cell-organelle specific biomarkers of cell surface protein and Golgi apparatus in human colorectal cancer cells was determined.

In vitro confocal fluorescent images of HCT-116 cells were taken (see FIG. 19). Cell surface localization of labeled mouse monoclonal antibody (mAb) raised against human GalT-V (red, rhodamine, Sigma Aldrich) and monoclonal anti-caveolin-1 antibody produced in mouse (green, Alexa Fluor 488 NHS ester, ThermoFisher Scientific) at 4° C. after 1 hour of incubation was observed. As well as co-localization of GalT-V and caveolin when the images were merged (yellow, note white arrows). Co-localization with monoclonal anti-Golgi 58 k protein (green, Alexa Fluor 488 NHS ester, ThermoFisher Scientific) at 4° C. was not observed when corresponding images were merged.

Mouse monoclonal antibody (mAb) against human GalT-V were tagged with rhodamine. Caveolin-1 Ab and Golgi Ab also were tagged with indocyanine green. Then, these antibodies were used to determine the localization of GalT-V using confocal microscopy (followed protocol from DOI: 10.1021/acs.molpharmaceut.0c00457). We also stained the nucleus with DAPI stain (ThermoFisher Scientific) which binds to DNA (blue). As shown in FIG. 19 (top panel) at 4° C. GalT-V (red stain) and caveolin (green stain) antibodies strongly bound to the cell surface on a human colorectal cancer cell line, HCT-116. When these figures were merged (top panel to the extreme right) we observed that the GalT-V and caveolin immunostaining overlap exhibiting a change in color to yellow (indicated by white arrows). When cells were incubated with methyl-cyclodextrin—a blocker of internalization, most of the GalT-V immunostaining was associated with the cell surface. In contrast, very little if any immunostaining were observed when cells were treated with an antibody against the Golgi apparatus (FIG. 19 bottom panel). Confocal microscopy images were taken on Zeiss LSM700 using 20× magnification and processed on Image-J.

### Example 6

In vitro confocal fluorescent images of HCT-116 cells with fluorescently tagged antibodies against GalT-V (red), caveolin (green) and Golgi (green). See FIG. 20. Cellular internalization of GalT-V antibody (red) and caveolin (green) antibody after incubation at 37° C. for 2 hours was observed. Limited co-localization of GalT-V and caveolin antibody was observed when the images were merged (yellow, see white arrow).

Next, cells were warmed to 37° C. for 2 hours, and then confocal microscopy was conducted. As shown in FIG. 20 top panel, GalT-V Ab (red stain) had internalized the cells and was associated with the cytoplasm. Since caveolin, contained within coated pits, is known to be internalized and return the cell surface, we observed cytoplasm and cell-surface immunostaining (green stain). Faint immunostaining with the Golgi antibody (green stain) was observed in the bottom panel.

### Example 7: Binding and Internalization of [⁸⁹Zr] GalT-V Antibody in Human Coronary Arterial Endothelial Cells (HAEC) and Human Colorectal Cancer Cells (HCT-116)

Cell cultures were incubated with increasing concentrations of [⁸⁹Zr] GalT-V antibody, washed and radioactivity was measured in an automated gamma counter (1282 Compu-gamma CS, Pharmacia/LKB Nuclear, Inc.). Note that HCT-116 cells bind significantly more amount of GalT-V antibody relative to HCAEC cells (n=3, p****(5 ug/mL)=0.000053, p***(10 ug/mL)=0.000805).

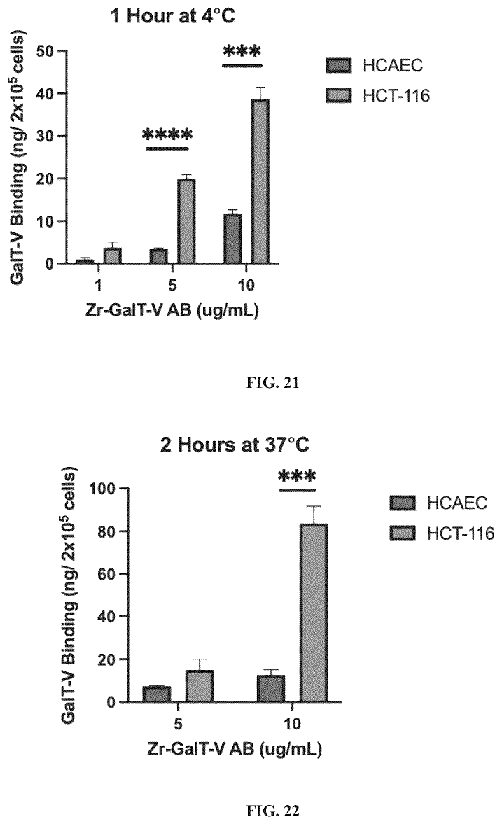

FIG. 21 shows [⁸⁹Zr] GalT-V antibody binding in human coronary arterial endothelial cells (HCAEC) and human colorectal cancer cells (HCT-116) after 1 hour incubation at 4° C.

### Example 8: [⁸⁹Zr] GalT-V Antibody Binding in Human Coronary Arterial Endothelial Cells (HCAEC) and Human Colorectal Cancer Cells (HCT-116)

Cell cultures were incubated with increasing concentrations of [⁸⁹Zr] GalT-V antibody, washed and radioactivity was measured in a gamma counter. Note that HCT-116 cells bind and internalize significantly more amount of GalT-V antibody relative to HCAEC cells (n=3, p***(10 ug/mL)=0.000124). Results are set forth inn FIG. 22, which shows [⁸⁹Zr] GalT-V antibody binding in human coronary arterial endothelial cells (HCAEC) and human colorectal cancer cells (HCT-116) after 2-hour incubation at 37° C.

### Example 9

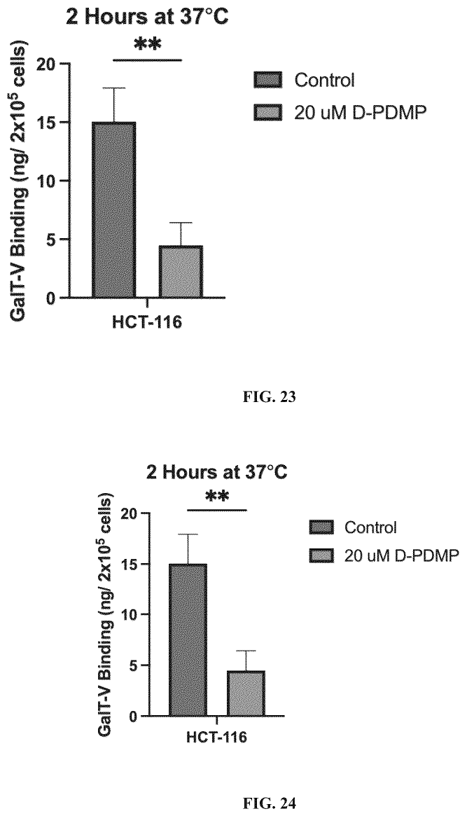

Cells were pre-incubated with and without an inhibitor of GalT-V, D-PDMP (20 uM), followed by the measurement of binding and internalization of [⁸⁹Zr] GalT-V antibody. As D-PDMP reduces the mass of GalT-V, it also reduced the binding and internalization of [⁸⁹Zr] GalT-V antibody (n=3, p**=0.0079). Results are depicted in FIG. 23 which shows D-PDMP inhibits zirconium tagged GalT-V antibody binding in human colorectal cancer cells (HCT-116).

### Example 10

Cells were incubated in the presence of excess unlabeled (cold, 50 ug/mL) GalT-V antibody as well as [⁸⁹Zr] GalT-V Ab. Note that the presence of unlabeled antibody markedly diminished the binding and internalization of [⁸⁹Zr] in HCT-116 cells treated. (n=3, p****=<0.0001). Results are shown in FIG. 24 which depicts specificity of binding and internalization of [⁸⁹Zr] GalT-V antibody in human colorectal cancer cells (HCT-116).

### Example 11

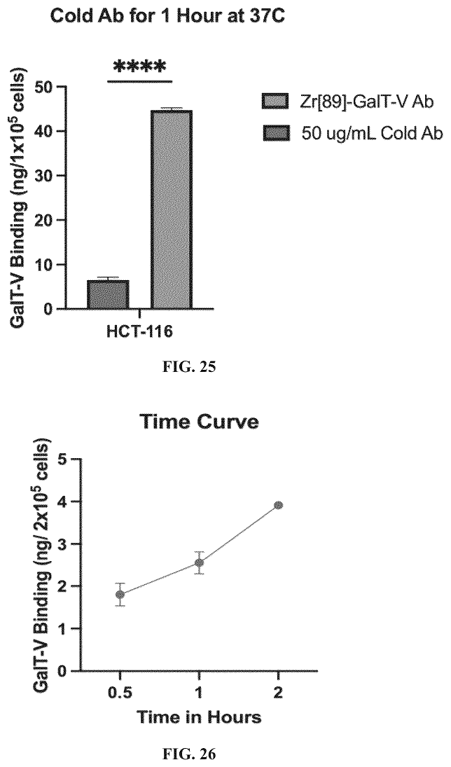

The GalT-V antibody was radiolabelled with [⁸⁹Zr] as it is a strong gamma-emitter and is useful in in vitro studies as well as in vivo studies in mice. As shown in FIG. 21, [⁸⁹Zr] GalT-V Ab binding to HCAEC cells (blue) and HCT-116 cells (red) measured at 4° C. was concentration dependent. Moreover, GalT-V Ab binding to HCT-116 cells was statistically significantly higher compared to HCAEC cells both at 4° C. (FIG. 21) and at 37° C. (FIG. 22). D-PDMP, which is known to inhibit GalT-V activity and mass in HCT-116 cells, also bound less GalT-V Ab compared to untreated cells (FIG. 23). The specificity of [⁸⁹Zr] GalT-V Ab binding was assessed by incubating HCT-cells with an excess of GalT-V antibody, which can compete with the radiolabeled Ab. As shown in FIG. 24, HCT-116 cells incubated with 50 μg/mL cold, unlabeled GalT-V markedly diminished (blue bar) binding compared to cells incubated with hot, radioactive GalT-V Ab (red bar). A study of the time course of [⁸⁹Zr] GalT-V Ab binding to HCT-116 cells (FIG. 25) showed an almost linear binding from 30 minutes to 2 hours in HCT-116 cells.

### Example 12: In Vivo Xenogen Fluorescence Images of Human CRC Tumor Bearing Mice

In vivo xenogen fluorescence images of human CRC tumor bearing mice were taken. 5 hours post injection of CF-750-GalT-V antibody depicts subcutaneous/xenotropic CRC tumor indicated with a black arrow as shown in FIG. 26.

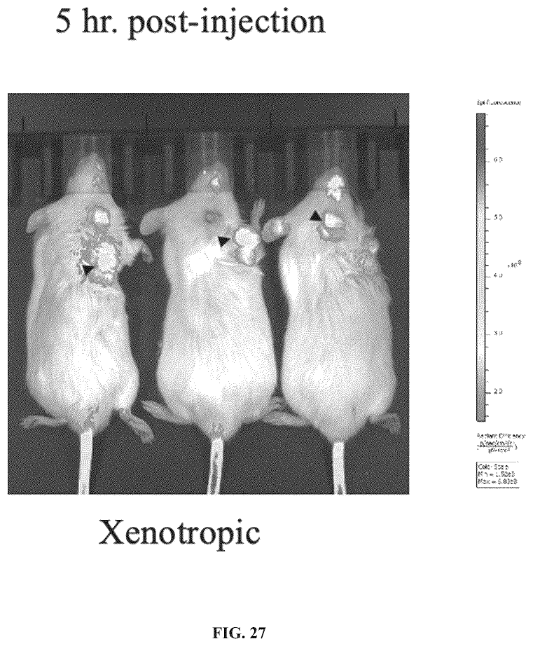

48 hours post injection of fluorescence-tagged GalT-V antibody depicts xenotropic CRC tumor indicated with a black arrow in FIG. 27. Note that the intensity of CF-750-GalT-V antibody has markedly increased from 5 hours post injection to 48 hours post injection (blue spots) in mice bearing xenotropic CRC tumors.

As shown in FIG. 26, we were able to image the xenotropic CRC tumors in mice within 5 hours after injection of the CF-750-GalT-V antibody (indicated with a black arrow). The bar on the right refers to the intensity of fluorescence. Deep blue and red color represent very high and very low GalT-V antibody fluorescence intensity, respectively. As the fur in these mice were auto fluorescent, we were unable to image the other internal organs non-invasively after anesthesia e.g., liver, spleen etc. in intact animals. We observed a time-dependent enrichment of fluorescence in the xenotropic tumors as noted by the change in color from green to blue (compare FIG. 26 at 5 hours and FIG. 27 at 48 hours post injection with the fluorescently tagged GalT-V antibody).

### Example 13: Tissue Distribution of CF-750 Fluorescent GalT-V Antibody in Mice Bearing Xenotropic Tumor

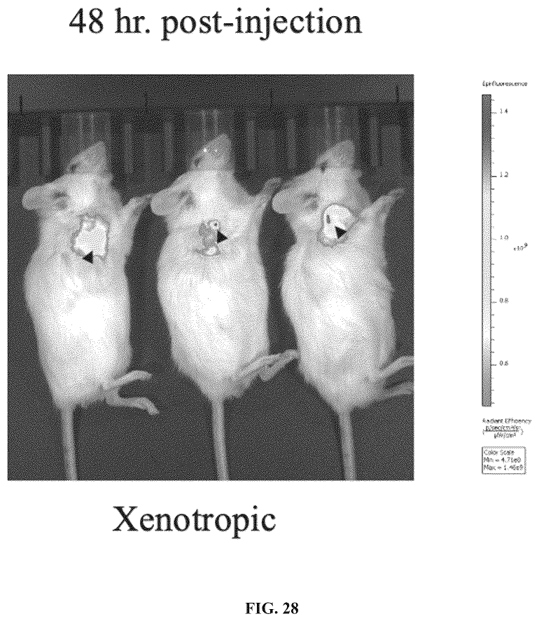

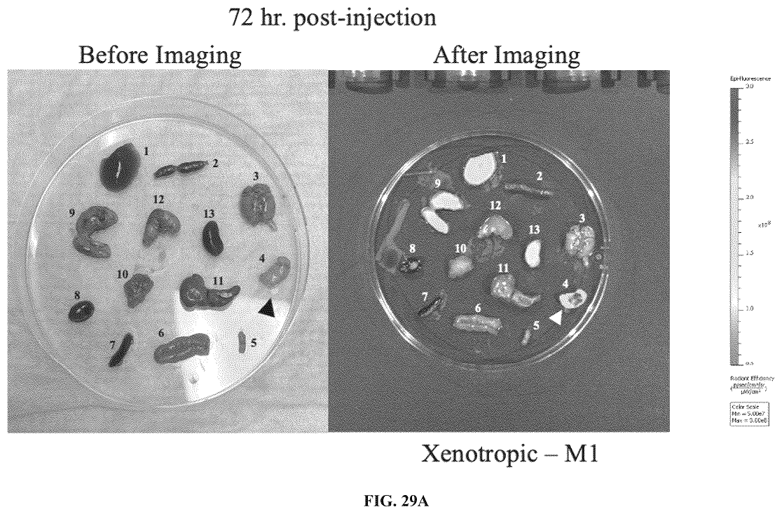

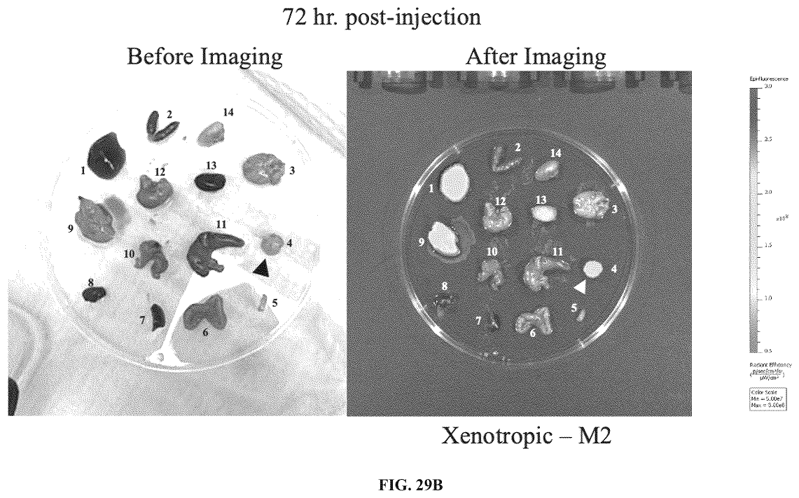

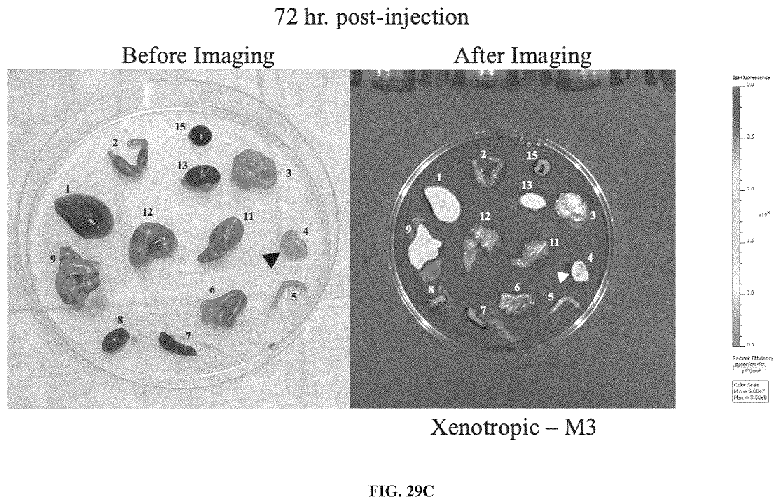

FIGS. 28A, B and C show distribution of CF-750 GalT-V antibody fluorescence in individual tissues from a subcutaneous/xenograft tumor bearing mice. Liver (1), colon (2), brain (3), tumor (4), sigmoid colon (5), large intestine (6), spleen (7), heart (8), lungs (9), small intestine (10), cecum (11), stomach (12), kidney (13), muscle (14), and blood (15).

At 72 hours, post injection of CF-750-GalT-V antibody, three xenotropic tumor bearing mice (M1, M2, M3) were euthanized and individual organs were excised and photographed (FIG. 28A, B, C). First, the individual organs were photographed (left panel FIG. 28A, B, C) and then xenotropic images were taken. Note that the liver, spleen, and kidney took up significant fluorescence. But the tumor tissue (shown by a white arrow) accumulated the largest amount of the fluorescence.

In sum, our in vivo studies reveal that fluorescence tagging of GalT-V antibody is a valuable reagent to non-invasively image CRC tumors in mice. Over time, the CF-750-GalT-V antibody is primarily concentrated in the tumor tissue. As the liver, kidney, and lungs are involved in the metabolism and subsequent excretion of the antibody, significant fluorescence was also observed in these tissues. This additional information may be useful in determining the therapeutic efficacy of the GalT-V antibody in mitigating kidney and liver cancer, in addition to CRC. Also, CF-750-GalT-V antibody could be used as a diagnostic tool in human CRC and other cancer tissues wherein GalT-V enrichment may occur.

Statistical Analysis: Statistical analysis of the data was performed using singular/multiple unpaired t-tests with the GraphPad Prism 9 Software.

### Conclusions

1. Immuno-histochemical studies show that in human HCT-116 cells GalT-V is co-localized on the cell-surface with caveolin.
2. As the incubation temperature is shifted from 4° C. to 37° C., the cell-surface bound GalT-V is internalized and resides in the cytoplasm. Weak immunostaining is associated with the Golgi apparatus due to low abundance.
3. Binding and internalization studies using [⁸⁹Zr] GalT-V antibody showed concentration-dependent and time-dependent binding to HCT-116 cells and HCAEC cells.
4. Binding was specific to the GalT-V antigen.
5. Binding was dependent on the cellular level of GalT-V as: a. HCT-116 cells significantly bound more of the GalT-V antibody compared to HCAEC. b. D-PDMP-treated HCT-116 cells bound less than untreated cells. Our previous study show that D-PDMP reduces GalT-V mass.

In human colorectal cells GalT-V is localized on the cell-surface allowing the biding of corresponding [⁸⁹Zr] GalT-V antibody and its subsequent internalization.

### Other Embodiments

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A pharmaceutical composition comprising a therapeutically effective amount of an antibody, wherein the antibody specifically binds to a β-1,4-galactosyltransferase-V (β-1,4-GalT-V) epitope, the antibody comprising:
a heavy chain variable region sequence having at least an 80% amino acid sequence identity to
(SEQ ID NO: 3)
EVQLEQSGAELARPGASVKLSCRTSGYTFTNYWMQWIKQRPGQGLEWIG
AMHPGRAYIRYNQKFQGKATLTADKSSSTAYMQLNSLASEDSAVYYCAR
WSDYDYWGQGTTLTVSS,
and/or
a light chain variable sequence having at least a 80% amino acid sequence identity to
(SEQ ID NO: 4)
DVVMTQTPPTLSVTIGQPASISCKSSQSLLDSDGKTYLNWLLQRPGQSP
KRLIYLVSKLGSGVPDRFTGSGSGTDFTLKISRVEAEDLGVYYCWQGTH
FPRTFGGGTKLEIKR.

2-3. (canceled)

4. The pharmaceutical composition of claim 1, wherein the antibody comprises a heavy chain variable region sequence having an amino acid sequence set forth in SEQ ID NO: 3.

5. The pharmaceutical composition of claim 1, wherein the antibody comprises a light chain variable region sequence having an amino acid sequence set forth in SEQ ID NO: 4.

6. The pharmaceutical composition of claim 1, further comprising one or more secondary therapeutic agents.

7-9. (canceled)

10. The pharmaceutical composition of claim 1, further comprising a peptide having at least a 80% sequence identity to
(SEQ ID NO: 5)
IGAQVYEQVLRSAYAKRNSSVND.

11. The pharmaceutical composition of claim 1, further comprising a peptide having at least a 90% sequence identity to
(SEQ ID NO: 5)
IGAQVYEQVLRSAYAKRNSSVND.

12. The pharmaceutical composition of claim 1, wherein the antibody is humanized.

13. A humanized antibody is provided that can that specifically bind to a β-1,4-galactosyltransferase-V (β-1,4-GalT-V) epitope.

14. A pharmaceutical composition comprising a humanized antibody of claim 13.

15. An expression vector comprising a heavy chain variable region sequence nucleic acid sequence having at least an 80% sequence identity to
(SEQ ID NO: 1)
gaagttcagctggagcagtctggggctgaactggctagacctggggctt
cagtgaagttgtcctgtaggacttctggctacacctttacaaactactg
gatgcagtggattaaacagaggcctggacagggtctggaatggattggg
gctatgcatcctggacgtgcgtatattaggtacaaccagaagttccagg
gcaaggccacattgactgcagataaatcctccagcacagcttacatgca
actcaacagcttggcatctgaggactctgcggtctattactgtgcaaga
tggagtgactacgactactggggtcaaggcaccactctcacagtctcct
ca;
or
An expression vector comprising a light chain variable region sequence nucleic acid sequence having at least an 80% sequence identity to
(SEQ ID NO: 2)
gatgttgtgatgacccagactccactcactttgtcggttaccattggac
aaccagcctccatctcttgcaagtcaagtcagagcctcttagatagtga
tggaaagacatatttgaattggttgttacagaggccaggccagtctcca
aagcgcctaatctatctggtgtctaaactgggctctggagtccctgaca
ggttcactggcagtggatcagggacagatttcacactgaaaatcagcag
agtggaggctgaggatttgggagtttattattgctggcaaggtacacat
tttcctcggacgttcggtggaggcaccaagctggaaatcaaacgg;
or
An expression vector comprising (i) a heavy chain variable region sequence nucleic acid sequence having at least an 80% sequence identity to SEQ ID NO: 3, and (ii) a light chain variable region sequence nucleic acid sequence having at least a 80% sequence identity to SEQ ID NO: 2.

16-27. (canceled)

28. A pharmaceutical composition comprising a therapeutically effective amount of a synthetic peptide comprising an amino acid sequence having at least a 90% amino acid sequence to SEQ ID NO: 5 and at least one adjuvant; or
A pharmaceutical composition comprising a therapeutically effective amount of:
(i) an antibody comprising (a) a heavy chain variable region sequence nucleic acid sequence having at least a 80% sequence identity to SEQ ID NO: 3, and/or (b) a light chain variable region sequence nucleic acid sequence having at least a 80% sequence identity to SEQ ID NO: 2;
(ii) a synthetic peptide comprising an amino acid sequence having at least a 80% amino acid sequence to SEQ ID NO: 5;
(iii) an adjuvant or pharmaceutically acceptable carrier, or
A pharmaceutical composition comprising a therapeutically effective amount of:
(a) an antibody, wherein the antibody specifically binds to a β-1,4-galactosyltransferase-V (β-1,4-GalT-V) epitope, the antibody comprising:
(i) a heavy chain variable region sequence having at least an 80% amino acid sequence identity to:
(SEQ ID NO: 3)
EVQLEQSGAELARPGASVKLSCRTSGYTFTNYWMQWIKQRPGQGLEWIG
AMHPGRAYIRYNQKFQGKATLTADKSSSTAYMQLNSLASEDSAVYYCA
RWSDYDYWGQGTTLTVSS,
and/or
(ii) a light chain variable sequence having at least an 80% amino acid sequence identity to:
(SEQ ID NO: 4)
DVVMTQTPPTLSVTIGQPASISCKSSQSLLDSDGKTYLNWLLQRPGQSP
KRLIYLVSKLGSGVPDRFTGSGSGTDFTLKISRVEAEDLGVYYCWQGTH
FPRTFGGGTKLEIKR,
and,
(b) a therapeutically effective amount of at least one inhibitor of glycosphingolipid synthesis.

29-39. (canceled)

40. A method of generating an immune response to β-1,4-galactosyltransferase-V (β-1,4-GalT-V) in a subject in need thereof, comprising administering a therapeutically effective amount of a synthetic peptide comprising an amino acid sequence having at least an 80% amino acid sequence to SEQ ID NO: 5; and an adjuvant.

41. (canceled)

42. A method of treating colorectal cancer comprising administering to a subject suffering from or susceptible to colorectal cancer an antibody that specifically binds to a β-1,4-galactosyltransferase-V (β-1,4-GalT-V) epitope.

43. The method of claim 42 wherein the administered antibody is a humanized antibody that specifically binds to a β-1,4-galactosyltransferase-V (β-1,4-GalT-V) epitope.

44. A method of treating colorectal cancer comprising administering to a subject suffering from or susceptible to colorectal cancer a pharmaceutical composition, antibody, expression vector or peptide of claim 1.

45. A method of treating colorectal cancer, comprising administering to a subject a pharmaceutical composition comprising an antibody comprising (a) a heavy chain variable region sequence nucleic acid sequence having at least an 80% sequence identity to SEQ ID NO: 3, and/or (b) a light chain variable region sequence nucleic acid sequence having at least an 80% sequence identity to SEQ ID NO: 2.

46-55. (canceled)

56. A method of treating diabetes, atherosclerosis, obesity, autoimmune diseases, or diseases associated with abnormal levels of β-1,4-galactosyltransferase-V (β-1,4-GalT-V), comprising administering to a subject in need thereof, a pharmaceutical composition, antibody, expression vector or peptide of claim 1, or combinations thereof.

57. A method of diagnosing and treating colorectal cancer, comprising:
measuring levels of β-1,4-galactosyltransferase-V (β-1,4-GalT-V) and/or glycosphingolipids in a subject's biological sample, wherein increase β-1,4-GalT-V and/or GSL levels as compared to a healthy subject are elevated, wherein elevated β-1,4-GalT-V and/or GSL levels are diagnostic of colorectal cancer;
administering to the subject diagnosed with colorectal cancer, a pharmaceutical composition, antibody, expression vector or peptide of claim 1, or combinations thereof, thereby,
treating colorectal cancer.

58-59. (canceled)

60. A method of monitoring the progression and treatment of colorectal cancer in a subject, comprising:
administering to the subject diagnosed with colorectal cancer, a pharmaceutical composition, antibody, expression vector or peptide of claim 1, or combinations thereof,
measuring levels of β-1,4-galactosyltransferase-V (β-1,4-GalT-V) and/or glycosphingolipids in a subject's biological sample, wherein a decrease in β-1,4-GalT-V and/or GSL levels as compared to a baseline, are indicative of a decrease in colorectal cancer cells and treatment of the colorectal cancer,
thereby monitoring the progression and treatment of colorectal cancer;

61. (canceled)

62. A method of treating a subject suffering from or susceptible to macular degeneration, comprising administering to the subject an effective amount of a pharmaceutical composition, antibody, expression vector or peptide of claim 1, or combinations thereof.

63. (canceled)

64. A method of treating a subject suffering from or susceptible to Alzheimer' disease, comprising administering to the subject an effective amount of a pharmaceutical composition, antibody, expression vector or peptide of claim 1, or combinations thereof.

65. (canceled)

66. A method of treating a subject suffering from or susceptible to migraines or migraine pain, comprising administering to the subject an effective amount of a pharmaceutical composition, antibody, expression vector or peptide of claim 1, or combinations thereof.

67. (canceled)

68. A method of treating a subject suffering from or susceptible to Metabolic syndrome, comprising administering to the subject an effective amount of a pharmaceutical composition, antibody, expression vector or peptide of claim 1, or combinations thereof.

69-84. (canceled)


SEQUENCE LISTING (9 sequences)

SEQ ID NO: 1 - DNA, 345 bp, synthetic construct
  source 1..345: mol_type = other DNA
gaagttcagctggagcagtctggggctgaactggctagacctggggcttcagtgaagttgtcctgtaggacttctggctacacctttacaaactactggatgcagtggattaaacagaggcctggacagggtctggaatggattggggctatgcatcctggacgtgcgtatattaggtacaaccagaagttccagggcaaggccacattgactgcagataaatcctccagcacagcttacatgcaactcaacagcttggcatctgaggactctgcggtctattactgtgcaagatggagtgactacgactactggggtcaaggcaccactctcacagtctcctca

SEQ ID NO: 2 - DNA, 339 bp, synthetic construct
  source 1..339: mol_type = other DNA
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactgggctctggagtccctgacaggttcactggcagtggatcagggacagatttcacactgaaaatcagcagagtggaggctgaggatttgggagtttattattgctggcaaggtacacattttcctcggacgttcggtggaggcaccaagctggaaatcaaacgg

SEQ ID NO: 3 - protein, 115 aa, synthetic construct
EVQLEQSGAELARPGASVKLSCRTSGYTFTNYWMQWIKQRPGQGLEWIGAMHPGRAYIRYNQKFQGKATLTADKSSSTAYMQLNSLASEDSAVYYCARWSDYDYWGQGTTLTVSS

SEQ ID NO: 4 - protein, 113 aa, synthetic construct
DVVMTQTPPTLSVTIGQPASISCKSSQSLLDSDGKTYLNWLLQRPGQSPKRLIYLVSKLGSGVPDRFTGSGSGTDFTLKISRVEAEDLGVYYCWQGTHFPRTFGGGTKLEIKR

SEQ ID NO: 5 - protein, 23 aa, synthetic construct
IGAQVYEQVLRSAYAKRNSSVND

SEQ ID NO: 6 - DNA, 345 bp, synthetic construct; identical to SEQ ID NO: 1
  source 1..345: mol_type = other DNA

SEQ ID NO: 7 - protein, 115 aa, synthetic construct; identical to SEQ ID NO: 3

SEQ ID NO: 8 - DNA, 339 bp, synthetic construct; identical to SEQ ID NO: 2
  source 1..339: mol_type = other DNA

SEQ ID NO: 9 - protein, 113 aa, synthetic construct
DVVMTQTPLTLSVTIGQPASISCKSSQSLLDSDGKTYLNWLLQRPGQSPKRLIYLVSKLGSGVPDRFTGSGSGTDFTLKISRVEAEDLGVYYCWQGTHFPRTFGGGTKLEIKR